(19) United States
(12) Patent Application Publication
(10) Pub. No.: US 2026/0240810 A1
(43) Pub. Date: Aug. 20, 2026

(54) COMPOSITIONS AND METHODS FOR REDUCING ADVERSE SIDE EFFECTS IN CANCER TREATMENT

(71) Applicant: EpicentRx, Inc., La Jolla, CA (US)

(72) Inventors: Bryan ORONSKY, Los Altos Hills, CA (US); Scott CAROEN, San Francisco, CA (US); Tony R. REID, San Diego, CA (US)

(21) Appl. No.: 18/874,433

(22) PCT Filed: Jun. 12, 2023
(86) PCT No.: PCT/US2023/068295

§ 371 Date: Dec. 12, 2024

Related U.S. Application Data
(60) Provisional application No. 63/351,769, filed on Jun. 13, 2022.

(51) Int. Cl. A61K 31/397; A61K 31/167; A61K 31/19; A61K 31/195; A61K 31/337; A61K 31/355; A61K 31/4045; A61K 31/416; A61K 31/4164; A61K 31/4178; A61K 31/4704; A61K 31/4745; A61K 31/506; A61K 31/513; A61K 31/522; A61K 31/555; A61K 31/573; A61K 31/66; A61K 31/7024; A61K 31/704; A61K 35/18; A61K 45/06; A61P 39/00

(52) U.S. Cl. CPC A61K 31/397; A61K 31/167; A61K 31/19; A61K 31/195; A61K 31/337; A61K 31/355; A61K 31/4045; A61K 31/416; A61K 31/4164; A61K 31/4178; A61K 31/4704; A61K 31/4745; A61K 31/506; A61K 31/513; A61K 31/522; A61K 31/555; A61K 31/573; A61K 31/66; A61K 31/7024; A61K 31/704; A61K 35/18; A61K 45/06; A61P 39/00

(57) ABSTRACT

Compositions and methods for reducing adverse side effects in cancer treatment are provided. A method of treating and preventing injury from ionizing radiation, chemotherapy or a combination of radiation and chemotherapy includes administering a prophylactically or therapeutically effective amount of RRx-001 that also enhances cytotoxicity to tumors.

### CROSS-REFERENCE TO RELATED APPLICATIONS SECTION

This application is a U.S. Non-Provisional Patent Application that claims priority to U.S. Provisional Patent Application Ser. No. 63/351,769 filed on Jun. 13, 2022, the entire contents of which are hereby incorporated by reference in their entirety.

### FIELD OF THE INVENTION

This disclosure relates to methods for treating and preventing radiation and/or chemotherapy related injury and/or afflictions, such as mucositis, oral dysphagia, esophagitis, and gastrointestinal distress, by administering a prophylactically or therapeutically effective amount of RRx-001. The present disclosure also relates to methods for improving the therapeutic efficacy of therapeutic agents previously limited by suboptimal therapeutic performance or poor tolerability by either improving efficacy as monotherapy or reducing side effects.

### BACKGROUND

The treatment of cancer via chemotherapy and/or radiation is often complicated by the development of various local and systemic adverse side effects, such as oral mucositis (or OM), dysphagia, dyspepsia, laryngeal inflammation, oral dysesthesia, vomiting, salivary duct inflammation, esophagitis, any gastrointestinal distress, dermatitis, hair loss or increased creatinine. Oral mucositis, in particular, is a painful and debilitating dose limiting toxicity of chemotherapy and radiation treatment, characterized by ulcerative lesions in the oral mucosa, and for which no standard intervention or preventative measure currently exists. When administered with concomitant chemotherapy (CRT), the incidence of severe forms of oral mucositis approaches 70% and lasts for about three weeks. See M. Bonomi, et al. (2023) Int. J. Radiat. Oncol. Biol. Phys. 116(3):551-559. Further, close to 100% of head and neck cancer patients (HNC) treated with both chemo- and radiotherapy develop at least moderate mucositis, and more than two-thirds suffer from severe forms of it. Severe mucositis is associated with hospitalization and treatment delay or discontinuation as well as nutritional compromise from pain, discomfort and, in some cases, an inability to swallow along with the need for gastrostomy feedings, bacteremia and sepsis risk, a reduction of the quality of life, a worse prognosis and an increase in patient management costs. See B. Oronsky, et al. (2018) Transl Oncol., 11(3):771-778.

Two events are critical to the initiation of radiation-induced injury: oxidative stress and activation of the innate immune response. Both events lead to the release of damaging tissue mediators including proinflammatory cytokines and matrix metalloproteinases. Given their importance as initiating mechanisms for CRT-induced oral mucositis, attenuation of oxidative stress or innate immune response activation are attractive druggable targets to prevent or mitigate oral mucositis development or progression. See M. Bonomi, et al. (2023) Int. J. Radiat. Oncol. Biol. Phys. 116(3):551-559.

All adverse side effects of cancer treatment are not only debilitating in the short term for a patient, leading to deterioration of quality of life, but can be directly life threatening or indirectly life threatening by causing a patient to stop or delay the treatment. In addition, the aftereffects of chemotherapy and radiation may include development of a serious chronic disease especially for survivors of childhood cancers. There is a need in the art for a better cancer treatment that reduces both acute and delayed adverse side effects and the subsequent development of acute or chronic disease or dysfunction. For chemotherapy, abundant evidence links the therapeutic effects to the amount of drug administered per unit time. “Dose intensity (DI)” represents the amount (mg/m²) of a drug administered per unit time (week) and measures the intensity of chemotherapy, which increases or decreases depending on the dose administered, the time interval of administration, or both. See G. H. Lyman (2009) J Natl Compr Canc Netw. 7(1):99-108. An indicator called “relative dose intensity (RDI)”, the ratio of the delivered dose intensity (dose per unit body surface area per unit time [mg/m² per week]) to the standard or planned dose intensity for a chemotherapy regimen, reflects whether the DI of a therapy was implemented as planned and is now commonly included in reports of clinical studies. See C. M. Nielson, et al. (2021) Oncologist. 26(9):e1609-e1618.

Further, there is a need for a treatment that mitigates adverse side effects and sequelae in various cancer treatments such as chemotherapy and radiation treatment. The use of current drugs or other approaches to counteract chemotherapy and radiation-induced adverse effects entails a difficult trade-off between the benefits they provide, however small, and the near-certainty of inducing other side-effects which only add to patient discomfort and intolerability. See K. Nurgali, et al. (2018) Front Pharmacol. 9:245. Moreover, and perhaps more importantly, the use of such drugs e.g., palifermin e.g., amifostine or similar approaches, raises questions about whether they increase or decrease the efficacy of anticancer agent(s) and whether protection of normal tissue and amelioration of toxicity also protects cancer cells. New strategies to improve tolerance and reduce sequelae of cancer chemotherapy and radiotherapy are urgently needed that do not simultaneously interfere with or reduce antineoplastic efficacy, especially in head and neck cancers.

These cancers, which typically begin in the squamous cells that line the mucosal surfaces of the oral cavity, nasal cavity, pharynx, and larynx, are often referred to as head and neck squamous cell carcinoma (HNSCC), representing the fifth most common cancer type and cause of cancer-related death worldwide. See L. Ries, et al. SEER Cancer Statistics Review, 1975-2005. 2008, National Cancer Institute: Bethesda, MD.

The majority of HNSCC cases (more than 90%) are related to exposure to carcinogens, including tobacco and alcohol (see W. J. Blot, et al. (1988) Cancer Res. 48(11):3282-7.), betel nut, Epstein-Barr Virus (EBV), and sexually transmitted viral pathogens such as human papillomavirus (HPV) (see U. Hording, et al. (1992) Int J Gynecol Cancer. 2(6):314-7.).

In addition to these risk factors, the incidence of HNSCC is significantly higher in individuals with inherited genetic syndromes such as Fanconi's Anemia, Xeroderma pigmentosum, Ataxia telangiectasia, Li Fraumeni Syndrome, Retinoblastoma, Dyskeratosis congenita, Bloom's Syndrome, and Rothmund-Thompson, etc. Of these, HNSCC (as well as other cancers of the skin, gastrointestinal system, genital tract, and acute myeloid leukemia) is significantly more common in Fanconi's Anemia (FA). See B. P. Alter (2003) Cancer. 97(2):425-40. Thus, there exists a need to prevent or delay the cancer susceptibility in FA (and other genetic syndromes) as well as to mitigate the DNA damage from chemoradiotherapy treatments, which increase the cancer risk considerably.

### SUMMARY

Compositions and methods for preventing or reducing adverse side effects in cancer treatment are provided. An exemplary embodiment is a method of treating or preventing normal tissue injury from ionizing radiation, chemotherapy or a combination of radiation and chemotherapy in a subject in need thereof by administering a prophylactically or therapeutically effective amount of RRx-001, or a pharmaceutically acceptable salt thereof, that also enhances cytotoxicity to tumors. In some embodiments, the subject is a mammal subject. The mammal subject may be a human subject or an animal subject.

In an embodiment, the therapeutically effective amount of the RRx-001, or a pharmaceutically acceptable salt thereof, is in a range of about 0.1 mg to about 500 mg. In another embodiment, the therapeutically effective amount of the RRx-001, or a pharmaceutically acceptable salt thereof, is in a range of about 0.5 mg to about 200 mg. In another embodiment, the therapeutically effective amount of the RRx-001, or a pharmaceutically acceptable salt thereof, is in a range of about 5 mg to about 50 mg. In another embodiment, the therapeutically effective amount of the RRx-001, or a pharmaceutically acceptable salt thereof, is in a range of about 10 mg to about 30 mg.

The prophylactically or therapeutically effective amount of the RRx-001, or a pharmaceutically acceptable salt thereof, may be provided as separate medicaments for administration at the same time or at different times. In some embodiments, the effective amount of RRx-001, or a pharmaceutically acceptable salt thereof, occurs via oral administration, transdermal administration, administration by inhalation, nasal administration, topical administration, intra-aural administration, rectal administration, intravenous administration, intramuscular administration, subcutaneous administration, or intraperitoneal administration, or combinations thereof. The prophylactically or therapeutically effective amount may be administered by intravenous injection. The prophylactically or therapeutically effective amount of RRx-001 may be mixed ex vivo with blood and administered by intravenous injection. The prophylactically or therapeutically effective amount may be administered by oral administration or swish and spit or swish and swallow. The prophylactically or therapeutically effective amount may be administered by direct intratumoral injection.

A patient or subject for treatment may have a locally advanced solid tumor including a gastrointestinal malignancy, head and neck cancer, gynecological cancer, breast cancer, hepatocellular cancer, esophageal cancer, lung cancer, genitourinary cancer, gastrointestinal tract cancers, genitourinary cancers, hepatocellular carcinoma, glioblastoma, or sarcoma. The normal tissue injury may include a condition selected from the group consisting of: mucositis, dysphagia, dyspepsia, laryngeal inflammation, oral dysesthesia, vomiting, salivary duct inflammation, esophagitis, any gastrointestinal distress, myelosuppression, impotence, infertility, dermatitis, hair loss, and increased creatinine. The normal tissue injury may include secondary cancer development. Normal tissue in the human body may be selectively protected relative to tumor tissue in the human body subsequent to the administering of the prophylactically or therapeutically effective amount of the RRx-001, or a pharmaceutically acceptable salt thereof. In some embodiments, the subject has a genetic syndrome that predisposes the subject to head and neck cancer, such as Fanconi's anemia, Xeroderma pigmentosum, Ataxia telangiectasia, Li Fraumeni Syndrome, Retinoblastoma, Dyskeratosis congenita, Bloom's Syndrome, or Rothmund-Thompson. In other embodiments, the subject has a head and neck cancer attributable to one or more of cigarette smoking, alcohol drinking, infection with HPV, or EBV.

In some embodiments, administering the therapeutically effective amount of the RRx-001, or a pharmaceutically acceptable salt thereof, is performed via a single administration. In other embodiments, administering the therapeutically effective amount of the RRx-001, or a pharmaceutically acceptable salt thereof, is performed via at least two administrations during a time period. In some embodiments, the time period is up to six weeks. In some embodiments, administering the therapeutically effective amount of the RRx-001, or a pharmaceutically acceptable salt thereof, is performed at a frequency of between about 1 time per hour and about 1 time per month during the time period. In other embodiments, administering the therapeutically effective amount of the RRx-001, or a pharmaceutically acceptable salt thereof, is performed at a frequency of between about 4 times per day and about 1 time per week during the time period. In some embodiments, administering the therapeutically effective amount of the RRx-001, or a pharmaceutically acceptable salt thereof, is performed at a frequency of between about 2 times per day and about 3 times per week during the time period. In some embodiments, administering the therapeutically effective amount of the RRx-001, or a pharmaceutically acceptable salt thereof, is performed at a frequency of about one time a day during the time period. In some embodiments, administering the therapeutically effective amount of the RRx-001, or a pharmaceutically acceptable salt thereof, is performed at a frequency of between about 1 time per day and about 2 times per week during the time period.

Administering a prophylactically or therapeutically effective amount of the RRx-001, or a pharmaceutically acceptable salt thereof, may include a pretreatment dose that is administered at a frequency during a time period prior to another treatment, such as ionizing radiation or chemotherapy. In some embodiments, the other treatment comprises the chemotherapy and/or immunotherapy, and wherein the chemotherapy and/or the immunotherapy comprises an agent selected from the group consisting of: Dexamethasone, Erbitux (Cetuximab), Hydroxyurea, Pembrolizumab, Nivolumab, Hydrocortisone, Prednisone, Cyclophosphamide, Methotrexate, Paclitaxel, Carboplatin, Etoposide, Gemcitabine, Cisplatin, Oxaliplatin, Chlorambucil, Mechloethamine, Melphalan, Tocilizumab, Brentuximab Vedotin, Doxorubicin, Afatinib, Everolimus, Netupitant, Palonosetron, Imiquimod, and Fluorouracil. In some embodiments, an amount of the agent is in a range of about 1 mg to about 50 mg. In other embodiments, the amount of the agent is in the range of about 1 mg to about 15 mg. The pretreatment dose may be administered over a period of between about 1 day and about 6 months or between about 1 week and 4 weeks. In some embodiments, the pretreatment dose may be administered over a time period of about 2 weeks. The pretreatment dose of the RRx-001, or a pharmaceutically acceptable salt thereof, may include between about 1 mg and about 200 mg. The pretreatment dose of the RRx-001, or a pharmaceutically acceptable salt thereof, may include between about 2 mg and about 20 mg. The pretreatment dose may be administered a frequency of between about 1 time per week, 1 time per month, 2-5 times per week, 2-4 times per month and more than once per day. In various embodiments, no RRx-001, or a pharmaceutically acceptable salt thereof, may be administered subsequent to the ionizing radiation or chemotherapy. In some embodiments, the method further comprises administering at least one additional dose of the RRx-001, or a pharmaceutically acceptable salt thereof, concurrently with the other treatment. In some embodiments, the method further comprises administering at least one additional dose of the RRx-001, or a pharmaceutically acceptable salt thereof, subsequent the other treatment.

In some embodiments, the method comprises administering the therapeutically effective amount of the RRx-001, or a pharmaceutically acceptable salt thereof, as a pretreatment dose of the RRx-001, or a pharmaceutically acceptable salt thereof. In some embodiments, the amount of the pretreatment dose of the RRx-001, or a pharmaceutically acceptable salt thereof, is in a range of about 1 mg to about 200 mg. In some embodiments, the amount of the pretreatment dose of the RRx-001, or a pharmaceutically acceptable salt thereof, is in a range of about 2 mg to about 20 mg. In this embodiment, the therapeutically effective amount of the RRx-001, or a pharmaceutically acceptable salt thereof, is not administered during the treatment period in which the subject is treated with the chemotherapy and/or the IMRT (e.g., it is used only as a pretreatment and not as a cotreatment).

In some embodiments, the method further comprises administering an agent prior to, concurrently with, or subsequent administering the effective amount of RRx-001, or a pharmaceutically acceptable salt thereof. In some embodiments, administering the agent occurs during a time period in a range of about 1 week to about 6 weeks prior to administering the effective amount of RRx-001, or a pharmaceutically acceptable salt thereof. In some embodiments, the agent comprises a corticosteroid, such as dexamethasone. In some embodiments, an amount of the agent is in a range of about 0.1 mg to about 50 mg. In some embodiments, the agent is selected from the group consisting of: a PARP inhibitor, a tyrosine kinase inhibitor, a thiol-based chemoradioprotectant agent, an EGFR inhibitor, a histone deacetylase (HDAC) inhibitor, a DNA methyltransferase inhibitor, fluorouracil (5-FU), imatinib, hydroxyurea, taxol, an oncolytic virus, a checkpoint inhibitor, and a topoisomerase inhibitor. In some embodiments, the agent comprises the thiol-based chemoradioprotectant agent, and wherein the thiol-based chemoradioprotectant agent is selected from the group consisting of: N-acetyl cysteine (NAC), amifostine, sodium thiosulfate (STS), D-methionine, GSH ethyl ester, and GlyNAC (Glycine and N-Acetylcysteine). In some embodiments, the agent comprises the topoisomerase inhibitor, and wherein the topoisomerase inhibitor is selected from the group consisting of: irinotecan and doxorubicin. In some embodiments, the agent treats oral mucositis and is selected from the group consisting of: GM-CSF, palifermin, pilocarpine, gabapentin, keratinocyte growth factor (KGF), coconut oil, MucoLox, cetuximab, a checkpoint inhibitor, transforming growth factor beta (TGF-β), an epidermal growth factor, honey, benzydamine, a corticosteroid, rebamipide, an antimicrobial agent, misoprostol, Amifostine, brilacidin, L-glutamine, low-level laser therapy, laser and light therapy, cryotherapy, vitamin E, pentoxyfyilline, GC4419, clonidine, melatonin, a probiotic, actovegin, aloe vera, allopurinol, azithromycin, Black Mulberry Molasses, Glycerin payayor, Qingre Liyanmo decoction, erythropoietin mouthwash, EC-18, sucralfate, Avasopasem manganese, and lidocaine. In some embodiments, the checkpoint inhibitor is selected from the group consisting of: pembrolizumab and nivolumab. In some embodiments, the antimicrobial agent is selected from the group consisting of: chlorhexidine, polymyxin E, and amphotericin.

Administering the prophylactically or therapeutically effective amount of the RRx-001, or a pharmaceutically acceptable salt thereof, may include concurrent administration of RRx-001 with the ionizing radiation, chemotherapy or a combination of radiation and chemotherapy. An amount of the RRx-001, or a pharmaceutically acceptable salt thereof, that is administered concurrently with the ionizing radiation, chemotherapy or a combination of radiation and chemotherapy may be between about 1 mg and about 200 mg. An amount of the RRx-001, or a pharmaceutically acceptable salt thereof, that is administered concurrently with the ionizing radiation, chemotherapy or a combination of radiation and chemotherapy may be about 4 mg. The therapeutically effective amount of the RRx-001, or a pharmaceutically acceptable salt thereof, that is administered concurrently with the ionizing radiation, chemotherapy or a combination of radiation and chemotherapy may be administered a frequency of between about 1 time per week, 1 time per month, 2-5 times per week, 2-4 times per month and more than once per day.

In some embodiments, administering the effective amount of the RRx-001, or a pharmaceutically acceptable salt thereof, comprises administering a pretreatment dose of the RRx-001, or a pharmaceutically acceptable salt thereof, at a frequency during a time period prior to another treatment. In some embodiments, the other treatment comprises at least one of radiation and chemotherapy. In some embodiments, the other treatment comprises the radiation, and the radiation comprises ionizing radiation. In some embodiments, the other treatment comprises the chemotherapy and/or immunotherapy, and wherein the chemotherapy and/or the immunotherapy comprises an agent selected from the group consisting of: Dexamethasone, Erbitux (Cetuximab), Hydroxyurea, Pembrolizumab, Nivolumab, Hydrocortisone, Prednisone, Cyclophosphamide, Methotrexate, Paclitaxel, Carboplatin, Etoposide, Gemcitabine, Cisplatin, Oxaliplatin, Chlorambucil, Mechloethamine, Melphalan, Tocilizumab, Brentuximab Vedotin, Doxorubicin, Afatinib, Everolimus, Netupitant, Palonosetron, Imiquimod, and Fluorouracil. In some embodiments, an amount of the agent is in a range of about 1 mg to about 50 mg. In some embodiments, the amount of the agent is in the range of about 1 mg to about 15 mg. In some embodiments, the time period is in a range of about 1 day to about 6 months. In some embodiments, the time period is in a range of about 1 week to about 4 weeks. In some embodiments, the time period is about 2 weeks. In some embodiments, an amount of the pretreatment dose of the RRx-001, or a pharmaceutically acceptable salt thereof, is in a range of about 1 mg to about 200 mg. In some embodiments, the amount of the pretreatment dose of the RRx-001, or a pharmaceutically acceptable salt thereof, is in a range of about 2 mg to about 20 mg. In some embodiments, the frequency is about 1 time per week, 1 time per month, 2-5 times per week, 2-4 times per month, one time per day, two times per day, three times per day, or four times per day.

Another general aspect is a method of increasing tumor ablation in a subject undergoing treatment with chemotherapy and/or radiotherapy. The method includes contacting tissue of the subject with a prophylactically or therapeutically effective amount of the RRx-001, or a pharmaceutically acceptable salt thereof. The contacting may include providing the prophylactically or therapeutically effective amount as separate medicaments at the same time or at different times. The contacting may include administering the prophylactically or therapeutically effective amount by intravenous injection. The prophylactically or therapeutically effective amount of the RRx-001, or a pharmaceutically acceptable salt thereof, may be mixed ex vivo with blood and administered by intravenous injection. The prophylactically or therapeutically effective amount may be administered by oral administration or swish and spit or swish and swallow. The prophylactically or therapeutically effective amount may be administered by direct tumor injection. The subject may have a locally advanced solid tumor including a gastrointestinal malignancy, head and neck cancer, gynecological cancer, breast cancer, hepatocellular cancer, esophageal malignancy, hepatocellular carcinoma, genitourinary cancer, lung cancer, genitourinary cancer, glioblastoma, or sarcoma. Secondary cancer development in the subject may be attenuated subsequent to contacting tissue with the RRx-001, or a pharmaceutically acceptable salt thereof. Normal tissue in the subject may be selectively protected relative to tumor tissue in the subject subsequent to the contacting tissue with the prophylactically or therapeutically effective amount of the RRx-001, or a pharmaceutically acceptable salt thereof. Contacting tissue of the subject may include a pretreatment dose of the RRx-001, or a pharmaceutically acceptable salt thereof, that is administered prior to ionizing radiation or chemotherapy. The pretreatment dose may be administered over a period of between about 1 day and about 6 months. The pretreatment does not be between about 1 mg and about 200 mg. The pretreatment dose may be between about 2 mg and about 10 mg. In various embodiments, the RRx-001, or a pharmaceutically acceptable salt thereof, is not administered to the subject subsequent to a start of treatment with chemotherapy and/or radiotherapy. Contacting tissue of the subject may include a concurrent dose of the RRx-001, or a pharmaceutically acceptable salt thereof, that is administered during treatment with chemotherapy and/or radiotherapy. The concurrent dose may be between about 1 mg and about 200 mg. The concurrent dose may be about 4 mg. The concurrent dose may be administered a frequency of between about 1 time per week, 1 time per month, 2-5 times per week, 2-4 times per month and more than once per day.

An exemplary embodiment is a method for maximizing or increasing a tolerated dose of chemoradiotherapy in a treatment of a human body for cancer. The method includes administering an effective therapeutic amount of the RRx-001, or a pharmaceutically acceptable salt thereof, in combination with or prior to cisplatin and radiotherapy. Maximizing or increasing a tolerated dose may include maximizing or increasing a previously poorly tolerated dose. A maximum or increased tolerated amount or relative dose intensity (RDI) of at least one of cisplatin and radiotherapy may increase subsequent to the administering of the effective therapeutic amount of the RRx-001, or a pharmaceutically acceptable salt thereof. A previously poorly tolerated amount of at least one of cisplatin and radiotherapy may become tolerable subsequent to the administering of the effective therapeutic amount of the RRx-001, or a pharmaceutically acceptable salt thereof. A maximum or increased tolerated amount may include administering an increased dose of at least one of cisplatin and radiotherapy each time the at least one of cisplatin and radiotherapy are administered. The maximum or increased tolerated amount of cisplatin each time the cisplatin is administered may be between about 50 mg and about 150 mg.

Another general aspect is a method for increasing an anti-tumor efficacy of cisplatin and radiotherapy in a treatment for cancer. The method includes administering an effective therapeutic amount of the RRx-001, or a pharmaceutically acceptable salt thereof, in combination with an effective therapeutic amount of cisplatin and radiotherapy. The effective therapeutic amount may be provided as separate medicaments for administration at the same time or at different times. The effective therapeutic amounts of the RRx-001, or a pharmaceutically acceptable salt thereof, and chemoradiotherapy may be administered by intravenous injection. The effective therapeutic amount of the RRx-001, or a pharmaceutically acceptable salt thereof, may be administered by intratumoral injection. The effective therapeutic amount of the RRx-001, or a pharmaceutically acceptable salt thereof, may be mixed ex vivo with blood and administered by intravenous injection. The therapeutically effective amount of the RRx-001, or a pharmaceutically acceptable salt thereof, may be administered by oral administration or swish and spit or swish and swallow. A patient for the treatment may have a locally advanced solid tumor including a gastrointestinal malignancy, head and neck cancer, gynecological cancer, lung cancer, breast cancer, hepatocellular cancer, esophageal cancer, genitourinary cancer, glioblastoma, or sarcoma. Adverse side effects of a cancer therapeutic may be reduced subsequent to the administering of the therapeutically effective amount of RRx-001. The adverse side effects may be mucositis, dysphagia, dyspepsia, laryngeal inflammation, oral dysesthesia, vomiting, salivary duct inflammation, esophagitis, any gastrointestinal distress, myelosuppression, impotence, infertility, dermatitis, hair loss or increased creatinine. Secondary cancer development may be attenuated subsequent to the administering of the RRx-001. Normal tissue in the human body may be selectively protected relative to tumor tissue in the human body subsequent to the administering of the RRx-001, or a pharmaceutically acceptable salt thereof. The pretreatment dose may be administered over a period of between about 1 day and about 6 months. The dose of the RRx-001, or a pharmaceutically acceptable salt thereof, that is administered prior to cisplatin and radiotherapy may be between about 1 mg and about 200 mg (e.g., per day). The dose of the RRx-001, or a pharmaceutically acceptable salt thereof, that is administered prior to cisplatin and radiotherapy may be between about 2 mg and about 6 mg (e.g., per day). The dose of the RRx-001, or a pharmaceutically acceptable salt thereof, that is administered prior to cisplatin and radiotherapy may be about 4 mg (e.g., per day). The therapeutically effective amount of the RRx-001, or a pharmaceutically acceptable salt thereof, that is administered prior to cisplatin and radiotherapy may be administered a frequency of between about 1 time per week, 1 time per month, 2-5 times per week, 2-4 times per month and more than once per day. The dose of the RRx-001, or a pharmaceutically acceptable salt thereof, that is administered in combination with cisplatin and radiotherapy may be between about 1 mg and about 200 mg (e.g., per day). The dose of the RRx-001, or a pharmaceutically acceptable salt thereof, that is administered in combination with cisplatin and radiotherapy may be about 4 mg (e.g., per day). The therapeutically effective amount of the RRx-001, or a pharmaceutically acceptable salt thereof, that is administered in combination with cisplatin and radiotherapy may be administered a frequency of between about 1 time per week, 1 time per month, 2-5 times per week, 2-4 times per month and more than once per day.

In some embodiments, the therapeutically effective amount of the RRx-001, or a pharmaceutically acceptable salt thereof, is administered as a composition comprising a blood product. In some embodiments, the blood product comprises erythrocyte cells. In some embodiments, the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis. In some embodiments, the blood product is a mixture of packed red blood cells. In some embodiments, the blood product is whole blood. In some embodiments, the whole blood is autologous whole blood or donor-matched allogenic whole blood.

An exemplary embodiment is a composition to improve the efficacy and/or reduce the side effects of drug therapy. The composition includes a therapeutically effective quantity of the RRx-001, or a pharmaceutically acceptable salt thereof, the blood product, and an additional therapeutic agent. The additional therapeutic agent may be selected from the group consisting of PARP inhibitors, a tyrosine kinase inhibitor, an EGFR inhibitor, an HDAC inhibitor, a DNA methyltransferase inhibitor, 5-FU, imatinib, hydroxyurea, taxol, an oncolytic virus, a checkpoint inhibitor, and a topoisomerase inhibitor such as irinotecan or doxorubicin. RRx-001 may be combined with a thiol-based chemoradioprotectant agent. The thiol-based chemoradioprotectant agent may be selected from N-acetyl cysteine (NAC), amifostine, sodium thiosulfate (STS), D-methionine, GSH ethyl ester, and GlyNAC (Glycine and N-Acetylcysteine). Administration of the RRx-001, or a pharmaceutically acceptable salt thereof (e.g., a therapeutically effective amount), prior to or concurrent with the thiol-based chemoradioprotectant may prevent or reduce the side effects from the thiol based chemoradioprotectant. The therapeutically effective amount of the RRx-001, or a pharmaceutically acceptable salt thereof, that is administered prior to or concurrent with other agents may be used to treat oral mucositis. At least one of the other agents used to treat oral mucositis may be selected from the group consisting of: GM-CSF, palifermin, pilocarpine, gabapentin, keratinocyte growth factor (KGF), coconut oil, MucoLox, cetuximab, a checkpoint inhibitor, transforming growth factor beta (TGF-β), an epidermal growth factor, honey, benzydamine, a corticosteroid, rebamipide, an antimicrobial agent, misoprostol, Amifostine, brilacidin, L-glutamine, low-level laser therapy, laser and light therapy, cryotherapy, vitamin E, pentoxyfyilline, GC4419, clonidine, melatonin, a probiotic, actovegin, aloe vera, allopurinol, azithromycin, Black Mulberry Molasses, Glycerin payayor, Qingre Liyan decoction, erythropoietin mouthwash, EC-18, sucralfate, Avasopasem manganese, and lidocaine. In some embodiments, the checkpoint inhibitor is selected from the group consisting of: pembrolizumab and nivolumab. In some embodiments, the antimicrobial agent is selected from the group consisting of: chlorhexidine, polymyxin E, and amphotericin.

A patient or animal subject may have a lower incidence of malnutrition subsequent to administration of RRx-001. A patient or animal subject may have a lower incidence of gastrostomy-tube placement subsequent to administration of the therapeutically effective amount of the RRx-001, or a pharmaceutically acceptable salt thereof. A patient or animal subject may have a lower incidence of weight loss subsequent to administration of the therapeutically effective amount of the RRx-001, or a pharmaceutically acceptable salt thereof.

Toxicities associated with cancer therapy typically occur in clusters. It is rare for patients to develop only one side effect. In the case of concomitant chemoradiation for head and neck cancers, in addition to mucositis, patients are prone to damage to the parotid glands and ducts and radiation-induced dermatitis with consequent xerostomia, dysphagia, weight loss, and increased risk of candidiasis. The pathobiological basis for these changes is shared with those for mucositis. As such, in some embodiments, administration of the therapeutically effective amount of the RRx-001, or a pharmaceutically acceptable salt thereof, decreases other toxicities associated with mucositis, demonstrating the “halo effect” of the therapeutically effective amount of the RRx-001, or a pharmaceutically acceptable salt thereof. RRx-001 may be represented by a formula

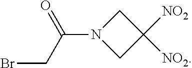 [SMILES: O=C(CBr)N1CC([N+](=O)[O-])([N+](=O)[O-])C1]

### BRIEF DESCRIPTION OF THE DRAWINGS

FIG. 1 is a table showing a trial in which RRx-001 was tested in various groups of patients that were being treated for cancer with a combination of chemotherapy and intensity modulated radiation therapy (IMRT), according to at least some embodiments disclosed herein.

FIG. 2 shows 7 bar graphs. FIG. 2A shows the duration of severe oral mucositis (SOM) in patients for Arms 1, 2, 3, and 4, according to at least some embodiments disclosed herein. FIG. 2B shows a duration of SOM in patients following a last treatment of intensity-modulated radiation therapy (IMRT) for Arms 1, 2, 3, and 4, according to at least some embodiments disclosed herein. FIG. 2C shows a duration of SOM following treatment of 60 gray (Gy) of IMRT for Arms 1, 2, 3, and 4, according to at least some embodiments disclosed herein. FIG. 2D shows a percentage of incidence of SOM following treatment of 60 gray (Gy) of IMRT for Arms 1, 2, 3, and 4, according to at least some embodiments disclosed herein. FIG. 2E shows a percentage of incidence of grade 4 oral mucositis (OM) following treatment of 60 gray (Gy) of IMRT for Arms 1, 2, 3, and 4, according to at least some embodiments disclosed herein. FIG. 2F shows a percentage of patients that resolved SOM during an observation period, according to at least some embodiments disclosed herein. FIG. 2G shows a number of days before onset of SOM in patients for Arms 1, 2, 3, and 4, according to at least some embodiments disclosed herein.

FIG. 3A shows a bar graph showing incidence of grade 4 OM through 60 Gy for Arms 1, 2, 3, and 4 and various therapeutic agents that are available, according to at least some embodiments disclosed herein.

FIG. 3B shows a bar graph showing incidence of SOM through 60 Gy for Arms 1, 2, 3, and 4 and various therapeutic agents that are available, according to at least some embodiments disclosed herein.

FIG. 4 is a bar graph showing duration of SOM after the last treatment of IMRT for Arms 1, 2, 3, 4, and various therapeutic agents that are available, according to at least some embodiments disclosed herein.

FIG. 5 is a Kaplan-Meier survival curve showing a probability of a patient in Arm 4 or collectively Arms 1, 2, or 3 of developing SOM after a start of chemotherapy, according to at least some embodiments disclosed herein.

FIG. 6 is a table showing various incidences of outcomes for patients in Arm 4 of the PREVLAR study vs. patients in Arms 1, 2, and 3 of the PREVLAR study, according to at least some embodiments disclosed herein.

FIG. 7A is a graph showing a Wilcoxon test, which shows a distribution of cumulative cisplatin dose adjusted for Body Surface Area (mg/m²) used Week 1 through Week 7 of the study, according to at least some embodiments disclosed herein.

FIG. 7B is a graph showing a Wilcoxon test of cumulative cisplatin dose adjusted for Body Surface Area (mg/m²) at the end of the study, according to at least some embodiments disclosed herein.

FIG. 8 is a table showing a frequency of various pathogenic toxicities observed in patients in Arm 1, Arm 2, Arm 3, and Arm 4, according to at least some embodiments disclosed herein.

FIG. 9 is a table showing a summary of results related to incidence of SOM for studies comparable to PREVLAR, according to at least some embodiments disclosed herein.

FIG. 10 is a bar graph showing a percentage of cancer recurrence in RRx-001 treated patients, according to at least some embodiments disclosed herein.

FIG. 11 is a table showing a study design for an assessment of RRx-001 for the treatment of oral mucositis induced by acute radiation in hamsters, according to at least some embodiments disclosed herein.

FIG. 12 is a graph of the mean weight change for hamsters in groups 1~4 beginning 4 days before the study and through the study, according to at least some embodiments disclosed herein,

FIG. 13 is a graph of the mean weight change for hamsters in groups 1 and 5-7 beginning 4 days before the study and through the study, according to at least some embodiments disclosed herein.

FIG. 14A shows a graph of mean daily mucositis scores for hamsters in groups 1-4, according to at least some embodiments disclosed herein.

FIG. 14B shows a graph 1450 of mean daily mucositis scores for hamsters in groups 1 and 5-7, according to at least some embodiments disclosed herein.

FIG. 15A is a table showing the number of days in which hamsters in groups 1~4 exhibited an elevated mucositis score of greater than or equal to 3, according to at least some embodiments disclosed herein.

FIG. 15B is a graph of data from the table in FIG. 15A which shows the number of days in which hamsters exhibited an elevated mucositis score of greater than or equal to 3, according to at least some embodiments disclosed herein.

FIG. 16A is a table showing the number of days in which hamsters in groups 1 and 5-7 exhibited an elevated mucositis score of greater than or equal to 3, according to at least some embodiments disclosed herein.

FIG. 16B is a graph of data from the table in FIG. 16A which shows the number of days in which hamsters exhibited an elevated mucositis score of greater than or equal to 3, according to at least some embodiments disclosed herein.

FIG. 17A is a table comparing daily mucositis scores for groups 2-4 with group 1, according to at least some embodiments disclosed herein.

FIG. 17B is a table comparing daily mucositis scores for groups 5-7 with group 1, according to at least some embodiments disclosed herein.

FIG. 18 is a table showing a percent of hamsters with ulceration by day with mucositis scores of greater than or equal to 3, according to at least some embodiments disclosed herein.

FIG. 19 is a chart associated with duration of SOM from baseline to last day of radiation, according to at least some embodiments disclosed herein.

FIG. 20 is a chart comparing an incidence of SOM by study visit, according to at least some embodiments disclosed herein.

FIG. 21 is a graph of estimated severe oral mucositis probabilities according to mixed model repeated measures (MMRM) by study visit and treatment group (pooled RRx-001 as compared to control), according to at least some embodiments disclosed herein.

FIG. 22 is a graph of repeated measures generalized linear mixed-effects model analysis by study cohort, according to at least some embodiments disclosed herein.

FIG. 23 is a graph depicting extreme mouth and throat soreness scores by study week, according to at least some embodiments disclosed herein.

### DETAILED DESCRIPTION

The disclosed subject matter comprises methods and compositions for treating or preventing normal tissue injury from at least one of radiation and chemotherapy in a subject in need thereof. The method comprises administering an effective amount of RRx-001, or a pharmaceutically acceptable salt thereof, to the subject. In some embodiments, the effective amount of RRx-001, or a pharmaceutically acceptable salt thereof, comprises a therapeutically effective amount. In some embodiments, the therapeutically effective amount of the RRx-001, or a pharmaceutically acceptable salt thereof, is administered as a pharmaceutical composition comprising a blood product and/or at least one agent. In some embodiments, the at least one agent is administered prior to, concurrently with or subsequent administration of the therapeutically effective amount of the RRx-001, or a pharmaceutically acceptable salt thereof.

The terms “a” and “an” as used herein mean “one or more” and include the plural unless the context is inappropriate.

Reference to “about” a value or parameter herein includes (and describes) variations that are directed to that value or parameter per se. In embodiments, the term “about” refers to +/−10%, +/−5%, or +/−1%, of the designated value.

The “comprise” or variations such as “comprises” or “comprising,” will be understood to imply the inclusion of a stated element, integer or step, or group of elements, integers or steps, but not the exclusion of any other element, integer or step, or group of elements, integers or steps. Embodiments described herein also include “consisting” and/or “consisting essentially of” aspects.

“Treatment”, “treating” or similar phrases refer to obtaining beneficial or desired results, such as clinical results, for a subject, suffering from a condition or disorder, such as the normal tissue injury from the at least one of radiation and chemotherapy. As used herein, the term “treating” includes any effect, e.g., lessening, reducing, modulating, ameliorating or eliminating, that results in the improvement of the condition, disorder, and the like, such as the normal tissue injury, or ameliorating a symptom thereof. Beneficial or desired results include any one or more of: alleviating one or more symptoms, diminishing the extent of the normal tissue injury, and improving quality of life.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route.

As used herein, the term “subject” or “patient” refers to an organism to be treated by the methods of the present invention. Such organisms are preferably mammals (e.g., murines, simians, equines, bovines, porcines, canines, felines, and the like), and more preferably humans. In some embodiments, the human subject comprises an athlete or military personnel. In some embodiments, the subject is a neonate (i.e., less than one month old), an infant (at least one month to one year old) or a toddler (one year to three years old). In other embodiments, the subject is a child (three to 18 years old). In still other embodiments, the subject is an adult (≥18 years old).

The phrase “parenteral administration” and “administered parenterally” as used herein means modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

As used herein, the term “composition” or “pharmaceutical composition” refers to the combination of an active agent with an excipient or a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, PA [1975]).

As used herein, the term “pharmaceutically acceptable salt” refers to any circular salt (e.g., acid or base) of a compound of the present invention suitable for pharmaceutical administration which, upon administration to the subject, is capable of providing a compound of this invention or an active metabolite or residue thereof. As is known to those of skill in the art, “salts” of the compounds of the present invention may be derived from inorganic or organic acids and bases.

Examples of acids include, but are not limited to, hydrochloric, hydrobromic, sulfuric, nitric, perchloric, fumaric, maleic, phosphoric, glycolic, lactic, salicylic, succinic, toluene psulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW⁴⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH⁴⁺, and NW⁴⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

RRx-001 (also called ABDNAZ), with the chemical name 2-bromo-1-(3,3-dinitroazetidin-1-yl) ethan-1-one, is a small cyclic nitro compound in a Phase 3 clinical trial for the treatment of cancer. It has the following structure:

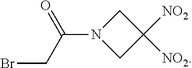 [SMILES: O=C(CBr)N1CC([N+](=O)[O-])([N+](=O)[O-])C1]

It has been shown by studies from multiple independent groups that RRx-001 is safe and well-tolerated in humans. Additionally, no dose-limiting toxicities and no drug-drug interactions have been observed. See N. Jayabalan, et al. (2023) Drugs 83(5):389-402. Methods of synthesizing ABDNAZ have been described, such as in U.S. Pat. Nos. 7,507,842 and 8,471,041.

In any of the embodiments herein, RRx-001 may be in a salt or non-salt form. Furthermore, the RRx-001 or salt thereof may be in the form of a hydrate, solvate, co-crystal, clathrate, or other complexed form.

Its anticancer effects are complemented, paradoxically, by antioxidant and anti-inflammatory properties that protect normal tissues but not tumors from the toxicities of chemotherapy and radiation. In established hamster oral mucositis (OM) models induced by a single dose of radiation, RRx-001 protected against and sped recovery from mucosal injury. In a randomized 4 arm Phase 2 trial called PREVLAR (NCT 03515538), which compared the anti-mucositis activity and safety of 3 different dosing schedules of RRx-001 with standard-of-care (SOC) cisplatin and intensity modulated radiation therapy (IMRT), severe oral mucositis (SOM) among RRx-001-treated patients given 3 different treatment schedules was less severe, of shorter duration, demonstrated delayed time to onset and resolved earlier. Overall, the incidence, duration and severity of SOM and oropharyngeal dysphagia were reduced. Moreover, a greater number of complete responses (CRs) and partial responses (PRs) was seen on the RRx-001-treated arms compared to SOC, which may correlate with a higher relative dose intensity (RDI). Dose intensity (DI) is the total amount of drug delivered over the total time course of treatment. Relative dose intensity (RDI) is the ratio of the dose intensity delivered to the reference standard dose intensity for a chemotherapy regimen, in this case cisplatin. RDI for chemotherapy is strongly correlated with improved response rates and overall survival; results from the PREVLAR study indicate that RRx-001-treated patients experienced a lower incidence of oropharyngeal toxicities and, as a result, received greater cisplatin dose-intensity relative to control, which is hypothesized to have led not only to a greater number of CRs and PRs on the RRx-001-treated arms but also a lower incidence of cancer recurrence.

In summary, RRx-001 was shown in the PREVLAR trial to act synergistically with radiation or chemotherapy to limit tumor cell growth while concurrently protecting and preserving the integrity of the oral mucosal barrier function through its antioxidant and anti-inflammatory effects in non-cancer cells. The only other FDA approved anti-mucositis agent, palifermin, is approved for use only in bone marrow transplantation not in head and neck cancer, because of suspected tumor growth promoting properties. Unlike palifermin and other anti-mucositis agents in development, RRx-001 is shown not to interfere with the therapeutic effects of chemoradiation (CRT) in the treatment of head and neck tumors, or to promote growth of cancer cells, but instead sensitizes tumor cells to CRT. In various embodiments, tumor ablation is increased when a patient is administered RRx-001 concurrently with chemotherapy and/or radiation treatment.

The disclosed subject matter, which is based on the paradoxical discovery that RRx-001 has both anti-mucositis and anti-head and neck cancer properties, simultaneously protecting against the toxic effects of chemoradiation while enhancing cytotoxicity against malignant cells, relates to methods for administering a prophylactically or therapeutically effective amount of RRx-001. The method includes adjunctive administration of RRx-001 in various ways such as orally via “swish and spit” or intravenously or direct intratumoral injection in conjunction with, before, or after administration of chemotherapy and/or radiation. The reduction of toxicity to normal cells is postulated to allow for more effective treatment, resulting from the use of higher doses, more prolonged treatment and use of drugs otherwise deemed too toxic especially in vulnerable elderly or pediatric populations. In some embodiments, the disclosed subject matter inhibits or decreases apoptosis in normal cells and tissues due to therapies such as chemotherapy and radiation. In some embodiments, the use of RRx-001 permits a selective increase in the concentration of antioxidants, for example, glutathione in healthy tissue.

In one embodiment, a cytotoxic agent that is used in conjunction with RRx-001 is a cancer chemotherapeutic agent. The cytotoxic agent is dose-limited due to the development of adverse side effects such as mucositis and esophagitis. The cancer chemotherapeutic agent is selected from the group consisting of platinum derivatives such as cisplatin and carboplatin, taxanes (e.g., paclitaxel and docetaxel), steroid derivatives, PARP inhibitors, anti-metabolites (e.g., 5-fluorouracil, methotrexate, gemcitabine, 6-mercaptopurine), vinca alkaloids, antitumor antibiotics (e.g., bleomycin, mitomycin adriamycin and doxorubicin), checkpoint inhibitors, oncolytic viruses, EGFR inhibitors (e.g., cetuximab), tyrosine kinase inhibitors, epigenetic inhibitors (e.g., HDACs and DNA methyltransferase inhibitors), etoposide, arsenic derivatives, intercalating agents, alkylating agents (e.g., melphalan and cyclophosphamide) and combinations thereof. The cytotoxic agent is administered within 24 hours (before, during or after) of RRx-001 administration. In yet another embodiment, RRx-001 is administered within 24 hours (before, during or after) of a thiol-based chemoradioprotectant agent, for example, N-acetyl cysteine (NAC), amifostine, sodium thiosulfate (STS), D-methionine, GSH ethyl ester and GlyNAC ((Glycine and N-Acetylcysteine).

An additional advantage of RRx-001 is that it is thought to be protective against the adverse effects of nausea/vomiting and hypotension that are common to thiol-based chemoradioprotectants like amifostine. In yet another embodiment, RRx-001 is administered within 24 hours (before, during or after) of another agent that is also used to protect, prevent, or manage the development of oral mucositis. These agents include, without limitation, GM-CSF, palifermin, pilocarpine, gabapentin, keratinocyte growth factor (KGF), coconut oil, MucoLox, cetuximab, a checkpoint inhibitor, transforming growth factor beta (TGF-β), an epidermal growth factor, honey, benzydamine, a corticosteroid, rebamipide, an antimicrobial agent, misoprostol, Amifostine, brilacidin, L-glutamine, low-level laser therapy, laser and light therapy, cryotherapy, vitamin E, pentoxyfyilline, GC4419, clonidine, melatonin, a probiotic, actovegin, aloe vera, allopurinol, azithromycin, Black Mulberry Molasses, Glycerin payayor, Qingre Liyan decoction, erythropoietin mouthwash, EC-18, sucralfate, Avasopasem manganese, and lidocaine. In some embodiments, the checkpoint inhibitor is selected from the group consisting of: pembrolizumab and nivolumab. In some embodiments, the antimicrobial agent is selected from the group consisting of: chlorhexidine, polymyxin E, and amphotericin.

In various embodiments, the treatment of RRx-001 is administered to a patient that has a locally advanced solid tumor. Examples of locally advanced solid tumors include but are not limited to gastrointestinal malignancies, head and neck cancers, gynecological cancers, lung cancers, breast cancers, hepatocellular cancer, esophageal cancer, genitourinary cancers, glioblastoma, and sarcoma.

### Methods of Administering RRX-001

The RRx-001, or a pharmaceutically acceptable salt thereof (e.g., a therapeutically effective amount), may be administered to a patient in various dosages, at various frequencies, over various periods of time that correspond to administration of various therapeutic agents. The term patient, when used herein, refers to a human or animal subject that is being treated for a condition. In an exemplary embodiment, the patient is a human being that is being treated for cancer with therapeutic agents that are known to cause one or more adverse side effects. In an exemplary embodiment, the patient is being treated with a combination of chemotherapeutic agents and radiation therapy.

In various embodiments, the RRx-001, or a pharmaceutically acceptable salt thereof (e.g., a therapeutically effective amount), is administered via inhalation, nasal administration, topical administration, oral administration, transdermal administration, intra-aural administration, rectal administration, intravenous administration, intramuscular administration, subcutaneous administration, intraperitoneal administration, or combinations thereof. In various embodiments, RRx-001 is administered by intravenous injection. In an exemplary embodiment, RRx-001 is mixed ex-vivo with blood before being administered via intravenous injection. In various embodiments, RRx-001 is administered by oral administration or swish and spit or swish and swallow. Swish and spit refers to a patient taking a solution containing RRx-001 into their mouth, orally swishing the solution, and spitting out the solution. Swish and swallow comprises the patient orally swishing the RRx-001 solution before swallowing the RRx-001 solution. In various embodiments, RRx-001 is administered by direct intratumoral injection.

An example of oral administration comprises the subject taking the RRx-001, or a pharmaceutically acceptable salt thereof (e.g., a therapeutically effective amount), by mouth. Transdermal administration comprises administering the RRx-001 through the skin of the subject. An example of transdermal administration includes applying a composition containing the RRx-001, or a pharmaceutically acceptable salt thereof (e.g., a therapeutically effective amount), that permeates the skin and delivers the RRx-001. Intra-aural administration comprises administering the RRx-001, or a pharmaceutically acceptable salt thereof (e.g., a therapeutically effective amount), through the ear. An example of intra-aural administration may include ear drops for the subject that contain an amount of the RRx-001, or a pharmaceutically acceptable salt thereof (e.g., a therapeutically effective amount). Rectal administration comprises administration of the RRx-001, or a pharmaceutically acceptable salt thereof (e.g., a therapeutically effective amount), through the anus of the subject. An example of rectal administration comprises administering a suppository that contains an amount of the RRx-001 to the subject.

Intravenous administration comprises injecting the RRx-001, or a pharmaceutically acceptable salt thereof (e.g., a therapeutically effective amount), directly into a vein of the subject and may include a combination with a sample of autologous or allogenic blood from a compatible donor. Intramuscular administration comprises injecting a composition that includes the RRx-001, or a pharmaceutically acceptable salt thereof (e.g., a therapeutically effective amount), directly into a muscle of the subject. Subcutaneous administration comprises delivering the RRx-001, or a pharmaceutically acceptable salt thereof (e.g., a therapeutically effective amount), just under a layer of skin. An example of subcutaneous administration comprises injecting a composition including the RRx-001, or a pharmaceutically acceptable salt thereof (e.g., a therapeutically effective amount), into a layer a fatty tissue just under the skin of the subject. Intraperitoneal administration comprises injecting the RRx-001, or a pharmaceutically acceptable salt thereof (e.g., a therapeutically effective amount), into the peritoneum of the subject.

In various embodiments, the RRx-001, or a pharmaceutically acceptable salt thereof (e.g., a therapeutically effective amount), is administered in combination with one or more other agents. The term “in combination” when used herein, may refer to administering two or more agents concurrently or in sequence. Concurrent administration may refer to administering two agents at approximately the same time. Concurrent administration may refer to administering an agent at regular intervals where the regular intervals overlap with administration of one or more other agents. Sequential administration may refer to administering two or more agents at different times, but such that the two or more agents result in a combined effect on the subject. In various embodiments, the other agent is selected from the group consisting of: Dexamethasone, Erbitux (Cetuximab), Avasopasem (GC4419), Pembrolizumab, Nivolumab, Hydrocortisone, Prednisone, Cyclophosphamide, Methotrexate, Paclitaxel, Carboplatin, Etoposide, Gemcitabine, Cisplatin, Oxaliplatin, Chlorambucil, Mechloethamine, Melphalan, Tocilizumab, Brentuximab Vedotin, Doxorubicin, Afatinib, Everolimus, Netupitant, Palonosetron, Imiquimod, and Fluorouracil. In some embodiments, the agent is selected from the group consisting of: a PARP inhibitor, a tyrosine kinase inhibitor, a thiol-based chemoradioprotectant agent, an EGFR inhibitor, an HDAC inhibitor, a DNA methyltransferase inhibitor, 5-FU, imatinib, hydroxyurea, taxol, an oncolytic virus, a checkpoint inhibitor, and a topoisomerase inhibitor. Non-limiting examples of the thiol-based chemoradioprotectant agent include NAC, amifostine, STS, D-methionine, GSH ethyl ester, and GlyNAC. Non-limiting examples of the topoisomerase inhibitor comprise irinotecan and doxorubicin. In some examples, the other agent treats oral mucositis and is selected from the group consisting of: GM-CSF, palifermin, pilocarpine, gabapentin, keratinocyte growth factor (KGF), coconut oil, MucoLox, cetuximab, a checkpoint inhibitor, transforming growth factor beta (TGF-β), an epidermal growth factor, honey, benzydamine, a corticosteroid, rebamipide, an antimicrobial agent, misoprostol, Amifostine, brilacidin, L-glutamine, low-level laser therapy, laser and light therapy, cryotherapy, vitamin E, pentoxyfyilline, GC4419, clonidine, melatonin, a probiotic, actovegin, aloe vera, allopurinol, azithromycin, Black Mulberry Molasses, Glycerin payayor, Qingre Liyan decoction, erythropoietin mouthwash, EC-18, sucralfate, Avasopasem manganese, and lidocaine. In some embodiments, the checkpoint inhibitor is selected from the group consisting of: pembrolizumab and nivolumab. In some embodiments, the antimicrobial agent is selected from the group consisting of: chlorhexidine, polymyxin E, and amphotericin. In some embodiments, administering the other agent prior to, concurrently with, or subsequent administering the effective amount of RRx-001, or a pharmaceutically acceptable salt thereof (e.g., a therapeutically effective amount), prevents or reduces at least one side effect associated with the agent.

In some embodiments, administering the effective amount of the RRx-001, or a pharmaceutically acceptable salt thereof (e.g., a therapeutically effective amount), increases tumor ablation in the subject. In some embodiments, administering the effective amount of the RRx-001, or a pharmaceutically acceptable salt thereof (e.g., a therapeutically effective amount), attenuates secondary cancer development in the subject. In some embodiments, administering the effective amount of the RRx-001, or a pharmaceutically acceptable salt thereof (e.g., a therapeutically effective amount), selectively protects normal tissue from an injury relative to tumor tissue in the subject. In some embodiments, the injury is selected from the group consisting of: mucositis, dysphagia, dyspepsia, laryngeal inflammation, oral dysesthesia, vomiting, salivary duct inflammation, esophagitis, any gastrointestinal distress, myelosuppression, impotence, infertility, dermatitis, hair loss and increased creatinine. In some embodiments, administering the effective amount of the RRx-001, or a pharmaceutically acceptable salt thereof (e.g., a therapeutically effective amount), results in the subject experiencing a decrease in malnutrition. In some embodiments, administering the effective amount of the RRx-001, or a pharmaceutically acceptable salt thereof (e.g., a therapeutically effective amount), results in the subject having a lower incidence of gastrostomy-tube placement. In some embodiments, administering the effective amount of the RRx-001, or a pharmaceutically acceptable salt thereof (e.g., a therapeutically effective amount), results in the subject having a lower incidence of weight loss.

Toxicities associated with cancer therapy typically occur in clusters. It is rare for patients to develop only one side effect. In the case of concomitant chemoradiation for head and neck cancers, in addition to mucositis, patients are prone to damage to the parotid glands and ducts and radiation-induced dermatitis with consequent xerostomia, dysphagia, weight loss, and increased risk of candidiasis. The pathobiological basis for these changes is shared with those for mucositis. As such, in some embodiments, administration of the therapeutically effective amount of the RRx-001, or a pharmaceutically acceptable salt thereof, decreases other toxicities associated with mucositis, demonstrating the “halo effect” of the therapeutically effective amount of the RRx-001, or a pharmaceutically acceptable salt thereof.

In some embodiments, administering the effective amount of the RRx-001, or a pharmaceutically acceptable salt thereof (e.g., a therapeutically effective amount), increases a tolerated dose of the at least one of the radiation and the chemotherapy in treating the human subject for cancer. In some embodiments, the chemotherapy is platinum-based chemotherapy. In some embodiments, the platinum-based chemotherapy is selected from the group consisting of: cisplatin, oxaliplatin, carboplatin, nedaplatin, heptaplatin, and lobaplatin. In some embodiments, wherein increasing the tolerated dose of the at least one of the radiation and the chemotherapy in treating the human subject for cancer comprises increasing a previously poorly tolerated dose of the at least one of the radiation and the chemotherapy.

In some embodiments, a maximum or increased tolerated amount or RDI of the at least one of the radiation and the chemotherapy increases subsequent to administering the effective amount of the RRx-001, or a pharmaceutically acceptable salt thereof (e.g., a therapeutically effective amount). In some embodiments, the previously poorly tolerated dose of the at least one of the radiation and the chemotherapy becomes tolerable subsequent to administering the effective amount of the RRx-001, or a pharmaceutically acceptable salt thereof (e.g., a therapeutically effective amount). In some embodiments, the maximum or increased tolerated amount of the at least one of the radiation and the chemotherapy comprises administering an increased dose of at least one of cisplatin and radiotherapy each time the at least one of the cisplatin and the radiotherapy are administered. In some embodiments, the maximum or increased tolerated amount of the cisplatin each time the cisplatin is administered is in a range between about 50 mg and about 150 mg. In some embodiments, administering the effective amount of the RRx-001, or a pharmaceutically acceptable salt thereof (e.g., a therapeutically effective amount), results in an increased anti-tumor efficacy of the at least one of the cisplatin and the radiotherapy in a treatment for cancer.

### Dosage

The disclosed subject matter are methods of treatment that include administering the RRx-001. In various embodiments, the RRx-001 is administered to a patient or the subject prior to and/or concurrently with one or more therapeutic agents. In various embodiments, the RRx-001 may be administered as the RRx-001, or a pharmaceutically acceptable salt thereof (e.g., a therapeutically effective amount). The RRx-001 may be administered in various amounts. The dosages provided herein refer to the amount of the RRx-001, excluding the weight of any counterion that may be present. The amount may refer to a total amount of the RRx-001 that is administered over a period of time or a dosage of individual administrations of the RRx-001. In various embodiments, the RRx-001, or a pharmaceutically acceptable salt thereof (e.g., a therapeutically effective amount), may be administered in one or more doses of between about 0.1 mg and about 1000 mg. In various embodiments, the RRx-001, or a pharmaceutically acceptable salt thereof (e.g., a therapeutically effective amount), may be administered in one or more doses of between about 0.5 mg and about 500 mg. In various embodiments, the RRx-001, or a pharmaceutically acceptable salt thereof (e.g., a therapeutically effective amount), may be administered in one or more doses of between about 1 mg and about 100 mg. In various embodiments, the RRx-001, or a pharmaceutically acceptable salt thereof (e.g., a therapeutically effective amount), may be administered in one or more doses of between about 5 mg and about 50 mg. In various embodiments, the RRx-001, or a pharmaceutically acceptable salt thereof (e.g., a therapeutically effective amount), may be administered in one or more doses of between about 0.5 mg and about 200 mg. In various embodiments, the RRx-001, or a pharmaceutically acceptable salt thereof (e.g., a therapeutically effective amount), may be administered in one or more doses of between about 0.5 mg and about 100 mg. In various embodiments, the RRx-001, or a pharmaceutically acceptable salt thereof (e.g., a therapeutically effective amount), may be administered in one or more doses of between about 0.5 mg and about 50 mg.

In various embodiments, the RRx-001, or a pharmaceutically acceptable salt thereof (e.g., a therapeutically effective amount), may be administered in one or more doses of between about 1 mg and about 50 mg. In various embodiments, the RRx-001, or a pharmaceutically acceptable salt thereof (e.g., a therapeutically effective amount), may be administered in one or more doses of between about 10 mg and about 50 mg. In various embodiments, the RRx-001, or a pharmaceutically acceptable salt thereof (e.g., a therapeutically effective amount), may be administered in one or more doses of between about 15 mg and about 50 mg. In various embodiments, the RRx-001, or a pharmaceutically acceptable salt thereof (e.g., a therapeutically effective amount), may be administered in one or more doses of between about 20 mg and about 50 mg. In various embodiments, the RRx-001, or a pharmaceutically acceptable salt thereof (e.g., a therapeutically effective amount), may be administered in one or more doses of between about 25 mg and about 50 mg. In various embodiments, the RRx-001, or a pharmaceutically acceptable salt thereof (e.g., a therapeutically effective amount), may be administered in one or more doses of between about 30 mg and about 50 mg. In various embodiments, the RRx-001, or a pharmaceutically acceptable salt thereof (e.g., a therapeutically effective amount), may be administered in one or more doses of between about 35 mg and about 50 mg. In various embodiments, the RRx-001, or a pharmaceutically acceptable salt thereof (e.g., a therapeutically effective amount), may be administered in one or more doses of between about 40 mg and about 50 mg. In various embodiments, the RRx-001, or a pharmaceutically acceptable salt thereof (e.g., a therapeutically effective amount), may be administered in one or more doses of between about 45 mg and about 50 mg.

In various embodiments, the RRx-001, or a pharmaceutically acceptable salt thereof (e.g., a therapeutically effective amount), may be administered in one or more doses of between about 1 mg and about 2 mg. In various embodiments, the RRx-001, or a pharmaceutically acceptable salt thereof (e.g., a therapeutically effective amount), may be administered in one or more doses of between about 2 mg and about 3 mg. In various embodiments, the RRx-001, or a pharmaceutically acceptable salt thereof (e.g., a therapeutically effective amount), may be administered in one or more doses of between about 3 mg and about 4 mg. In various embodiments, the RRx-001, or a pharmaceutically acceptable salt thereof (e.g., a therapeutically effective amount), may be administered in one or more doses of between about 4 mg and about 5 mg. In various embodiments, the RRx-001, or a pharmaceutically acceptable salt thereof (e.g., a therapeutically effective amount), may be administered in one or more doses of between about 5 mg and about 6 mg. In various embodiments, the RRx-001, or a pharmaceutically acceptable salt thereof (e.g., a therapeutically effective amount), may be administered in one or more doses of between about 7 mg and about 8 mg. In various embodiments, the RRx-001, or a pharmaceutically acceptable salt thereof (e.g., a therapeutically effective amount), may be administered in one or more doses of between about 8 mg and about 9 mg. In various embodiments, the RRx-001, or a pharmaceutically acceptable salt thereof (e.g., a therapeutically effective amount), may be administered in one or more doses of between about 9 mg and about 10 mg. In various embodiments, the RRx-001, or a pharmaceutically acceptable salt thereof (e.g., a therapeutically effective amount), may be administered in one or more doses of between about 10 mg and about 15 mg. In various embodiments, the RRx-001, or a pharmaceutically acceptable salt thereof (e.g., a therapeutically effective amount), may be administered in one or more doses of between about 10 mg and about 15 mg. In various embodiments, the RRx-001, or a pharmaceutically acceptable salt thereof (e.g., a therapeutically effective amount), may be administered in one or more doses of between about 15 mg and about 20 mg. In various embodiments, the RRx-001, or a pharmaceutically acceptable salt thereof (e.g., a therapeutically effective amount), may be administered in one or more doses of between about 20 mg and about 25 mg. In various embodiments, the RRx-001, or a pharmaceutically acceptable salt thereof (e.g., a therapeutically effective amount), may be administered in one or more doses of between about 25 mg and about 30 mg. In various embodiments, the RRx-001, or a pharmaceutically acceptable salt thereof (e.g., a therapeutically effective amount), may be administered in one or more doses of between about 30 mg and about 35 mg. In various embodiments, the RRx-001, or a pharmaceutically acceptable salt thereof (e.g., a therapeutically effective amount), may be administered in one or more doses of between about 35 mg and about 40 mg. In various embodiments, the RRx-001, or a pharmaceutically acceptable salt thereof (e.g., a therapeutically effective amount), may be administered in one or more doses of between about 40 mg and about 45 mg. In various embodiments, the RRx-001, or a pharmaceutically acceptable salt thereof (e.g., a therapeutically effective amount), may be administered in one or more doses of between about 45 mg and about 50 mg. In various embodiments, the RRx-001, or a pharmaceutically acceptable salt thereof (e.g., a therapeutically effective amount), may be administered in one or more doses of between about 50 mg and about 55 mg. In various embodiments, the RRx-001, or a pharmaceutically acceptable salt thereof (e.g., a therapeutically effective amount), may be administered in one or more doses of between about 55 mg and about 60 mg. In various embodiments, the RRx-001, or a pharmaceutically acceptable salt thereof (e.g., a therapeutically effective amount), may be administered in one or more doses of between about 60 mg and about 65 mg. In various embodiments, the RRx-001, or a pharmaceutically acceptable salt thereof (e.g., a therapeutically effective amount), may be administered in one or more doses of between about 65 mg and about 70 mg. In various embodiments, the RRx-001, or a pharmaceutically acceptable salt thereof (e.g., a therapeutically effective amount), may be administered in one or more doses of between about 70 mg and about 75 mg. In various embodiments, the RRx-001, or a pharmaceutically acceptable salt thereof (e.g., a therapeutically effective amount), may be administered in one or more doses of between about 75 mg and about 80 mg. In various embodiments, the RRx-001, or a pharmaceutically acceptable salt thereof (e.g., a therapeutically effective amount), may be administered in one or more doses of between about 80 mg and about 85 mg. In various embodiments, the RRx-001, or a pharmaceutically acceptable salt thereof (e.g., a therapeutically effective amount), may be administered in one or more doses of between about 85 mg and about 90 mg. In various embodiments, the RRx-001, or a pharmaceutically acceptable salt thereof (e.g., a therapeutically effective amount), may be administered in one or more doses of between about 90 mg and about 95 mg. In various embodiments, the RRx-001, or a pharmaceutically acceptable salt thereof (e.g., a therapeutically effective amount), may be administered in one or more doses of between about 95 mg and about 100 mg. In various embodiments, the RRx-001, or a pharmaceutically acceptable salt thereof (e.g., a therapeutically effective amount), may be administered in one or more doses of between about 100 mg and about 110 mg. In various embodiments, the RRx-001, or a pharmaceutically acceptable salt thereof (e.g., a therapeutically effective amount), may be administered in one or more doses of between about 100 mg and about 110 mg. In various embodiments, the RRx-001, or a pharmaceutically acceptable salt thereof (e.g., a therapeutically effective amount), may be administered in one or more doses of between about 110 mg and about 120 mg. In various embodiments, the RRx-001, or a pharmaceutically acceptable salt thereof (e.g., a therapeutically effective amount), may be administered in one or more doses of between about 120 mg and about 130 mg. In various embodiments, the RRx-001, or a pharmaceutically acceptable salt thereof (e.g., a therapeutically effective amount), may be administered in one or more doses of between about 130 mg and about 140 mg. In various embodiments, the RRx-001, or a pharmaceutically acceptable salt thereof (e.g., a therapeutically effective amount), may be administered in one or more doses of between about 140 mg and about 150 mg. In various embodiments, the RRx-001, or a pharmaceutically acceptable salt thereof (e.g., a therapeutically effective amount), may be administered in one or more doses of between about 150 mg and about 160 mg. In various embodiments, the RRx-001, or a pharmaceutically acceptable salt thereof (e.g., a therapeutically effective amount), may be administered in one or more doses of between about 160 mg and about 200 mg. In various embodiments, the RRx-001, or a pharmaceutically acceptable salt thereof (e.g., a therapeutically effective amount), may be administered in one or more doses of between about 160 mg and about 170 mg. In various embodiments, the RRx-001, or a pharmaceutically acceptable salt thereof (e.g., a therapeutically effective amount), may be administered in one or more doses of between about 170 mg and about 180 mg. In various embodiments, the RRx-001, or a pharmaceutically acceptable salt thereof (e.g., a therapeutically effective amount), may be administered in one or more doses of between about 180 mg and about 190 mg. In various embodiments, the RRx-001, or a pharmaceutically acceptable salt thereof (e.g., a therapeutically effective amount), may be administered in one or more doses of between about 190 mg and about 200 mg.

### Period of Treatment

The doses of the RRx-001, or a pharmaceutically acceptable salt thereof (e.g., a therapeutically effective amount), may be administered over various periods of time at various intervals. In various embodiments, the RRx-001, or a pharmaceutically acceptable salt thereof (e.g., a therapeutically effective amount), is administered during a pretreatment period before administering at least one therapeutic agent to a subject or a patient. In various embodiments, the RRx-001, or a pharmaceutically acceptable salt thereof (e.g., a therapeutically effective amount), is administered concurrently with administration of the at least one therapeutic agent to the subject or the patient. In various embodiments, the RRx-001, or a pharmaceutically acceptable salt thereof (e.g., a therapeutically effective amount), is administered after administration of the at least one therapeutic agent to the subject or the patient. In various embodiments, the RRx-001, or a pharmaceutically acceptable salt thereof (e.g., a therapeutically effective amount), is administered in combinations of before, during and after administration of the at least one therapeutic agent to the subject or the patient.

In an exemplary embodiment, the pretreatment period is between about 1 day and about 1 year. In an exemplary embodiment, the pretreatment period is between about 2 days and about 3 months. In an exemplary embodiment, the pretreatment period is between about 3 days and about 2 months. In an exemplary embodiment, the pretreatment period is between about 5 days and about 1 month. In an exemplary embodiment, the pretreatment period is between about 1 week and about 3 weeks. In an exemplary embodiment, the pretreatment period is between about 10 days and about 20 days. In an exemplary embodiment, the pretreatment period is about 2 weeks.

In an exemplary embodiment, the doses of the RRx-001, or a pharmaceutically acceptable salt thereof (e.g., a therapeutically effective amount), are administered during a treatment with at least one therapeutic agent. The treatment period of the at least one therapeutic agent, for the purpose of this disclosure, is considered to be a period of time that a patient is administered one or more therapeutic agents at regular intervals. For example, a treatment of the at least one therapeutic agent may comprise X administrations of a therapeutic agent per week for a period of Y weeks. The RRx-001, or a pharmaceutically acceptable salt thereof (e.g., a therapeutically effective amount), may be administered before, after, or during administration of the at least one therapeutic agent.

In an exemplary embodiment, the at least one therapeutic agent is administered over a period of between about 1 day and about 1 year. The therapeutic agent may comprise one or more agents that treat a condition. In an exemplary embodiment, the at least one therapeutic agent comprises a chemotherapeutic agent and radiation therapy. Where the therapeutic agent comprises more than one agent, two or more agents may be administered during that same time period, different time periods, or overlapping time periods. Where the time periods of the various agents are not identical, the more than one time period may be identified as a first time period, a second time period, and so on where each time period corresponds to the time that each agent is administered. The RRx-001, or a pharmaceutically acceptable salt thereof (e.g., a therapeutically effective amount), may be administered before, during, or after any of the various time periods. In an exemplary embodiment, the RRx-001, or a pharmaceutically acceptable salt thereof (e.g., a therapeutically effective amount), is administered during a first time period only. In an exemplary embodiment, the RRx-001, or a pharmaceutically acceptable salt thereof (e.g., a therapeutically effective amount), is administered during a second time period only. In an example of various embodiments, if a chemotherapeutic agent is administered during a first time period and IMRT is administered during a second time period, the RRx-001, or a pharmaceutically acceptable salt thereof (e.g., a therapeutically effective amount), may be administered concurrently with the first time period, second time period, both first time period and second time period, in between the first time period and second time period (if applicable), during an overlap between the first time period and second time period (if applicable), or during a portion of the periods listed herein. The RRx-001, or a pharmaceutically acceptable salt thereof (e.g., a therapeutically effective amount), may be administered over various combinations of the time periods. In an example where the therapeutic agent comprises three agents that are administered over three time periods, the RRx-001, or a pharmaceutically acceptable salt thereof (e.g., a therapeutically effective amount), may be administered during a combination of the first and second time period, the first and third time period, or the second and third time period.

In various embodiments, the RRx-001, or a pharmaceutically acceptable salt thereof (e.g., a therapeutically effective amount), is administered in different dosage amounts and/or different frequencies over the different time periods. In an exemplary embodiment, the RRx-001, or a pharmaceutically acceptable salt thereof (e.g., a therapeutically effective amount), is administered as a first dosage over a first time period and as a second dosage over a second time period. Similarly, in an exemplary embodiment, the RRx-001, or a pharmaceutically acceptable salt thereof (e.g., a therapeutically effective amount), may be administered a dosage at a first frequency over a first time period and a dosage at a second frequency over a second time period. A dosage amount may vary between time periods. In one example of an embodiment that changes a dosage, a first dosage is administered at a frequency during a first time period and a second dosage is administered at a frequency during a second time period.

### Frequency of Administration

Each dose of RRx-001 may be administered at various intervals over a time period. The various intervals are referred to herein as a “frequency” or “frequency of administration.” In an exemplary embodiment, each dose of the RRx-001, or a pharmaceutically acceptable salt thereof (e.g., a therapeutically effective amount), is administered at a frequency of between about 1 dose per hour and about 1 dose per year until the end of a dosing period. In an exemplary embodiment, each dose of the RRx-001, or a pharmaceutically acceptable salt thereof (e.g., a therapeutically effective amount), is administered at a frequency of between about 1 time per hour to about 1 time per 90 days during a dosing period. In an exemplary embodiment, each dose of the RRx-001, or a pharmaceutically acceptable salt thereof (e.g., a therapeutically effective amount), is administered at a frequency of between about 1 time per hour to about 1 time per 75 days during a dosing period. In an exemplary embodiment, each dose of the RRx-001, or a pharmaceutically acceptable salt thereof (e.g., a therapeutically effective amount), is administered at a frequency of between about 1 time per hour to about 1 time per 60 days during a dosing period. In an exemplary embodiment, each dose of the RRx-001, or a pharmaceutically acceptable salt thereof (e.g., a therapeutically effective amount), is administered at a frequency of between about 1 time per 2 hours to about 1 time per 45 days during a dosing period. In an exemplary embodiment, each dose of the RRx-001, or a pharmaceutically acceptable salt thereof (e.g., a therapeutically effective amount), is administered at a frequency of between about 1 time per 6 hours to about 1 time per 30 days during a dosing period. In an exemplary embodiment, each dose of the RRx-001, or a pharmaceutically acceptable salt thereof (e.g., a therapeutically effective amount), is administered at a frequency of between about 1 time per day to about 1 time per 3 weeks during a dosing period. In an exemplary embodiment, each dose of the RRx-001, or a pharmaceutically acceptable salt thereof (e.g., a therapeutically effective amount), is administered at a frequency of between about 2 times per week to about 1 time per 3 weeks during a dosing period. In an exemplary embodiment, each dose of the RRx-001, or a pharmaceutically acceptable salt thereof (e.g., a therapeutically effective amount), is administered at a frequency of between about 1 time per 5 days to about 1 time per 2 weeks during a dosing period. In an exemplary embodiment, each dose of the RRx-001, or a pharmaceutically acceptable salt thereof (e.g., a therapeutically effective amount), is administered at a frequency of between about 1 time per week to about 1 time per 10 days during a dosing period.

In various embodiments, a dosage of the RRx-001, or a pharmaceutically acceptable salt thereof (e.g., a therapeutically effective amount), administration and a frequency of the RRx-001, or a pharmaceutically acceptable salt thereof (e.g., a therapeutically effective amount), administration may be adjusted indirectly proportionally such that a cumulative amount or DI of the RRx-001, or a pharmaceutically acceptable salt thereof (e.g., a therapeutically effective amount), that is administered remains substantially unchanged. For example, a dosage of the RRx-001, or a pharmaceutically acceptable salt thereof (e.g., a therapeutically effective amount), may be increased as a frequency of administration decreases. For the purposes of this disclosure, the cumulative amount of the RRx-001, or a pharmaceutically acceptable salt thereof (e.g., a therapeutically effective amount), that is administered to a patient may be a total amount of the RRx-001, or a pharmaceutically acceptable salt thereof (e.g., a therapeutically effective amount), that is administered over a time period for all disclosed dosage amounts and all disclosed frequencies.

In various embodiments, the RRx-001, or a pharmaceutically acceptable salt thereof (e.g., a therapeutically effective amount), is administered in a limited dose that precludes further administration of the RRx-001, or a pharmaceutically acceptable salt thereof (e.g., a therapeutically effective amount). The reason for the limited dose is because results suggest that some effects of the RRx-001, or a pharmaceutically acceptable salt thereof (e.g., a therapeutically effective amount), are enhanced at a limited dose. Accordingly, some positive effects of the RRx-001, or a pharmaceutically acceptable salt thereof, such as reduction of severity, duration, and onset of adverse side effects are diminished when the RRx-001, or a pharmaceutically acceptable salt thereof (e.g., a therapeutically effective amount), is administered less than a dosage amount, duration of administration, frequency of administration, or combination thereof.

In an exemplary embodiment, the administration of the RRx-001, or a pharmaceutically acceptable salt thereof (e.g., a therapeutically effective amount), is limited to a pretreatment period and is not administered after beginning treatment with a therapeutic agent. In an exemplary embodiment, administration of the RRx-001, or a pharmaceutically acceptable salt thereof (e.g., a therapeutically effective amount), is limited to a portion of a time that a therapeutic agent is administered. For example, the RRx-001, or a pharmaceutically acceptable salt thereof (e.g., a therapeutically effective amount), may be administered for a first portion of a time period that the therapeutic agent is administered.

In an exemplary embodiment, the RRx-001, or a pharmaceutically acceptable salt thereof (e.g., a therapeutically effective amount), is administered concurrently with administration of a therapeutic agent. In an exemplary embodiment, the RRx-001, or a pharmaceutically acceptable salt thereof (e.g., a therapeutically effective amount), is administered for the same time period that a therapeutic agent is administered. For example, the RRx-001, or a pharmaceutically acceptable salt thereof (e.g., a therapeutically effective amount), is administered over a same 6-week period that a therapeutic agent is administered.

### Therapeutic Agent

The therapeutic agent may comprise one or more agents that are administered to a patient to treat a condition. In various embodiments, the therapeutic agent is configured to treat cancer in the patient. A common therapeutic agent comprises radiation therapy and a chemotherapeutic agent. Various treatments comprise one or more chemotherapeutic agents, one or more radiation therapies, or a combination thereof.

Administration of various therapeutic agents may result in adverse side effects. In various embodiments, the adverse side effects include mucositis, dysphagia, dyspepsia, laryngeal inflammation, oral dysesthesia, vomiting, salivary duct inflammation, esophagitis, any gastrointestinal distress, myelosuppression, dermatitis, hair loss or increased creatinine. In various embodiments, the adverse side effects include development of a secondary cancer.

In various embodiments, administration of RRx-001 increases a tolerated dose of one or more therapeutic agents. The term, “maximizing” or “increasing” a tolerated dose, as used herein, may refer to increasing an amount of a dose that a patient can tolerate for each administration. The term, “maximizing” or “increasing” a tolerated dose, as used herein, may refer to increasing a total amount of one or more therapeutic agents that a patient can tolerate over a period of time. The term, “maximizing” or “increasing” a tolerated dose, as used herein, may refer to increasing a frequency of dosing. The term, “maximizing” or “increasing” a tolerated dose, as used herein, may refer to increasing a duration of time of which a therapeutic agent is administered.

In various embodiments, the term, “maximizing” or “increasing” a tolerated dose, may refer to increasing a dosage, frequency of dosing, total dosage over time, and/or combination thereof for a patient that previously had a poorly tolerated dosing amount, frequency of dosing, and/or total dosage amount. The term poorly tolerated, when used herein, refers to an inability of a human patient or animal subject to endure the adverse effects resulting from the use of one or more therapeutic agents. The term poorly tolerated, may refer to one or more adverse effects that cause a human patient or animal subject to discontinue a treatment or decrease a dosing, frequency of dosing, and/or total dosing amount over time of one or more therapeutic agents.

In various embodiments, administration of RRx-001 increases the efficacy of one or more therapeutic agents. An increase in efficacy may refer to an increase of a desired beneficial effect subsequent to administration of the one or more therapeutic agents. An increase in efficacy may refer to an increase of a probability of a desired beneficial effect in a sample of human patients or animal subjects. An increase in efficacy may refer to an increase in a probability of a desired beneficial effect compared to the same treatment of one or more therapeutic agents without RRx-001.

### Radiation Therapy

Radiation therapy is a type of treatment where high energy particles or electromagnetic radiation are directed at a treatment site on the patient. Various forms of radiation may include but are not limited to x-rays, protons, electrons, gamma rays, beta particles, and alpha particle emitters. The term radiation, as used herein, is intended to refer to all forms of radiation treatment. Radiation therapy may be referred to as ionizing radiation.

Radiation will destroy cells to which it is directed if enough radiation is used. Further, cancer and tumor cells are often more susceptible to radiation damage than normal functioning cells. Various forms of radiation treatment comprise directing a beam of radiation at a treatment site from outside the patient. This is often referred to as external beam radiation therapy. IMRT is a type of external beam radiation therapy whereby the radiation is controlled to fit a size of a condition, such as a tumor or cancer. Other forms of radiation treatment work from the inside via implant or injection into the patient.

### Chemotherapeutic Agent

Chemotherapeutic agents are a broad group of medicaments that are administered to treat cancer or tumors in patients. Various chemotherapeutic agents are cytotoxic whereby the chemotherapeutic agent kills cells in the patient. Many chemotherapeutic agents cause adverse effects in patients that are so serious that the patient is forced to delay or discontinue administration of the chemotherapeutic agent. Various chemotherapeutic agents target specific types of cancers or tumors while some have nonspecific use. Examples of chemotherapeutic agents include but are not limited to cyclophosphamide, ifosfamide, chlorambucil, melphalan, temozolomide, carmustine, lomustine, streptozocin, busulfan, procarbazine, cisplatin, carboplatin, oxaliplatin, methotrexate, pemetrexed, cytarabine, 5-Fluorouracil, capecitabine, gemcitabine, 6-mercaptopurine, azathioprine, fludarabine, cladribine, hydroxyurea, irinotecan, topotecan, etoposide, teniposide, vincristine, vinblastine, vinorelbine, docetaxel, paclitaxel, eribulin, ixabepilone, epothilone, bleomycin, actinomycin D, Doxorubicin, daunorubicin, idarubicin, mitomycin, imatinib, dasatinib, nilotinib, erlotinib, gefitinib, afatinib, osimertinib, alectinib, crizotinib, dabrafenib, vemurafenib, encorafenib, trametinib, ibrutinib, acalabrutinib, ruxolitinib, palbociclib, L-asparaginase, bortezomib, carfilzomib, ixazomib, and olaparib.

In various embodiments, a treatment of chemotherapeutic agents comprises at least one of a PARP inhibitor, a tyrosine kinase inhibitor, an EGFR inhibitor, an HDAC inhibitor, a DNA methyltransferase inhibitor, 5-FU, imatinib, hydroxyurea, taxol, an oncolytic virus, a checkpoint inhibitor, and a topoisomerase inhibitor such as irinotecan or doxorubicin.

An exemplary embodiment comprises administering RRx-001 prior to or concurrently with a thiol-based chemoradioprotectant. Examples of a thiol-based chemoradioprotectant include but are not limited to N-acetyl cysteine (NAC), amifostine, sodium thiosulfate (STS), D-methionine, GSH ethyl ester, and GlyNAC (Glycine and N-Acetylcysteine). In various embodiments, RRx-001 prevents or reduces side effects from the thiol-based chemoradioprotectant. The term thiol-based, when used herein, refers to organic and inorganic compounds with at least one sulfur atom in their molecular structure.

In various embodiment, the therapeutic agent comprises at least one of GM-CSF, palifermin, pilocarpine, gabapentin, keratinocyte growth factor (KGF), coconut oil, MucoLox, cetuximab, a checkpoint inhibitor, transforming growth factor beta (TGF-β), an epidermal growth factor, honey, benzydamine, a corticosteroid, rebamipide, an antimicrobial agent, misoprostol, Amifostine, brilacidin, L-glutamine, low-level laser therapy, laser and light therapy, cryotherapy, vitamin E, pentoxyfyilline, GC4419, clonidine, melatonin, a probiotic, actovegin, aloe vera, allopurinol, azithromycin, Black Mulberry Molasses, Glycerin payayor, Qingre Liyan decoction, erythropoietin mouthwash, EC-18, sucralfate, Avasopasem manganese, and lidocaine. In some embodiments, the checkpoint inhibitor is selected from the group consisting of: pembrolizumab and nivolumab. In some embodiments, the antimicrobial agent is selected from the group consisting of: chlorhexidine, polymyxin E, and amphotericin.

### Pharmaceutical Composition

The present disclosure provides compositions or pharmaceutical compositions for treating or preventing normal tissue injury from at least one of radiation and chemotherapy in a subject in need thereof. As a general matter, the pharmaceutical composition contains at least one active agent and a pharmaceutically acceptable carrier. In some embodiments, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders a compound of the present invention orally bioavailable.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

In certain embodiments, it may be desirable to introduce the compositions disclosed herein into the central nervous system by any suitable route, including intraventricular, intrathecal and epidural injection. Intraventricular injection may be facilitated by an intraventricular catheter, for example, attached to a reservoir, such as an Ommaya reservoir.

In some embodiments, the pharmaceutical composition is configured as an inhalable formulation. In some embodiments, the inhalable formulation is configured as a dosage form adapted for pulmonary or nasal administration to the subject. In some embodiments, for example, dosage forms may include those adapted for inhalation such as aerosols and dry powders. In some embodiments, the formulation described herein is suitable for topical delivery to the lung via nose inhalation and/or mouth inhalation. In other embodiments, the compositions disclosed herein may also be administered directly to the lung by inhalation by a number of different devices.

In some embodiments, the inhalable formulation is configured as an aerosol formulation that comprises a propellant. In some embodiments, the propellant can provide energy to deliver molecules of any of the compounds described herein to the lung. Representative propellants are disclosed in U.S. Pat. No. 6,932,962 B1 and U.S. Pat. No. 8,367,734 B1. In some embodiments, the propellant is presented in the aerosol formulation in an amount ranging from 98% to 99% (w/w) relative to the total weight of the aerosol formulation.

In some embodiments, the aerosol formulation further comprises a surfactant, a co-solvent, and/or a pH buffer. The surfactant can give fine dispersions of the compounds described herein in the propellant and can stabilize the mixture of the compounds described herein in the propellant. In some embodiments, the surfactant comprises a fatty acid or a pharmaceutically acceptable salt thereof (e.g., a therapeutically effective amount), a bile salt, a phospholipid, or an alkyl saccharide. In some embodiments, the surfactant is presented in the formulations described herein in an amount of less than 5% (w/w) (e.g., less than 4%, less than 3%, less than 2%, less than 1% by weight) relative to the total weight of the aerosol formulation.

In some embodiments, the co-solvent can help to stabilize the surfactant and improve the dispersion characteristics. In some embodiments, exemplary co-solvents include ethyl alcohol, isopropyl alcohol, propylene glycol, ethylene glycol, propane, butane, isobutane, pentane, dimethyl ether, diethyl ether and the like. In some embodiments, the co-solvent is present in the formulation in an amount ranging from 0.5% to 20% w/w of the total weight of the formulation. In some embodiments, the co-solvent is present in the formulation in an amount ranging from 0.5% to 5% w/w of the total weight of the formulation. In some embodiments, the co-solvent is present in the formulation in an amount ranging from 0.5% to 1.5% (w/w) of the total weight of the formulation. Representative surfactants, co-solvents, and pH buffers are disclosed in U.S. Pat. No. 6,932,962 B1 and U.S. Pat. No. 8,367,734 B1.

In some embodiments, provided herein are combinations containing the aerosol formulation with the propellant and a pressurized bottle or a nebulizer. In some embodiments, the aerosol formulation with the propellant may be packed in pressurized bottles, where a dosage controller may be used with the pressurized bottle to control the amount of drug being administrated in each spray. In some embodiments, the aerosol formulation with the propellant may be packed in pressurized bottles with a dosage controller, where the dosage controller comprises a valve that controls the delivery of a metered amount of the drug.

In some embodiments, the aerosol formulation is propellant-free and comprises the effective amount of the RRx-001 or the pharmaceutical composition and a solvent. In some embodiments, exemplary solvents include water and alcohols, such as ethanol, isopropanol, and glycols, such as propylene glycol, polyethylene glycol, polypropylene glycol, glycol ether, glycerol and polyoxyethylene alcohols. In some embodiments, the solvent is present in the propellant-free aerosol formulation in an amount ranging from about 0.01% to about 90% (w/w), or about 0.01% to about 50% (w/w), or about 0.01% to about 25% (w/w), or about 0.01% to about 10% (w/w), or about 0.01% to about 5% (w/w) relative to the total weight of the aerosol formulation.

In some embodiments, the propellant-free aerosol formulation may further comprise an emulsifying agent. In some embodiments, exemplary emulsifying agents are disclosed in U.S. Pat. No. 9,498,437 B2. In some embodiments, the emulsifying agent is present in the propellant-free aerosol formulations in an amount ranging from about 0.001% to about 50% (w/w), or about 0.001% to about 25% (w/w), or about 0.001% to about 10% (w/w), or about 0.001% to about 2% (w/w), or about 0.001% to about 1% (w/w) relative to the total weight of the aerosol formulation.

In some embodiments, the propellant-free aerosol formulation may further comprise a complexing agent. In some embodiments, exemplary complexing agents include, but are not limited to, ethylenediaminetetraacetic acid (EDTA) or a pharmaceutically acceptable salt thereof (e.g., a therapeutically effective amount), such as the disodium salt, citric acid, nitrilotriacetic acid and the salts thereof, and sodium edetate. Representative complexing agents are disclosed in U.S. Pat. No. 9,498,437 B2. In some embodiments, the complexing agent is present in the propellant-free aerosol formulations in an amount ranging from about 0.001% to about 50% (w/w), or about 0.001% to about 25% (w/w), or about 0.001% to about 10% (w/w), or about 0.001% to about 2% (w/w), or about 0.001% to about 1% (w/w) relative to the total weight of the aerosol formulation.

In some embodiments, the propellant-free aerosol formulation may further comprise a tonicity agent that can adjust the isotonicity of the present formulations. In some embodiments, exemplary tonicity agents include, but are not limited to, sodium chloride, potassium chloride, zinc chloride, calcium chloride or mixtures thereof. Other osmotic adjusting agents may also include, but are not limited to, mannitol, glycerol, and dextrose or mixtures thereof.

Representative tonicity agents are disclosed in U.S. Pat. No. 9,498,437 B2. In some embodiments, the tonicity agent is present in the propellant-free aerosol formulations in an amount ranging from about 0.01% to about 10% (w/w), or about 1% to about 10% (w/w), or about 1% to about 6% (w/w) relative to the total weight of the aerosol formulation. In some embodiments, the aerosol formulation may further comprise the pH buffer.

In some embodiments, provided herein are combinations containing the propellent-free aerosol formulation provided herein and a nebulizer. In some embodiments, the nebulizer can nebulize liquid formulations, including the propellant-free aerosol formulations detailed herein, and produce a nebulized aerosol mist. In some embodiments, the nebulizer may further have an internal baffle, which can selectively remove large droplets from the mist by impaction and allow the droplets to return to the reservoir, so that only fine aerosol droplets are entrained into the lung of the subject by the inhaling air/oxygen. Examples of nebulizers include devices supplied by Sheffield Pharmaceuticals, St. Louis, MO. (Armer et al., U.S. Pat. No. 5,954,047; van der Linden et al., U.S. Pat. No. 5,950,619; van der Linden et al., U.S. Pat. No. 5,970,974) and Batelle Pulmonary Therapeutics, Columbus, OH).

In some embodiments, a Metered Dose Inhaler (“MDI”), which utilizes canisters that contain a suitable low boiling propellant, (e.g., dichlorodifluoromethane, trichlorofluoromethane, dichlorotetrafluoroethane, carbon dioxide or any other suitable gas) may be used to deliver the RRx-001 and/or pharmaceutical compositions thereof directly to the lung. Specifically, the MDI comprises an aerosol container suitable for containing a propellant-based aerosol formulation and/or a metering valve, for example a side valve, which controls the release of the aerosol formulation to the subject. Representative methods and devices to administer the aerosol formulation with the propellant are disclosed in U.S. Pat. No. 9,498,437 B2.

In another embodiment, a Dry Powder Inhaler (“DPI”) device may be used to administer the compositions disclosed herein to the lung. DPI devices typically use a mechanism such as a burst of gas to create a cloud of dry powder inside a container, which may then be inhaled by the patient and are well known in the art. In a particular embodiment, a popular variation is the multiple dose DPI (“MDDPI”) system, which allows for the delivery of more than one therapeutic dose. MDDPI devices are commercially available from a number of pharmaceutical companies e.g., Schering Plough, Madison, NJ). For example, capsules and cartridges of gelatin for use in an inhaler or insufflator may be formulated containing a powder mix of the compositions disclosed herein and a suitable powder base such as lactose or starch for these systems.

In some embodiments, another type of device that may be used to deliver the compositions disclosed herein to the lung is a liquid spray device supplied, for example, by Aradigm Corporation, Hayward, CA. Liquid spray systems use extremely small nozzle holes to aerosolize liquid drug formulations that may then be directly inhaled into the lung.

In some embodiments, a nebulizer is used to deliver the compositions disclosed herein to the lung. Nebulizers create aerosols from liquid drug formulations by using, for example, ultrasonic energy to form fine particles that may be readily inhaled (see e.g., Verschoyle et al., British J. Cancer, 1999, 80, Suppl. 2, 96). Examples of nebulizers include devices supplied by Sheffield Pharmaceuticals, St. Louis, MO. (Armer et al., U.S. Pat. No. 5,954,047; van der Linden et al., U.S. Pat. No. 5,950,619; van der Linden et al., U.S. Pat. No. 5,970,974) and Batelle Pulmonary Therapeutics, Columbus, OH).

In other embodiments, an electrohydrodynamic (“EHD”) aerosol device is used to deliver the compositions disclosed herein to the lung of a patient. EHD aerosol devices use electrical energy to aerosolize liquid drug solutions or suspensions (see e.g., Noakes et al., U.S. Pat. No. 4,765,539). The electrochemical properties of the formulation may be important parameters to optimize when delivering the RRx-001 and/or pharmaceutical composition thereof to the lung with an EHD aerosol device. EHD aerosol devices may more efficiently deliver drugs to the lung than existing pulmonary delivery technologies.

Suitable pharmaceutically acceptable excipients will vary depending upon the particular dosage form chosen. In some embodiments, for example, certain pharmaceutically acceptable excipients may be chosen for their ability to: facilitate the production of aerosol for inhalation, facilitate the production of solution or mist for inhalation, facilitate the production of dry powder for inhalation, or facilitate the production of stable dosage forms.

In some embodiments, the compositions disclosed herein can be delivered via sustained release systems, e.g., oral sustained release systems. In other embodiments, a pump may be used (e.g., Langer, supra, Sefton, 1987, CRC Crit. Ref Biomed. Eng. 14:201; Saudek et al., 1989, N. Engl. J Med. 321:574).

In some embodiments, polymeric materials can be used (e.g., “Medical Applications of Controlled Release,” Langer and Wise (eds.), CRC Press, Boca Raton, Florida (1974); “Controlled Drug Bioavailability,” Drug Product Design and Performance, Smolen and Ball (eds.), Wiley, New York (1984); Ranger et al., 1983, J Macromol. Sci. Rev. Macromol Chem. 23:61; Levy et al., 1985, Science 228:190; During et al., 1989, Ann. Neurol. 25:351; Howard et al., 1989, J. Neurosurg. 71:105).

In other embodiments, polymeric materials are used for oral sustained release delivery. Polymers include, but are not limited to, sodium carboxymethylcellulose, hydroxypropylcellulose, hydroxypropylmethylcellulose and hydroxyethylcellulose (most preferred, hydroxypropyl methylcellulose). Other cellulose ethers have been described (Alderman, Int. J. Pharm. Tech. & Prod. Mfr. 1984, 5(3) 1-9). Factors affecting drug release are well known to the skilled artisan and have been described in the art (Bamba et al., Int. J. Pharm. 1979, 2, 307).

In other embodiments, enteric-coated preparations can be used for oral sustained release administration. Coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In other embodiments, osmotic delivery systems are used for oral sustained release administration (Verma et al., Drug Dev. Ind. Pharm., 2000, 26:695-708). In some embodiments, OROS™ osmotic devices are used for oral sustained release delivery devices (Theeuwes et al., U.S. Pat. No. 3,845,770; Theeuwes et al., U.S. Pat. No. 3,916,899).

In yet other embodiments, a controlled-release system can be placed in proximity of the target of RRx-001 described herein and/or pharmaceutical composition, thus requiring only a fraction of the systemic dose (e.g., Goodson, in “Medical Applications of Controlled Release,” supra, vol. 2, pp. 115-138 (1984)). Other controlled-release systems previously may also be used (Langer, 1990, Science 249:1527-1533).

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In some cases, in order to prolong the effect of a drug, it is desirable to slow the absorption of the drug administered by subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

When the compounds of the present invention are administered as pharmaceuticals to subjects, they can be given per se or as a pharmaceutical composition containing, for example, 0.1 to 99% (more preferably, 10 to 30%) of active ingredient in combination with a pharmaceutically acceptable carrier.

Combination with a Blood Product and/or a Therapeutic Agent

Pharmaceutical compositions for treating or preventing the normal tissue injury from the at least one of the radiation and the chemotherapy in the subject in need thereof are disclosed. The pharmaceutical composition comprises the effective amount of RRx-001, or a pharmaceutically acceptable salt thereof (e.g., a therapeutically effective amount), and at least one pharmaceutically acceptable carrier or excipient. In some embodiments, the pharmaceutical composition for treating or preventing the normal tissue injury from the at least one of the radiation and the chemotherapy in the subject in need thereof comprises (1) an effective amount of RRx-001, or a pharmaceutically acceptable salt thereof and (2) at least one of a blood product and an additional agent.

In some embodiments, the blood product comprises erythrocyte cells. In some embodiments, the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis. In some embodiments, the blood product is a mixture of packed red blood cells. In other embodiments, the blood product is whole blood. In some embodiments, the whole blood is autologous whole blood.

In some embodiments, each additional agent is a therapeutic agent as disclosed herein. In some embodiments, the therapeutic agent is selected from the group consisting of: Dexamethasone, Erbitux (Cetuximab), Avasopasem (GC4419), Pembrolizumab, Nivolumab, Hydrocortisone, Prednisone, Cyclophosphamide, Methotrexate, Paclitaxel, Carboplatin, Etoposide, Gemcitabine, Cisplatin, Oxaliplatin, Chlorambucil, Mechloethamine, Melphalan, Tocilizumab, Brentuximab Vedotin, Doxorubicin, Afatinib, Everolimus, Netupitant, Palonosetron, Imiquimod, and Fluorouracil. In some embodiments, the therapeutic agent is selected from the group consisting of: a PARP inhibitor, a tyrosine kinase inhibitor, a thiol-based chemoradioprotectant agent, an EGFR inhibitor, an HDAC inhibitor, a DNA methyltransferase inhibitor, 5-FU, imatinib, hydroxyurea, taxol, an oncolytic virus, a checkpoint inhibitor, and a topoisomerase inhibitor. Non-limiting examples of the thiol-based chemoradioprotectant agent include NAC, amifostine, STS, D-methionine, GSH ethyl ester, and GlyNAC. Non-limiting examples of the topoisomerase inhibitor comprise irinotecan and doxorubicin. In some examples, the other agent treats oral mucositis and is selected from the group consisting of: GM-CSF, palifermin, pilocarpine, gabapentin, keratinocyte growth factor (KGF), coconut oil, MucoLox, cetuximab, a checkpoint inhibitor, transforming growth factor beta (TGF-β), an epidermal growth factor, honey, benzydamine, a corticosteroid, rebamipide, an antimicrobial agent, misoprostol, Amifostine, brilacidin, L-glutamine, low-level laser therapy, laser and light therapy, cryotherapy, vitamin E, pentoxyfyilline, GC4419, clonidine, melatonin, a probiotic, actovegin, aloe vera, allopurinol, azithromycin, Black Mulberry Molasses, Glycerin payayor, Qingre Liyan decoction, erythropoietin mouthwash, EC-18, sucralfate, Avasopasem manganese, and lidocaine. In some embodiments, the checkpoint inhibitor is selected from the group consisting of: pembrolizumab and nivolumab. In some embodiments, the antimicrobial agent is selected from the group consisting of: chlorhexidine, polymyxin E, and amphotericin.

The present invention can provide methods of attenuating interactions of a first drug (e.g., a first therapeutic agent) and a second drug (e.g., a second therapeutic agent) in a mammal. As described herein, interactions of drugs, or drug-drug interactions, can refer to the changes of the effects of a drug or a pharmaceutical composition on a mammal when the pharmaceutical composition is taken together with a second drug or second pharmaceutical composition. In some embodiments, the interactions can occur when more than two drugs are concurrently in a mammal, regardless of the time between the administrations of the two or more drugs and thereby, and react with each other.

In some embodiments, as described herein, “attenuating interactions” of drugs refers to actions that result in reducing or preventing any types of interactions between two or more drugs or reducing the hypersensitivity, the toxicity, or adverse effects that are caused by the interactions of two or more drugs. In some embodiments, the interactions can include, but are not limited to, synergistic or antagonistic interactions. By way of examples, attenuating interactions of the drugs can be at least any one of the following scenarios: reducing and/or preventing drug-drug physical interactions, reducing and/or preventing drug-drug pharmacokinetic interactions, reducing and/or preventing the hypersensitivity caused by co-existence of the drugs, reducing and/or preventing the toxicity caused by co-existence of drugs, or reducing and/or preventing the antagonistic interactions of drugs.

In some embodiments, the effects of the attenuated interactions can be delayed, decreased, or enhanced absorption of either pharmaceutical composition, and thereby decreases or increases the action of one or more of the additional agent(s) or the pharmaceutical composition. In some embodiments, the attenuated interactions can impact the transport or the distribution of the additional agent(s) or the pharmaceutical compositions.

Accordingly, in certain embodiments, the subject has reduced incidence and/or severity of side effects compared to subjects receiving a direct administration of the same additional agent(s) at the same dose without being mixed with the blood product prior to administration. In certain embodiments, the subject has reduced side effects compared to subjects receiving a direct administration of the same additional agent(s) at the same dose without being mixed with the blood product prior to administration. In certain embodiments, the dose of the additional agent(s) in the pharmaceutical composition is at least about 10% to about 300% more than the dose recommended for a direct administration of the same additional agent(s) without being mixed with the blood product prior to administration. In certain embodiments, the dose of the additional agent(s) in the pharmaceutical composition is at least 1%, at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, or higher, inclusive of all ranges and subranges therebetween, more than the dose recommended for a direct administration of the same additional agent(s) without being mixed with the blood product prior to administration.

In certain embodiments, the additional agent(s) has/have a longer circulating half-life in the subject compared to direct administration of the same additional agent(s) at the same dose without being mixed with the blood product prior to administration. In certain embodiments, the circulating half-life of the additional agent(s) is at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 110%, 110%, 120%, 130%, 140%, 150%, 160%, 170%, 180%, 190%, 200%, 250%, 300%, 350%, 400%, 450%, 500%, 600%, 700%, 800%, 900%, 1000%, or more, longer than the circulating half-life of the same additional agent(s) at the same dose without being mixed with the blood product before administration.

In some embodiments, the subject is administered a pretreatment of the therapeutically effective amount of the RRx-001, or a pharmaceutically acceptable salt thereof, for a frequency during a time period. In some embodiments, the therapeutically effective amount of the RRx-001, or a pharmaceutically acceptable salt thereof, is in a range of about 1 mg to about 50 mg. In some embodiments, the therapeutically effective amount of the RRx-001, or a pharmaceutically acceptable salt thereof, is in the range of about 1 mg to about 25 mg. In some embodiments, the therapeutically effective amount of the RRx-001, or a pharmaceutically acceptable salt thereof, is in the range of about 1 mg to about 20 mg. In some embodiments, the therapeutically effective amount of the RRx-001, or a pharmaceutically acceptable salt thereof, is in the range of about 1 mg to about 15 mg. In some embodiments, the therapeutically effective amount of the RRx-001, or a pharmaceutically acceptable salt thereof, is about 4 mg. In some embodiments, the frequency is between about 4 times per day and about 1 time per week during the time period. In some embodiments, the frequency is between about 2 times per day and about 3 times per week during the time period. In some embodiments, the frequency is two times per week. In some embodiments, the time period is up to six weeks. In some embodiments, the time period is up to four weeks. In some embodiments, the time period is up to two weeks. In some embodiments, the time period is two weeks. In some embodiments, subsequent to administration of the pretreatment of the therapeutically effective amount of the RRx-001, or a pharmaceutically acceptable salt thereof, the subject is administered chemotherapy and IMRT. In some embodiments, the IMRT is about 60 Gy. In some embodiments, the chemotherapy is a therapeutic agent. In some embodiments, the therapeutic agent is cisplatin. In some embodiments, the chemotherapy and the IMRT are administered once per week, once every two weeks, once every three weeks, once every four weeks, once every five weeks, etc. In some embodiments, an amount of the cisplatin is in a range of 5 mg/m² to 300 mg/m². In some embodiments, the amount of the cisplatin is in the range of 10 mg/m² to 200 mg/m². In some embodiments, the amount of the cisplatin is in the range of 20 mg/m² to 100 mg/m². In some embodiments, about 40 mg/m² of the cisplatin is administered each week. In some embodiments, about 100 mg/m² of the cisplatin is administered once every three weeks. In some embodiments, the therapeutically effective amount of the RRx-001, or a pharmaceutically acceptable salt thereof, is not administered during the treatment period in which the subject is treated with the chemotherapy and/or the IMRT (e.g., it is used only as a pretreatment and not as a cotreatment).

In some embodiments, the subject is administered the pretreatment of the therapeutically effective amount of the RRx-001, or a pharmaceutically acceptable salt thereof, in an amount of 4 mg for a frequency of twice per week during a time period of two weeks, followed by 40 mg/m² each week or 100 mg/m² once every 3 weeks cisplatin+60 Gy of IMRT. In this embodiment, the therapeutically effective amount of the RRx-001, or a pharmaceutically acceptable salt thereof, is not administered during the treatment period in which the subject is treated with the chemotherapy and/or the IMRT (e.g., it is used only as the pretreatment and not as the cotreatment).

In some embodiments where the therapeutically effective amount of the RRx-001, or a pharmaceutically acceptable salt thereof, is administered to the subject as the pretreatment, a low dose of RRx-001, or a pharmaceutically acceptable salt thereof, lowers a duration of SOM as compared to no dose or a high dose. Further, in some embodiments where the therapeutically effective amount of the RRx-001, or a pharmaceutically acceptable salt thereof, is administered to the subject as the pretreatment, the low dose of RRx-001, or a pharmaceutically acceptable salt thereof, the subject has no incidence of Grade 4 OM through 60 Gy. Subjects who receive the low dose of RRx-001, or a pharmaceutically acceptable salt thereof, concurrently with the cisplatin are able to tolerate higher doses of the cisplatin compared to the SOC control. In some embodiments where the therapeutically effective amount of the RRx-001, or a pharmaceutically acceptable salt thereof, is administered to the subject as the pretreatment, the low dose of RRx-001, or a pharmaceutically acceptable salt thereof, decreases the occurrence of dyspepsia, dyspnea, and/or laryngeal inflammation.

In some embodiments, the subject is administered the pretreatment of the therapeutically effective amount of the RRx-001, or a pharmaceutically acceptable salt thereof, for the frequency during the time period. In some embodiments, the therapeutically effective amount of the RRx-001, or a pharmaceutically acceptable salt thereof, is in a range of about 1 mg to about 50 mg. In some embodiments, the therapeutically effective amount of the RRx-001, or a pharmaceutically acceptable salt thereof, is in the range of about 1 mg to about 25 mg. In some embodiments, the therapeutically effective amount of the RRx-001, or a pharmaceutically acceptable salt thereof, is in the range of about 1 mg to about 20 mg. In some embodiments, the therapeutically effective amount of the RRx-001, or a pharmaceutically acceptable salt thereof, is in the range of about 1 mg to about 15 mg. In some embodiments, the therapeutically effective amount of the RRx-001, or a pharmaceutically acceptable salt thereof, is about 4 mg. In some embodiments, the frequency is between about 4 times per day and about 1 time per week during the time period. In some embodiments, the frequency is between about 2 times per day and about 3 times per week during the time period. In some embodiments, the frequency is two times per week. In some embodiments, the time period is up to six weeks. In some embodiments, the time period is up to four weeks. In some embodiments, the time period is up to two weeks. In some embodiments, the time period is two weeks. In some embodiments, the pretreatment is followed by co-treatment with the therapeutically effective amount of the RRx-001, or a pharmaceutically acceptable salt thereof, for another frequency during another time period. In some embodiments, the therapeutically effective amount of the co-treatment of the RRx-001, or a pharmaceutically acceptable salt thereof, is in a range of about 1 mg to about 50 mg. In some embodiments, the therapeutically effective amount of the co-treatment of the RRx-001, or a pharmaceutically acceptable salt thereof, is in the range of about 1 mg to about 25 mg. In some embodiments, the therapeutically effective amount of the co-treatment of the RRx-001, or a pharmaceutically acceptable salt thereof, is in the range of about 1 mg to about 20 mg. In some embodiments, the therapeutically effective amount of the co-treatment of the RRx-001, or a pharmaceutically acceptable salt thereof, is in the range of about 1 mg to about 15 mg. In some embodiments, the therapeutically effective amount of the co-treatment of the RRx-001, or a pharmaceutically acceptable salt thereof, is about 4 mg. In some embodiments, the other frequency is once per week, twice per week, three times per week, four times per week, etc. and the other time period is one day, one week, two weeks, three weeks, one month, etc. In some embodiments, the other frequency is twice on week 2 and once on week 5. In some embodiments, subsequent administration of the pretreatment and the co-treatment of the therapeutically effective amount of the RRx-001, or a pharmaceutically acceptable salt thereof, the subject is administered chemotherapy and IMRT. In some embodiments, he IMRT is about 60 Gy. In some embodiments, the chemotherapy is a therapeutic agent. In some embodiments, the therapeutic agent is a platinum-based agent. In some embodiments, the therapeutic agent is cisplatin. In some embodiments, the chemotherapy and the IMRT are administered once per week, once every two weeks, once every three weeks, once every four weeks, once every five weeks, etc. In some embodiments, an amount of the platinum-based agent (e.g., cisplatin) is in a range of 5 mg/m² to 300 mg/m². In some embodiments, the amount of the platinum-based agent (e.g., cisplatin) is in the range of 10 mg/m² to 200 mg/m². In some embodiments, the amount of the platinum-based agent (e.g., cisplatin) is in the range of 20 mg/m² to 100 mg/m². In some embodiments, about 40 mg/m² of the platinum-based agent (e.g., cisplatin) is administered each week. In some embodiments, about 100 mg/m² of the platinum-based agent (e.g., cisplatin) is administered once every three weeks.

In some embodiments, the subject is administered a pretreatment of the therapeutically effective amount of the RRx-001, or a pharmaceutically acceptable salt thereof, in an amount of 4 mg for a frequency of twice per week during a time period of two weeks, followed by co-treatment with 4 mg of the therapeutically effective amount of the RRx-001, or a pharmaceutically acceptable salt thereof, six times per week and 40 mg/m² each week or 100 mg/m² once every 3 weeks cisplatin+60 Gy of the IMRT.

In some embodiments, the subject is administered the pretreatment of the therapeutically effective amount of the RRx-001, or a pharmaceutically acceptable salt thereof, for the frequency during the time period. In some embodiments, the therapeutically effective amount of the RRx-001, or a pharmaceutically acceptable salt thereof, is in a range of about 1 mg to about 50 mg. In some embodiments, the therapeutically effective amount of the RRx-001, or a pharmaceutically acceptable salt thereof, is in the range of about 1 mg to about 25 mg. In some embodiments, the therapeutically effective amount of the RRx-001, or a pharmaceutically acceptable salt thereof, is in the range of about 1 mg to about 20 mg. In some embodiments, the therapeutically effective amount of the RRx-001, or a pharmaceutically acceptable salt thereof, is in the range of about 1 mg to about 15 mg. In some embodiments, the therapeutically effective amount of the RRx-001, or a pharmaceutically acceptable salt thereof, is about 4 mg. In some embodiments, the frequency is between about 4 times per day and about 1 time per week during the time period. In some embodiments, the frequency is between about 2 times per day and about 3 times per week during the time period. In some embodiments, the frequency is two times per week. In some embodiments, the time period is up to six weeks. In some embodiments, the time period is up to four weeks. In some embodiments, the time period is up to two weeks. In some embodiments, the time period is two weeks. In some embodiments, the pretreatment is followed by co-treatment with the therapeutically effective amount of the RRx-001, or a pharmaceutically acceptable salt thereof, for another frequency during another time period. In some embodiments, the therapeutically effective amount of the co-treatment of the RRx-001, or a pharmaceutically acceptable salt thereof, is in a range of about 1 mg to about 50 mg. In some embodiments, the therapeutically effective amount of the co-treatment of the RRx-001, or a pharmaceutically acceptable salt thereof, is in the range of about 1 mg to about 25 mg. In some embodiments, the therapeutically effective amount of the co-treatment of the RRx-001, or a pharmaceutically acceptable salt thereof, is in the range of about 1 mg to about 20 mg. In some embodiments, the therapeutically effective amount of the co-treatment of the RRx-001, or a pharmaceutically acceptable salt thereof, is in the range of about 1 mg to about 15 mg. In some embodiments, the therapeutically effective amount of the co-treatment of the RRx-001, or a pharmaceutically acceptable salt thereof, is about 4 mg. In some embodiments, the other frequency is once per week, twice per week, three times per week, four times per week, five times per week, six times per week, seven times per week, eight times per week, etc. and the other time period is one day, one week, two weeks, three weeks, four weeks, five weeks, six weeks, seven weeks, eight weeks, nine weeks, ten weeks, etc. In some embodiments, the other frequency is six times per week. In some embodiments, subsequent administration of the pretreatment and the co-treatment of the therapeutically effective amount of the RRx-001, or a pharmaceutically acceptable salt thereof, the subject is administered chemotherapy and IMRT. The IMRT is about 60 Gy. In some embodiments, the chemotherapy is a therapeutic agent. In some embodiments, the therapeutic agent is cisplatin. In some embodiments, the chemotherapy and the IMRT are administered once per week, once every two weeks, once every three weeks, once every four weeks, once every five weeks, etc. In some embodiments, an amount of the cisplatin is in a range of 5 mg/m² to 300 mg/m². In some embodiments, the amount of the cisplatin is in the range of 10 mg/m² to 200 mg/m². In some embodiments, the amount of the cisplatin is in the range of 20 mg/m² to 100 mg/m². In some embodiments, about 40 mg/m² of the cisplatin is administered each week. In some embodiments, about 100 mg/m² of the cisplatin is administered once every three weeks.

In some embodiments, the subject has a pathologically diagnosed stage III to IVB locally advanced (nonmetastatic) squamous cell carcinoma of the oral cavity or oropharynx. In some embodiments, the subject is first administered a pre-medication dose of a corticosteroid. In some embodiments, the corticosteroid is dexamethasone. In some embodiments, an amount of the dexamethasone is between about 1 mg to about 50 mg. In some embodiments, the amount of the dexamethasone is between about 5 mg to about 45 mg. In some embodiments, the amount of the dexamethasone is about 10 mg. In some embodiments, the subject is then administered a pretreatment of the therapeutically effective amount of the RRx-001, or a pharmaceutically acceptable salt thereof, for a frequency during a time period. In some embodiments, the therapeutically effective amount of the RRx-001, or a pharmaceutically acceptable salt thereof, is in a range of about 1 mg to about 50 mg. In some embodiments, the therapeutically effective amount of the RRx-001, or a pharmaceutically acceptable salt thereof, is in the range of about 1 mg to about 25 mg. In some embodiments, the therapeutically effective amount of the RRx-001, or a pharmaceutically acceptable salt thereof, is in the range of about 1 mg to about 20 mg. In some embodiments, the therapeutically effective amount of the RRx-001, or a pharmaceutically acceptable salt thereof, is in the range of about 1 mg to about 15 mg. In some embodiments, the therapeutically effective amount of the RRx-001, or a pharmaceutically acceptable salt thereof, is about 4 mg. In some embodiments, the frequency is between about 4 times per day and about 1 time per week during the time period. In some embodiments, the frequency is between about 2 times per day and about 3 times per week during the time period. In some embodiments, the frequency is two times per week. In some embodiments, the time period is up to six weeks. In some embodiments, the time period is up to four weeks. In some embodiments, the time period is up to two weeks. In some embodiments, the time period is two weeks. In some embodiments, subsequent administration of the pretreatment of the therapeutically effective amount of the RRx-001, or a pharmaceutically acceptable salt thereof, the subject is administered chemotherapy and IMRT. In some embodiments, the chemotherapy is a therapeutic agent. In some embodiments, the therapeutic agent is cisplatin. In some embodiments, the chemotherapy and the IMRT are administered once per week, once every two weeks, once every three weeks, once every four weeks, once every five weeks, etc. In some embodiments, the IMRT is 2.0 Gy to 2.2 Gy, for a total dose of 60 Gy to 72 Gy. In some embodiments, an amount of the cisplatin is in a range of 5 mg/m² to 300 mg/m². In some embodiments, the amount of the cisplatin is in the range of 10 mg/m² to 200 mg/m². In some embodiments, the amount of the cisplatin is in the range of 20 mg/m² to 100 mg/m². In some embodiments, about 40 mg/m² of the cisplatin is administered each week. In some embodiments, about 100 mg/m² of the cisplatin is administered once every three weeks.

In some embodiments, the subject has a pathologically diagnosed stage III to IVB locally advanced (nonmetastatic) squamous cell carcinoma of the oral cavity or oropharynx. In some embodiments, the subject is first administered a pre-medication dose of 10 mg of dexamethasone. In some embodiments, the subject is then administered a pretreatment of 4 mg of the therapeutically effective amount of the RRx-001, or a pharmaceutically acceptable salt thereof, for a frequency of two times per week during a time period of two weeks. In some embodiments, subsequent administration of the pretreatment of the therapeutically effective amount of the RRx-001, or a pharmaceutically acceptable salt thereof, the subject is administered 40 mg/m² each week or 100 mg/m² once every 3 weeks cisplatin+60 Gy to 72 Gy of IMRT. In this embodiment, the therapeutically effective amount of the RRx-001, or a pharmaceutically acceptable salt thereof, is not administered during the treatment period in which the subject is treated with the chemotherapy and/or the IMRT (e.g., it is used only as the pretreatment and not as the cotreatment).

In some embodiments, administering the therapeutically effective amount of the RRx-001, or a pharmaceutically acceptable salt thereof, as the pretreatment reduces the duration of SOM between the first to the last radiation visit. In some embodiments, administering the therapeutically effective amount of the RRx-001, or a pharmaceutically acceptable salt thereof, as the pretreatment delays the onset of SOM in the subject. In some embodiments, administering the therapeutically effective amount of the RRx-001, or a pharmaceutically acceptable salt thereof, as the pretreatment delays the onset of SOM in the subject by at least one day, at least two days, at least three days, at least four days, at least five days, at least six days, at least seven days, at least two weeks, at least three weeks, at least four weeks, etc. In some embodiments, administering the therapeutically effective amount of the RRx-001, or a pharmaceutically acceptable salt thereof, as the pretreatment results in less soreness associated with OM for the patient.

In some embodiments, administering the therapeutically effective amount of the RRx-001, or a pharmaceutically acceptable salt thereof, as the pretreatment prior to the start of CRT results in a shorter duration of SOM, less incidences of the severe form of OM, and/or a lower incidence of one or more symptoms of OM. In some embodiments, administering the therapeutically effective amount of the RRx-001, or a pharmaceutically acceptable salt thereof, as the pretreatment results in a lower incidence of dry mouth, dysphagia, salivary duct inflammation, radiation-induced skin injury, oral pain, weight loss, anemia, constipation, oral dysesthesia, vomiting, and/or neutrophil count decrease. In some embodiments, administering the therapeutically effective amount of the RRx-001, or a pharmaceutically acceptable salt thereof, as the pretreatment results in a decrease in other toxicities associated with mucositis.

In some embodiments, the subject has a pathologically diagnosed stage III to IVB locally advanced (nonmetastatic) squamous cell carcinoma of the oral cavity or oropharynx. In some embodiments, the subject is first administered a pre-medication dose of a corticosteroid. In some embodiments, the corticosteroid is dexamethasone. In some embodiments, an amount of the dexamethasone is between about 1 mg to about 50 mg. In some embodiments, the amount of the dexamethasone is between about 5 mg to about 45 mg. In some embodiments, the amount of the dexamethasone is about 10 mg. In some embodiments, the subject is then administered a pretreatment of the therapeutically effective amount of the RRx-001, or a pharmaceutically acceptable salt thereof, for a frequency during a time period. In some embodiments, the therapeutically effective amount of the RRx-001, or a pharmaceutically acceptable salt thereof, is in a range of about 1 mg to about 50 mg. In some embodiments, the therapeutically effective amount of the RRx-001, or a pharmaceutically acceptable salt thereof, is in the range of about 1 mg to about 25 mg. In some embodiments, the therapeutically effective amount of the RRx-001, or a pharmaceutically acceptable salt thereof, is in the range of about 1 mg to about 20 mg. In some embodiments, the therapeutically effective amount of the RRx-001, or a pharmaceutically acceptable salt thereof, is in the range of about 1 mg to about 15 mg. In some embodiments, the therapeutically effective amount of the RRx-001, or a pharmaceutically acceptable salt thereof, is about 4 mg. In some embodiments, the frequency is between about 4 times per day and about 1 time per week during the time period. In some embodiments, the frequency is between about 2 times per day and about 3 times per week during the time period. In some embodiments, the frequency is two times per week. In some embodiments, the time period is up to six weeks. In some embodiments, the time period is up to four weeks. In some embodiments, the time period is up to two weeks. In some embodiments, the time period is two weeks. In some embodiments, subsequent administration of the pretreatment of the therapeutically effective amount of the RRx-001, or a pharmaceutically acceptable salt thereof, the subject is administered chemotherapy and IMRT. In some embodiments, the chemotherapy is a therapeutic agent. In some embodiments, the therapeutic agent is cisplatin. In some embodiments, the chemotherapy and the IMRT are administered once per week, once every two weeks, once every three weeks, once every four weeks, once every five weeks, etc. In some embodiments, the IMRT is 2.0 Gy to 2.2 Gy, for a total dose of 60 Gy to 72 Gy. In some embodiments, an amount of the cisplatin is in a range of 5 mg/m² to 300 mg/m². In some embodiments, the amount of the cisplatin is in the range of 10 mg/m² to 200 mg/m². In some embodiments, the amount of the cisplatin is in the range of 20 mg/m² to 100 mg/m². In some embodiments, about 40 mg/m² of the cisplatin is administered each week. In some embodiments, about 100 mg/m² of the cisplatin is administered once every three weeks. In some embodiments, the subject is administered additional doses of the therapeutically effective amount of the RRx-001, or a pharmaceutically acceptable salt thereof, during the IMRT and the chemotherapy. In some embodiments, a quantity of the additional doses is two, three, four, five, six, etc. In some embodiments, the therapeutically effective amount of the RRx-001, or a pharmaceutically acceptable salt thereof, of each additional dose is in the range of about 1 mg to about 15 mg. In some embodiments, the therapeutically effective amount of the RRx-001, or a pharmaceutically acceptable salt thereof, of each additional dose is about 4 mg. In some embodiments, at least one of the additional doses is administered during week 1, week 2, week 3, week 4, week 5, week 6, etc. of the IMRT and the chemotherapy. In some embodiments, one of the additional doses is administered during week 2 and one of the additional doses is administered during week 5 of the IMRT and the chemotherapy. In some embodiments, administering the therapeutically effective amount of the RRx-001, or a pharmaceutically acceptable salt thereof, as the pretreatment results in a lower incidence of dry mouth, dysphagia, salivary duct inflammation, radiation-induced skin injury, oral pain, weight loss, anemia, constipation, oral dysesthesia, vomiting, and neutrophil count decrease.

In some embodiments, the subject has a pathologically diagnosed stage III to IVB locally advanced (nonmetastatic) squamous cell carcinoma of the oral cavity or oropharynx. In some embodiments, the subject is first administered the pre-medication dose of 10 mg of dexamethasone. In some embodiments, the subject is then administered the pretreatment of 4 mg of the therapeutically effective amount of the RRx-001, or a pharmaceutically acceptable salt thereof, for the frequency of two times per week during the time period of two weeks. In some embodiments, subsequent administration of the pretreatment of the therapeutically effective amount of the RRx-001, or a pharmaceutically acceptable salt thereof, the subject is administered 40 mg/m² each week or 100 mg/m² once every 3 weeks cisplatin+60 Gy to 72 Gy of IMRT. In some embodiments, the subject receives two additional 4 mg doses of the therapeutically effective amount of the RRx-001, or a pharmaceutically acceptable salt thereof, during weeks 2 and 5 of the CRT regimen.

In some embodiments, the subject has a pathologically diagnosed stage III to IVB locally advanced (nonmetastatic) squamous cell carcinoma of the oral cavity or oropharynx. In some embodiments, the subject is first administered a pre-medication dose of a corticosteroid. In some embodiments, the corticosteroid is dexamethasone. In some embodiments, an amount of the dexamethasone is between about 1 mg to about 50 mg. In some embodiments, the amount of the dexamethasone is between about 5 mg to about 45 mg. In some embodiments, the amount of the dexamethasone is about 10 mg. In some embodiments, the subject is then administered the therapeutically effective amount of the RRx-001, or a pharmaceutically acceptable salt thereof, for a frequency during a time period. In some embodiments, the therapeutically effective amount of the RRx-001, or a pharmaceutically acceptable salt thereof, is in a range of about 1 mg to about 50 mg. In some embodiments, the therapeutically effective amount of the RRx-001, or a pharmaceutically acceptable salt thereof, is in the range of about 1 mg to about 25 mg. In some embodiments, the therapeutically effective amount of the RRx-001, or a pharmaceutically acceptable salt thereof, is in the range of about 1 mg to about 20 mg. In some embodiments, the therapeutically effective amount of the RRx-001, or a pharmaceutically acceptable salt thereof, is in the range of about 1 mg to about 15 mg. In some embodiments, the therapeutically effective amount of the RRx-001, or a pharmaceutically acceptable salt thereof, is about 4 mg. In some embodiments, the frequency is between about 4 times per day and about 1 time per week during the time period. In some embodiments, the frequency is between about 2 times per day and about 3 times per week during the time period. In some embodiments, the frequency is once per week and the time period is up to six weeks. In some embodiments, the frequency is once per week and the time period is up to six weeks while the patient is administered chemotherapy and IMRT. In some embodiments, the chemotherapy is a therapeutic agent. In some embodiments, the therapeutic agent is cisplatin. In some embodiments, the chemotherapy and the IMRT are administered once per week, once every two weeks, once every three weeks, once every four weeks, once every five weeks, etc. In some embodiments, the IMRT is 2.0 Gy to 2.2 Gy, for a total dose of 60 Gy to 72 Gy. In some embodiments, an amount of the cisplatin is in a range of 5 mg/m² to 300 mg/m². In some embodiments, the amount of the cisplatin is in the range of 10 mg/m² to 200 mg/m². In some embodiments, the amount of the cisplatin is in the range of 20 mg/m² to 100 mg/m². In some embodiments, about 40 mg/m² of the cisplatin is administered each week. In some embodiments, about 100 mg/m² of the cisplatin is administered once every three weeks.

In some embodiments, the subject has a pathologically diagnosed stage III to IVB locally advanced (nonmetastatic) squamous cell carcinoma of the oral cavity or oropharynx. In some embodiments, the subject is first administered the pre-medication dose of 10 mg of dexamethasone. In some embodiments, the subject is then administered the pretreatment of 4 mg of the therapeutically effective amount of the RRx-001, or a pharmaceutically acceptable salt thereof, for a frequency of two times per week during a time period of two weeks. In some embodiments, subsequent administration of the pretreatment of the therapeutically effective amount of the RRx-001, or a pharmaceutically acceptable salt thereof, the subject is administered 40 mg/m² each week or 100 mg/m² once every 3 weeks cisplatin+60 Gy to 72 Gy of IMRT. In some embodiments, the subject receives weekly 4 mg doses of the therapeutically effective amount of the RRx-001, or a pharmaceutically acceptable salt thereof, after the dexamethasone pre-dosing and during the first 6 weeks of CRT.

In some embodiments, the subject has a pathologically diagnosed locally advanced SCC of the oral cavity or oropharynx. In some embodiments, the subject is first administered a pre-medication dose of a corticosteroid. In some embodiments, the corticosteroid is dexamethasone. In some embodiments, an amount of the dexamethasone is between about 1 mg to about 50 mg. In some embodiments, the amount of the dexamethasone is between about 5 mg to about 45 mg. In some embodiments, the amount of the dexamethasone is about 10 mg. In some embodiments, the subject is then administered a pretreatment of the therapeutically effective amount of the RRx-001, or a pharmaceutically acceptable salt thereof, for a frequency during a time period. In some embodiments, the therapeutically effective amount of the RRx-001, or a pharmaceutically acceptable salt thereof, is in a range of about 1 mg to about 50 mg. In some embodiments, the therapeutically effective amount of the RRx-001, or a pharmaceutically acceptable salt thereof, is in the range of about 1 mg to about 25 mg. In some embodiments, the therapeutically effective amount of the RRx-001, or a pharmaceutically acceptable salt thereof, is in the range of about 1 mg to about 20 mg. In some embodiments, the therapeutically effective amount of the RRx-001, or a pharmaceutically acceptable salt thereof, is in the range of about 1 mg to about 15 mg. In some embodiments, the therapeutically effective amount of the RRx-001, or a pharmaceutically acceptable salt thereof, is about 8 mg. In some embodiments, the frequency is between about 4 times per day and about 1 time per week during the time period. In some embodiments, the frequency is between about 2 times per day and about 3 times per week during the time period. In some embodiments, the frequency is two times per week. In some embodiments, the time period is up to six weeks. In some embodiments, the time period is up to four weeks. In some embodiments, the time period is up to two weeks. In some embodiments, the time period is two weeks. In some embodiments, subsequent administration of the pretreatment of the therapeutically effective amount of the RRx-001, or a pharmaceutically acceptable salt thereof, the subject is administered chemotherapy and IMRT. In some embodiments, the chemotherapy is a therapeutic agent. In some embodiments, the therapeutic agent is cisplatin. In some embodiments, the chemotherapy and the IMRT are administered once per week, once every two weeks, once every three weeks, once every four weeks, once every five weeks, once every six weeks, once every seven weeks, once every eight weeks, etc. In some embodiments, the IMRT is 2.0 Gy to 2.2 Gy, for a total dose of 60 Gy to 72 Gy. In some embodiments, an amount of the cisplatin is in a range of 5 mg/m² to 300 mg/m². In some embodiments, the amount of the cisplatin is in the range of 10 mg/m² to 200 mg/m². In some embodiments, the amount of the cisplatin is in the range of 20 mg/m² to 100 mg/m². In some embodiments, about 40 mg/m² of the cisplatin is administered each week for a time period. In some embodiments, about 100 mg/m² of the cisplatin is administered once every three weeks.

In some embodiments, the subject has a pathologically confirmed diagnosis of SCC of the oral cavity or oropharynx. In some embodiments, the subject is administered the premedication dose of 10 mg of dexamethasone or another steroid prior to administration of the pretreatment of 8 mg of the therapeutically effective amount of the RRx-001, or a pharmaceutically acceptable salt thereof, for a frequency of two times per week during a time period of two weeks. In some embodiments, subsequent administration of the pretreatment of the therapeutically effective amount of the RRx-001, or a pharmaceutically acceptable salt thereof, the subject is administered a total dose of 60 Gy to 72 Gy IMRT and 100 mg/m² of cisplatin every 3 weeks or 40 mg/m² of cisplatin weekly for seven weeks. In some embodiments, the therapeutically effective amount of the RRx-001, or a pharmaceutically acceptable salt thereof, is not administered during the treatment period in which the subject is treated with the chemotherapy and IMRT (e.g., it is used only as the pretreatment and not as the cotreatment).

In some embodiments, the subject has a pathologically diagnosed locally advanced SCC of the oral cavity or oropharynx. In some embodiments, the subject is first administered a pre-medication dose of a corticosteroid. In some embodiments, the corticosteroid is dexamethasone. In some embodiments, an amount of the dexamethasone is between about 1 mg to about 50 mg. In some embodiments, the amount of the dexamethasone is between about 5 mg to about 45 mg. In some embodiments, the amount of the dexamethasone is about 10 mg. In some embodiments, the subject is then administered a pretreatment of the therapeutically effective amount of the RRx-001, or a pharmaceutically acceptable salt thereof, for a frequency during a time period. In some embodiments, the therapeutically effective amount of the RRx-001, or a pharmaceutically acceptable salt thereof, is in a range of about 1 mg to about 50 mg. In some embodiments, the therapeutically effective amount of the RRx-001, or a pharmaceutically acceptable salt thereof, is in the range of about 1 mg to about 25 mg. In some embodiments, the therapeutically effective amount of the RRx-001, or a pharmaceutically acceptable salt thereof, is in the range of about 1 mg to about 20 mg. In some embodiments, the therapeutically effective amount of the RRx-001, or a pharmaceutically acceptable salt thereof, is in the range of about 1 mg to about 15 mg. In some embodiments, the therapeutically effective amount of the RRx-001, or a pharmaceutically acceptable salt thereof, is about 4 mg. In some embodiments, the frequency is between about 4 times per day and about 1 time per week during the time period. In some embodiments, the frequency is between about 2 times per day and about 3 times per week during the time period. In some embodiments, the frequency is two times per week. In some embodiments, the time period is up to six weeks. In some embodiments, the time period is up to four weeks. In some embodiments, the time period is up to two weeks. In some embodiments, the time period is two weeks. In some embodiments, subsequent administration of the pretreatment of the therapeutically effective amount of the RRx-001, or a pharmaceutically acceptable salt thereof, the subject is administered chemotherapy and IMRT. In some embodiments, the chemotherapy is a therapeutic agent. In some embodiments, the therapeutic agent is cisplatin. In some embodiments, the chemotherapy and the IMRT are administered once per week, once every two weeks, once every three weeks, once every four weeks, once every five weeks, once every six weeks, once every seven weeks, once every eight weeks, once every nine weeks, etc. In some embodiments, the IMRT is 2.0 Gy to 2.2 Gy, for a total dose of 60 Gy to 72 Gy. In some embodiments, an amount of the cisplatin is in a range of 5 mg/m² to 300 mg/m². In some embodiments, the amount of the cisplatin is in the range of 10 mg/m² to 200 mg/m². In some embodiments, the amount of the cisplatin is in the range of 20 mg/m² to 100 mg/m². In some embodiments, about 40 mg/m² of the cisplatin is administered each week for a time period. In some embodiments, about 100 mg/m² of the cisplatin is administered once every three weeks.

In some embodiments, the subject has a pathologically confirmed diagnosis of SCC of the oral cavity or oropharynx. In some embodiments, the subject is administered the premedication dose of 10 mg of dexamethasone or another steroid prior to administration of the pretreatment of 4 mg of the therapeutically effective amount of the RRx-001, or a pharmaceutically acceptable salt thereof, for a frequency of two times per week during a time period of two weeks. In some embodiments, subsequent administration of the pretreatment of the therapeutically effective amount of the RRx-001, or a pharmaceutically acceptable salt thereof, the subject is administered a total dose of 60 Gy to 72 Gy IMRT and 100 mg/m² of cisplatin every 3 weeks or 40 mg/m² of cisplatin weekly for seven weeks. In some embodiments, the therapeutically effective amount of the RRx-001, or a pharmaceutically acceptable salt thereof, is not administered during the treatment period in which the subject is treated with the chemotherapy and IMRT (e.g., it is used only as the pretreatment and not as the cotreatment).

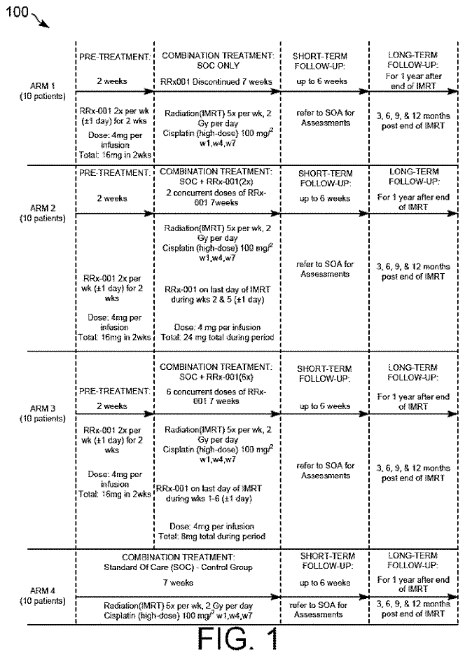

Referring to FIG. 1, FIG. 1 is a table 100 showing a trial in which RRx-001 was tested in various groups of patients that were being treated for cancer with a combination of chemotherapy and IMRT. The various groups were divided into “Arms” based on a treatment:
Arm 1. pretreatment with 4 mg RRx-001 2 times per week for 2 weeks followed by 40 mg/m² each week or 100 mg/m² once every 3 weeks cisplatin+IMRT.
Arm 2. pretreatment with 4 mg RRx-001 2 times per week for 2 weeks followed by cotreatment with 4 mg RRx-001 2 times once on week 2 and once on week 5+40 mg/m² each week or 100 mg/m² once every 3 weeks cisplatin+IMRT.
Arm 3. pretreatment with 4 mg RRx-001 2 times per week for 2 weeks followed by cotreatment with 4 mg RRx-001 6 times per week+40 mg/m² each week or 100 mg/m² once every 3 weeks cisplatin+IMRT.
Arm 4. standard of care 40 mg/m² each week or 100 mg/m² once every 3 weeks cisplatin+IMRT without RRx-001.

Accordingly, patients in Arm 1 of the study were administered a pretreatment of RRx-001 over a 2-week period. No RRx-001 was administered to the patients following the pretreatment period. Following the 2-week pretreatment period, the therapeutic agent comprising cisplatin and IMRT was administered to the patients over a 6-week period.

Patients in Arm 2 of the study were likewise administered a pretreatment of RRx-001 over a 2-week period. But RRx-001 was further administered at a low dose concurrently with cisplatin and IMRT over the 6-week period following the pretreatment period. Patients in Arm 3 of the study had a similar treatment to Arm 2 with the difference in that patients in Arm 3 were administered a higher amount of RRx-001 over the 6-week period following the pretreatment period.

Arm 4 of the study was the control or standard of care (SOC). No RRx-001 was administered to the patients in Arm 4 of the study and there was no pretreatment period. All patients received the same amounts of cisplatin and IMRT for Arm 1, Arm2, Arm 3, and Arm 4 of the study. Patients in the study had the option to stop or refuse treatment at any time and not all patients finished the entire treatment.

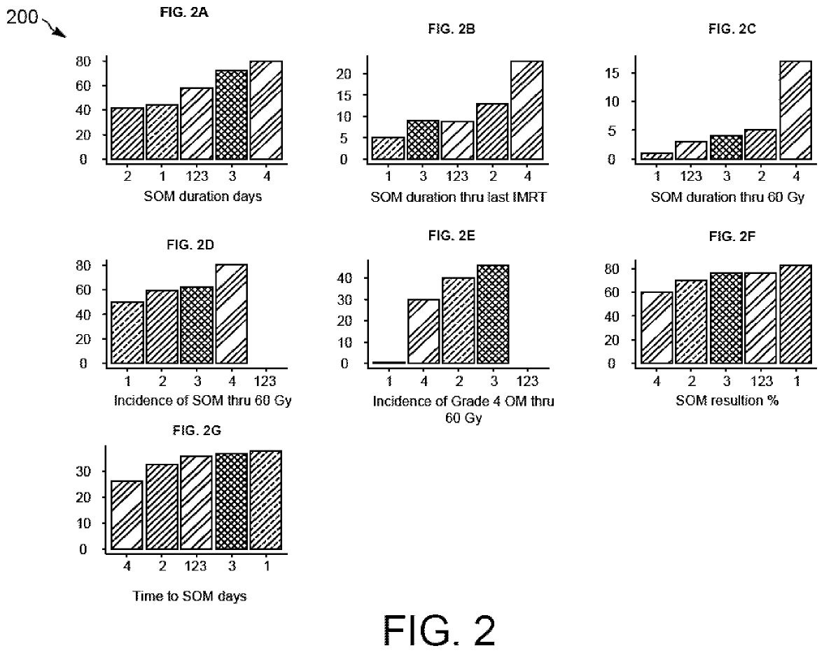

Referring to FIG. 2, FIG. 2 shows 7 bar graphs 200. FIG. 2A shows the duration of severe oral mucositis (SOM) in patients for Arms 1, 2, 3, and 4 as a number of days from the onset of SOM to the day when SOM was resolved. FIG. 2B shows a duration of SOM in patients following a last treatment of IMRT for Arms 1, 2, 3, and 4. FIG. 2C shows a duration of SOM following treatment of 60 gray (Gy) of IMRT for Arms 1,2,3, and 4. FIG. 2D shows a percentage of incidence of SOM following treatment of 60 gray (Gy) of IMRT for Arms 1, 2, 3, and 4. FIG. 2E shows a percentage of incidence of grade 4 oral mucositis (OM) following treatment of 60 gray (Gy) of IMRT for Arms 1, 2, 3, and 4. FIG. 2F shows a percentage of patients that resolved SOM during an observation period. FIG. 2G shows a number of days before onset of SOM in patients for Arms 1, 2, 3, and 4.

FIG. 2A shows that Arm 2 and Arm 1 had a significantly lower duration of SOM than Arms 3 and 4. This indicates that a low dose of RRx-001 is more effective at lowering a duration of SOM than no dose or a high dose of RRx-001. FIG. 2B shows that Arm 1 had the lowest duration of SOM through the last treatment of IMRT. Arm 4, with no RRx-001 had the highest duration of SOM through the last treatment of IMRT. Once again, the results indicate that a low dose of RRx-001 is more effective at attenuating SOM than a high dose or no dose. Further, the study shows that any dose of RRx-001 is more effective at reducing a duration of SOM following the last IMRT treatment.

FIG. 2C shows that patients in Arm 1 had a 16 day decrease in duration of SOM through 60 Gy vs. Arm 4. Further, the low dose group of Arm 1 had the highest decrease in duration of Arm 1, Arm 2, and Arm 3. FIG. 2D shows that patients in Arm 1 had the lowest incidence of SOM through 60 Gy while Arm 4 had the highest incidence of SOM through 60 Gy. Bar graph D further shows that a low dose of RRx-001 was most effective at lowering incidence of SOM through 60 Gy while a higher dose of RRx-001 was less effective, but better than no dose. FIG. 2E shows that patients in Arm 1 had zero incidence of grade 4 OM through 60 Gy. Arm 4 had an incidence of 30% and Arms 2 and 3 had an incidence of 40% and 46% respectively. The results show that a low dose of RRx-001 had no incidence of grade 4 OM through 60 Gy. However, no dose of RRx-001 had better results than a high dose of RRx-001.

FIG. 2F shows that patients in Arm 1 had the highest incidence of resolution of SOM during the observation period and Arm 4 had the lowest incidence of resolution. FIG. 2F further shows that a low dose of RRx-001 had the greatest increase in SOM resolution vs. no dose. FIG. 2G shows that patients in Arm 1 took an average of 12 days longer than Arm 4 before onset of SOM beginning from the start of chemotherapy. Arm 1 had the largest increase, which generally trends with the other bar graphs in that the low dose of RRx-001 had greater efficacy in mitigating adverse events than a high dose.

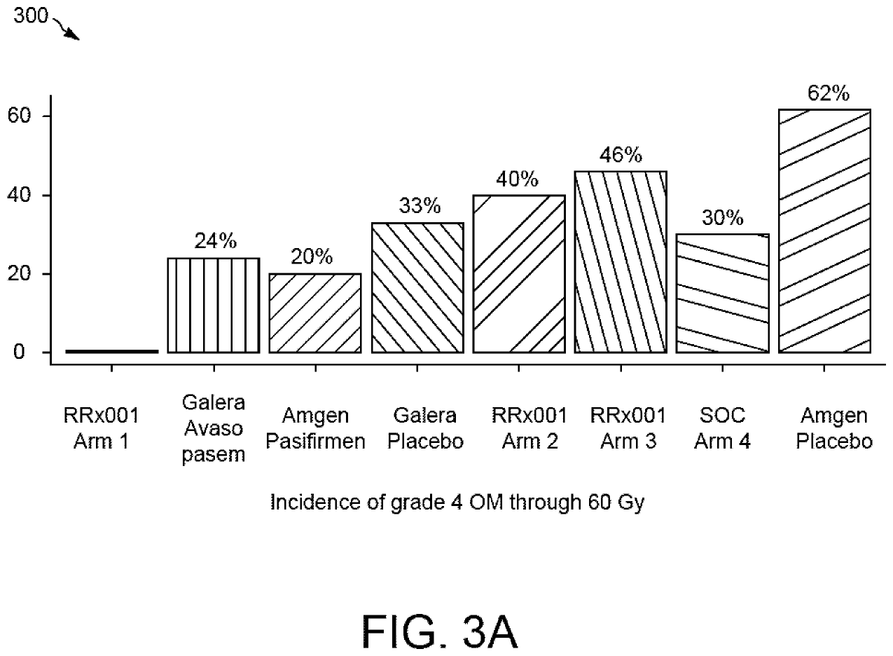

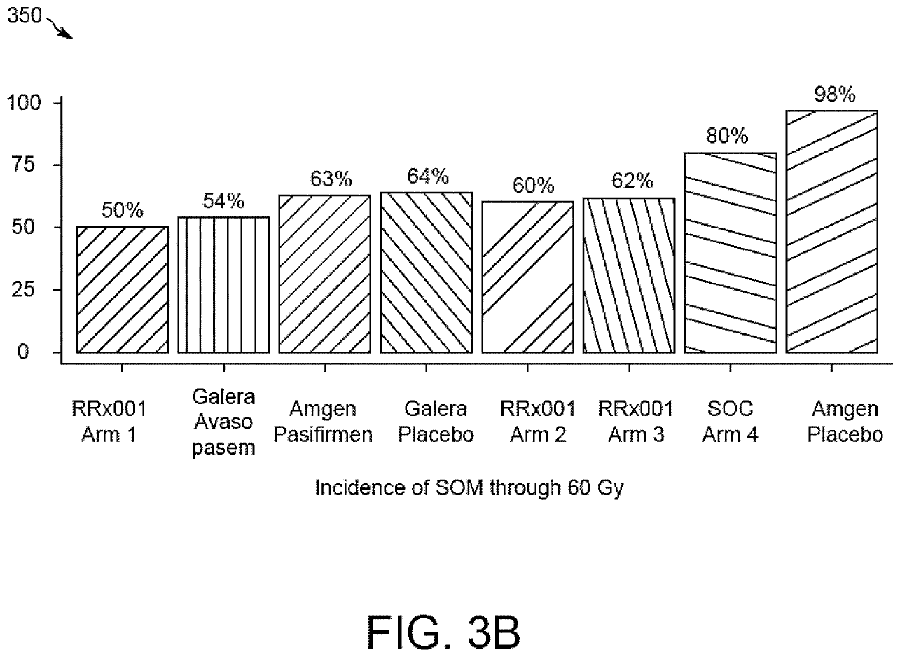

Referring to FIG. 3A. FIG. 3A shows a bar graph 300 showing incidence of grade 4 OM through 60 Gy for Arms 1, 2, 3, and 4 and various therapeutic agents that are available. The bar graph 300 shows that patients in Arm 1 of the study, which had zero incidence of grade 4 OM through 60 Gy, was the most effective treatment for reducing incidence of grade 4 OM after radiation treatment of 60 Gy.

Referring to FIG. 3B. FIG. 3B shows a bar graph 350 showing incidence of SOM through 60 Gy for Arms 1, 2, 3, and 4 and various therapeutic agents that are available. The bar graph 350 shows that patients in Arm 1 of the study had a lowest incidence of SOM after radiation treatment. The low dose RRx-001, which was given to patients in Arm 1, was more effective in both comparisons shown in bar graph 300 and bar graph 350 at reducing adverse side effects after radiation therapy.

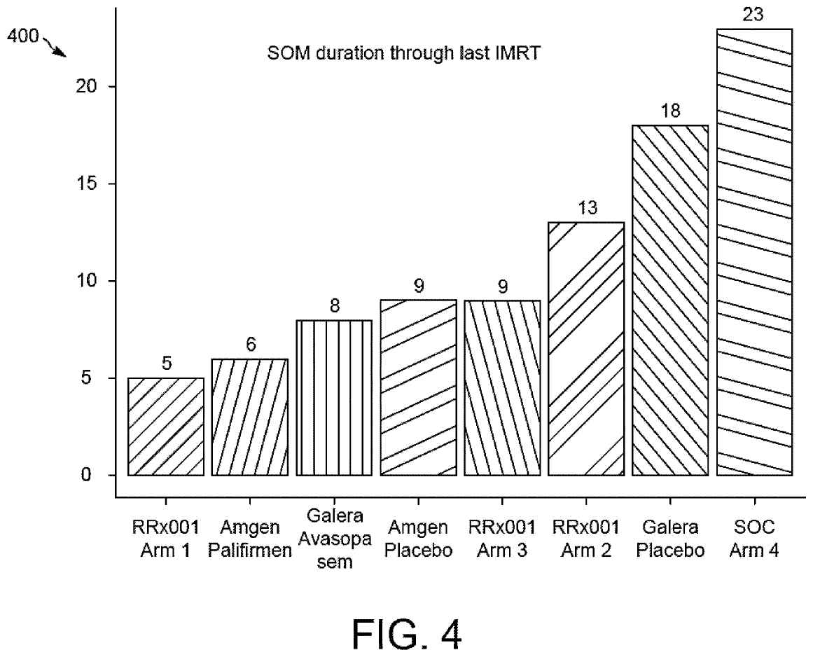

Referring to FIG. 4, FIG. 4 is a bar graph 400 showing duration of SOM after the last treatment of IMRT for Arms 1, 2, 3, 4, and various therapeutic agents that are available. The bar graph 400 shows that patients in Arm 1 experienced an 18 day decrease in SOM following treatment of IMRT compared to Arm 4, which is the standard of care. The 18 day decrease for Arm 1 patients was also the largest decrease when compared to competing regimens in the study shown in the bar graph 400.

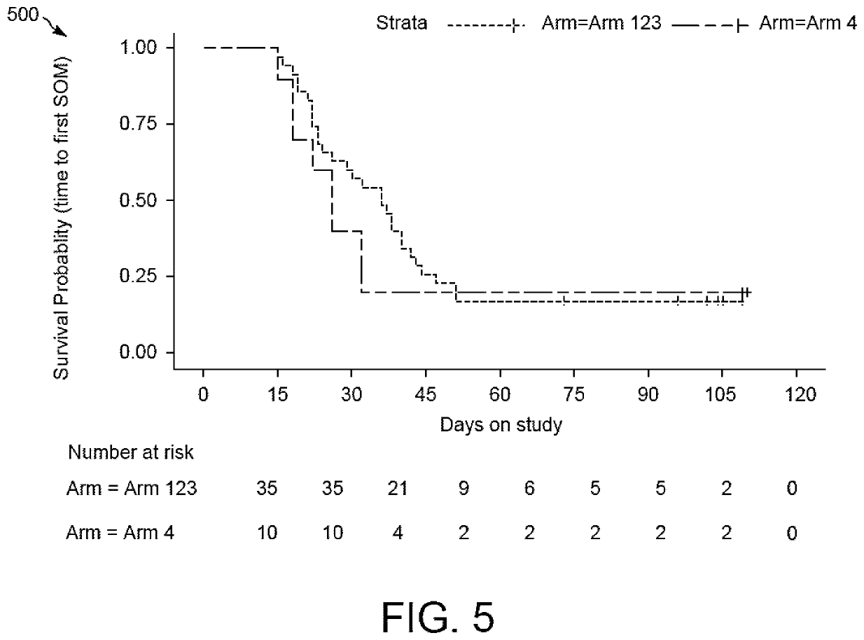

Referring to FIG. 5, FIG. 5 is a Kaplan-Meier survival curve 500 showing a probability of a patient in Arm 4 or collectively Arms 1, 2, or 3 of developing SOM after a start of chemotherapy. The Kaplan-Meier survival curve 500 shows that patients in Arms 1, 2, and 3 generally took longer to develop SOM than patients in the SOC (Arm 4).

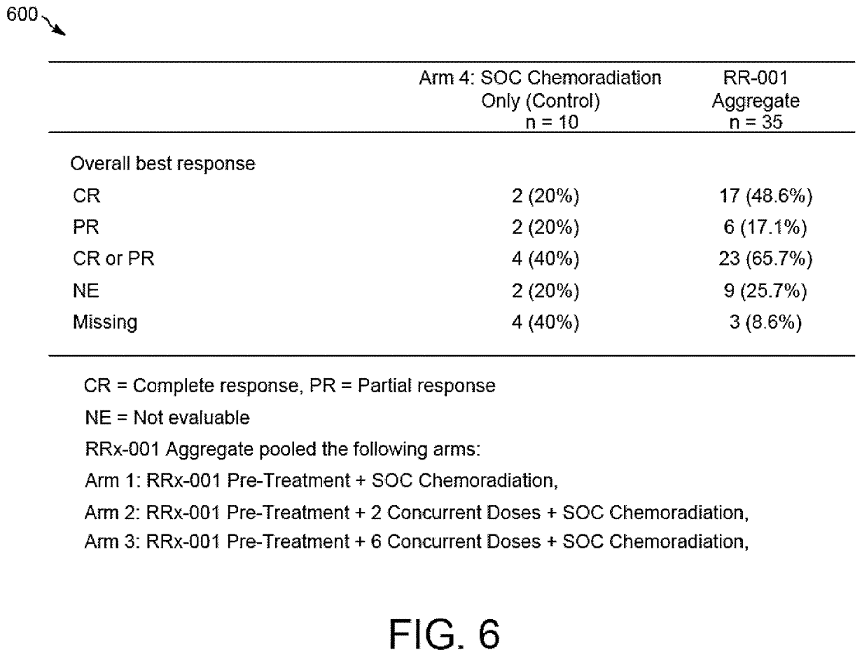

Referring to FIG. 6, FIG. 6 is a table 600 showing various incidences of outcomes for patients in Arm 4 of the PREVLAR study vs. patients in Arms 1, 2, and 3 of the PREVLAR study. The various outcomes include a complete response (CR), a partial response (PR), a CR or PR, not evaluable (NE), and missing. The table 600 shows that patients that received RRx-001 in Arms 1, 2, or 3 had an incidence of CR of 48.6% compared to 20% for patients in Arm 4 who were not administered RRx-001. Further, patients in Arms 1, 2, or 3 had an incidence of either CR or PR of 65.7% compared to 40% for Arm 4. Thus, administration of RRx-001 was correlated to better outcomes.

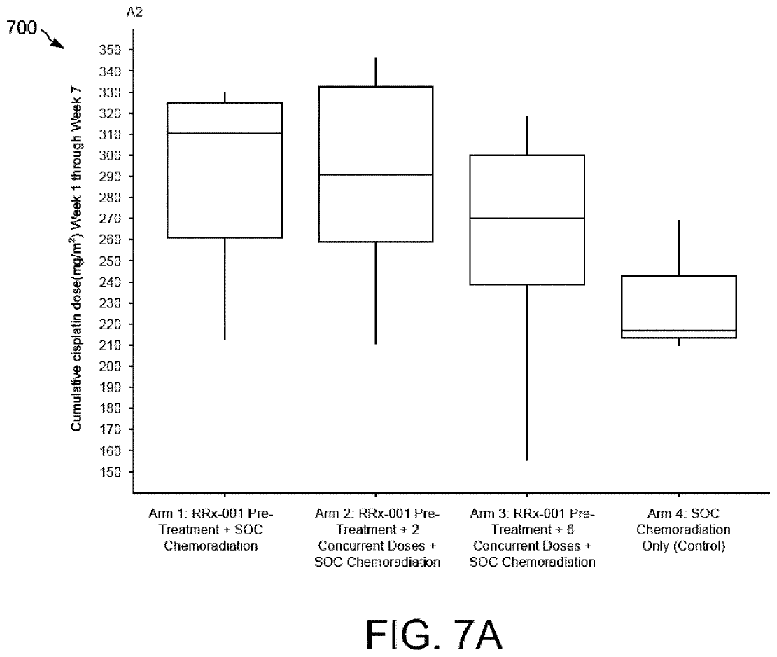

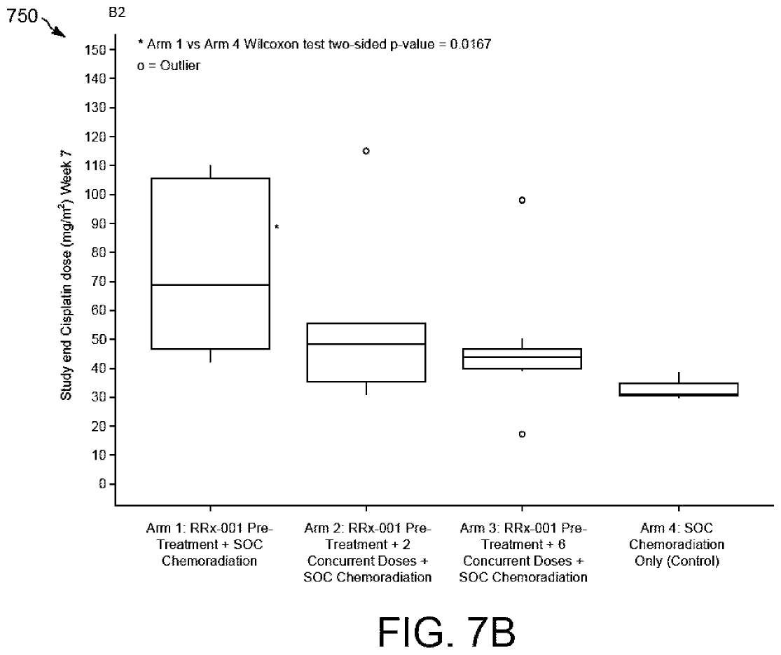

Referring to FIG. 7A, FIG. 7A is a graph 700 showing a Wilcoxon test, which shows a distribution of cumulative cisplatin dose adjusted for Body Surface Area (mg/m²) used Week 1 through Week 7 of the PREVLAR study. A higher amount of cisplatin indicates that the patients in that group were collectively able to tolerate an increased dose through the study. It is assumed that patients that reduced or stopped doses of cisplatin did so because adverse side effects made administration of cisplatin intolerable.

As shown in the graph 700, patients in Arm 1 of the study received the highest median cumulative amount of cisplatin of approximately 311 mg/m². Further, patients in Arm 4 of the study received the lowest median cumulative amount of cisplatin of approximately 219 mg/m². Accordingly, it can be concluded that patients in Arm 1, who received a low dose of RRx-001, were better able to tolerate a higher cumulative amount of chemotherapeutic agents compared to the SOC control. Patients in Arm 2 and Arm 3 of the study, who received RRx-001 concurrently with cisplatin, were better able to tolerate compared to the SOC control but did not tolerate cisplatin as well as patients in Arm 1.

Referring to FIG. 7B, FIG. 7B is a graph 750 showing a Wilcoxon test of cumulative cisplatin dose adjusted for Body Surface Area (mg/m²) at the end of the PREVLAR study. Like FIG. 7A, a higher amount of cisplatin indicates that the patients in that group were collectively able to tolerate an increased dose at the end of the study. Like the graph 700 in FIG. 7A, the patients in Arm 1 received the highest median dose of cisplatin at the end of the study and the patients in Arm 4 received the lowest median dose at the end of the study. But unlike the graph 700 in FIG. 7A, the median doses for patients in Arm 2 and Arm 3 were closer to the median dose of patients in Arm 4 than Arm 1. Thus, it may be concluded that the beneficial effect of limiting the dose of RRx-001, such as for patients in Arm 1 vs. Arm 2 and Arm 3, is more pronounced as over the time of therapeutic treatment.

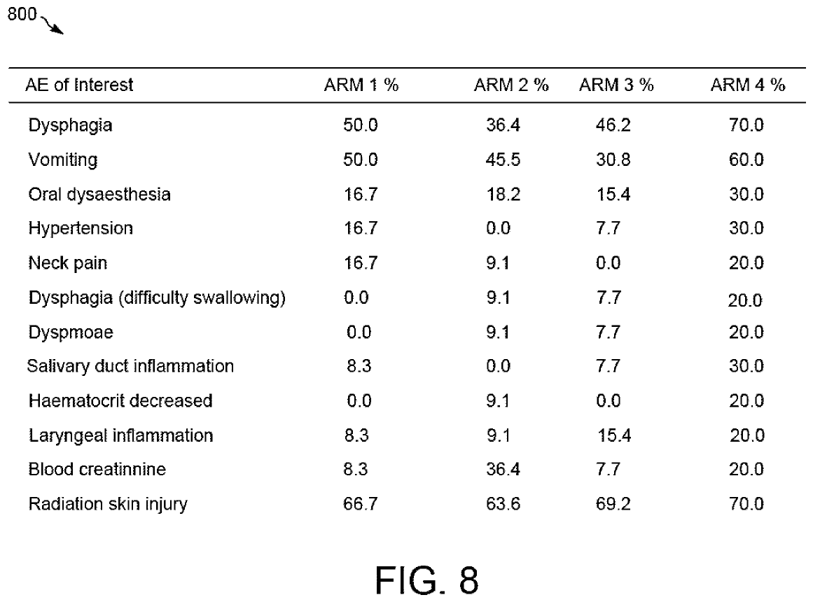

Referring to FIG. 8, FIG. 8 is a table 800 showing a frequency of various pathogenic toxicities (adverse events) observed in patients in Arm 1, Arm 2, Arm 3, and Arm 4. The various adverse events include dysphagia, vomiting, oral dysesthesia, hypertension, neck pain, dyspepsia, dyspnea, salivary duct inflammation, hematocrit decreased, laryngeal inflammation, blood creatinine increased, and radiation skin injury.

The table 800 shows that patients in Arm 4 of the study, which is standard of care delivered without RRx-001, experienced the highest frequency for all adverse events. Adverse events for which patients in Arm 1 had the lowest frequency of occurrence were dyspepsia, dyspnea, and laryngeal inflammation. Adverse events for which patients in Arm 2 bad the lowest frequency of occurrence were dysphagia, hypertension, salivary duct inflammation, and radiation skin injury. Adverse events for which patients in Arm 3 had the lowest frequency of occurrence were vomiting, oral dysesthesia, neck pain, and hypercreatinemia.

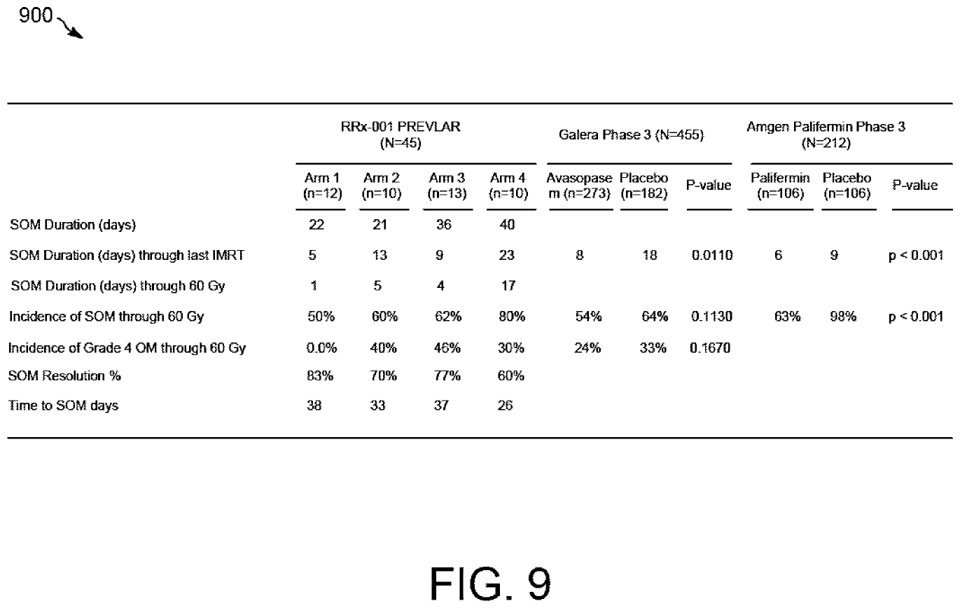

Referring to FIG. 9, FIG. 9 is a table 900 showing a summary of results related to incidence of SOM for studies comparable to PREVLAR. The comparable studies shown in the table 900 are RRx-001 PREVLAR, Galera Phase 3, and Amgen Palifermin Phase 3. The only comparable data across all studies shown in the table 900 are SOM duration (days) through the last IMRT treatment and incidence of SOM through 60 Gy. Of those, patients in Arm 1 had the lowest incidence of SOM duration after IMRT treatment and Arm 1 had the lowest incidence of SOM after receiving 60 Gy in radiation treatment.

The Galera Phase 3 study included data for incidence of grade 4 OM through 60 Gy. Of that data, patients in Arm 1 of the PREVLAR study had a zero incidence of grade 4 OM and the next lowest was Galera Phase 3 which showed a 33% incidence of grade 4 OM through 60 Gy. The data show that the low dose RRx-001 treatment is potentially effective at preventing grade 4 OM when RRx-001 is administered in a low dose according to Arm 1.

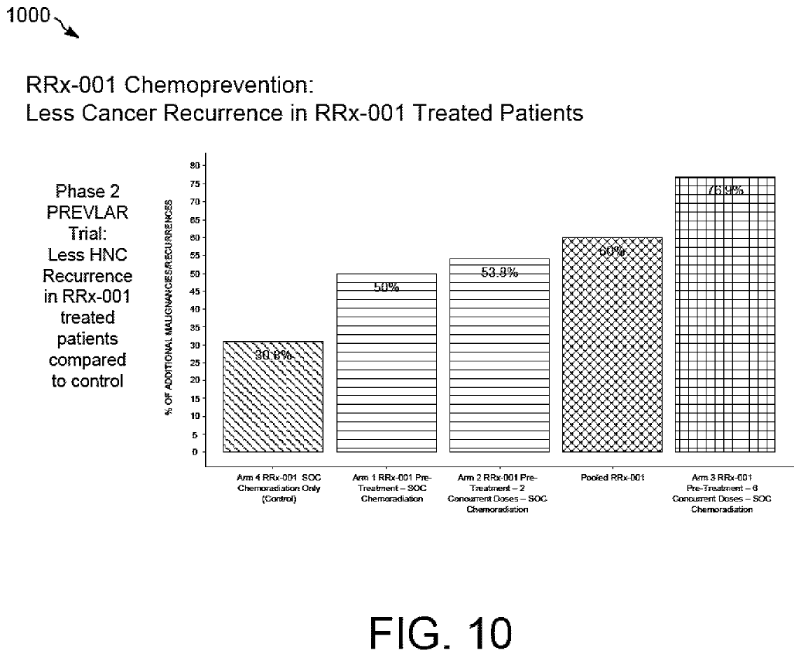

Referring to FIG. 10, FIG. 10 is a bar graph 1000 showing a percentage of less cancer recurrence in RRx-001 treated patients. Accordingly, a higher percentage is correlated to the better outcome of lower cancer recurrence. The bar graph 1000 shows that patients in Arm 3 of the PREVLAR trial had the highest percentage drop of 76.9% of cancer recurrence. Patients in Arm 4 had the lowest percentage drop of 30.8% of cancer recurrence. Patients in Arm 1 and Arm 2 of the PREVLAR trial had a 50% and 53.8% respective drop in cancer recurrence.

It may be inferred from the bar graph 1000 that a dosage amount of RRx-001 is directly correlated to lower recurrence of cancer. This result contrasts with some data, such as for OM, where a low dose of RRx-001 was correlated with better outcomes related to adverse events associated with treatment. Thus, an ideal dosage amount of RRx-001 varies depending on multiple factors including a potential severity of adverse events and risk of cancer recurrence.

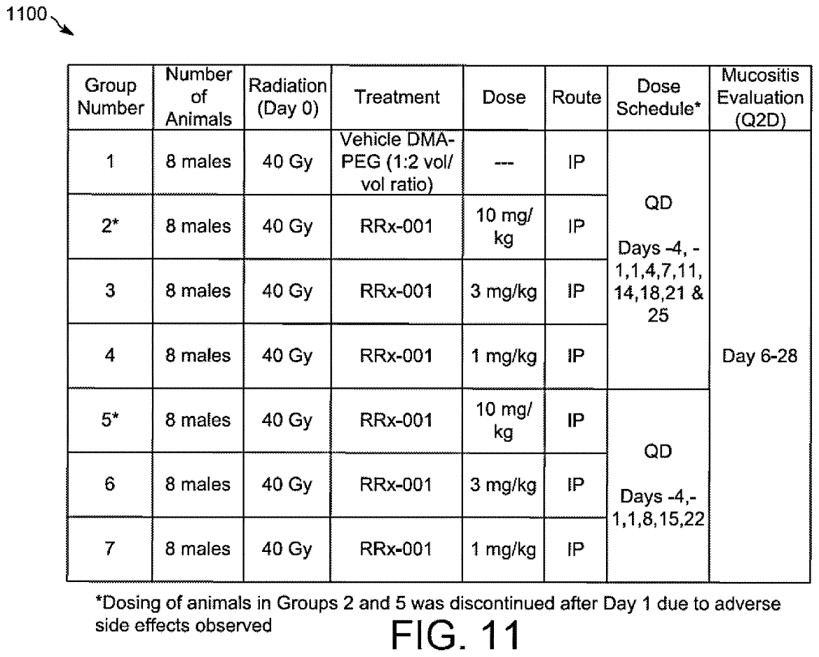

Referring to FIG. 11, FIG. 11 is a table 1100 showing a study design for an assessment of RRx-001 for the treatment of oral mucositis induced by acute radiation in hamsters. Mucositis was induced in 56 male Golden Hamsters via an acute radiation dose of 40 Gy directed to their left buccal cheek pouch. The hamsters were administered RRx-001 at a dose indicated in the table 1100. RRX-001 was administered with a vehicle comprising a 1:2 vol; vol ratio of N,N-Dimethylacetamide/40% Polyethylene Glycol (DMA:PBG). Dosing for hamsters in groups 2 and 5 was discontinued after day 1 due to adverse side effects. Otherwise, dosing was administered according to the “Dose Schedule” column of the table 1100. The study lasted 28 days. Mucositis was evaluated in hamsters from day 6 through day 28.

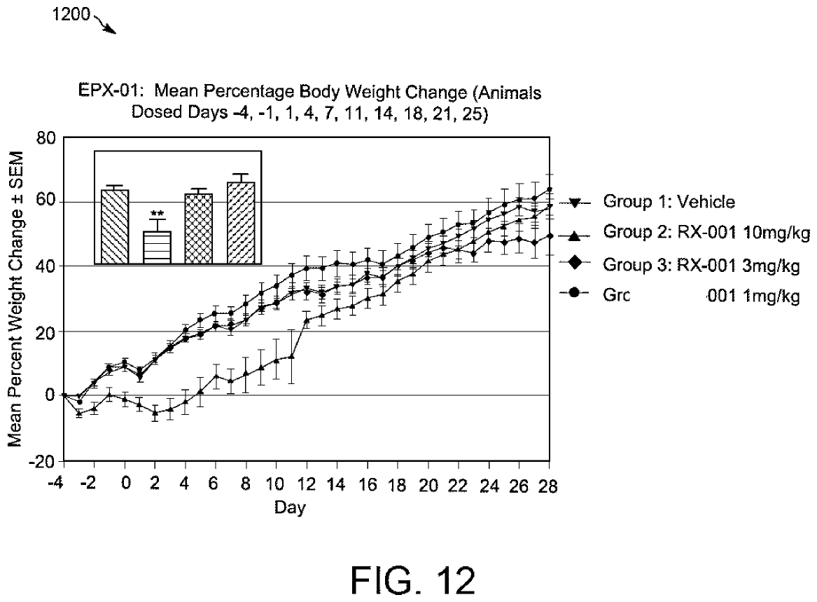

Referring to FIG. 12, FIG. 12, is a graph 1200 of the mean weight change for hamsters in groups 1~4 beginning 4 days before the study and through the study. All hamsters except those in groups 2 and 5 gained weight throughout the study. The bar graph 1205 in the top left of the graph 1200 shows a mean weight change for hamsters in groups 1, 2, 3, and 4 from left to right over the course of the study based on the area under the curve in the graph 1200.

As shown in the bar graph 1205, hamsters in groups 2 and 5 initially lost weight, but rebounded back to be in line with the other groups in the study. Hamsters in group 4 had the highest mean weight gain on day 28 and hamsters in group 3 had the lowest mean weight gain on day 28.

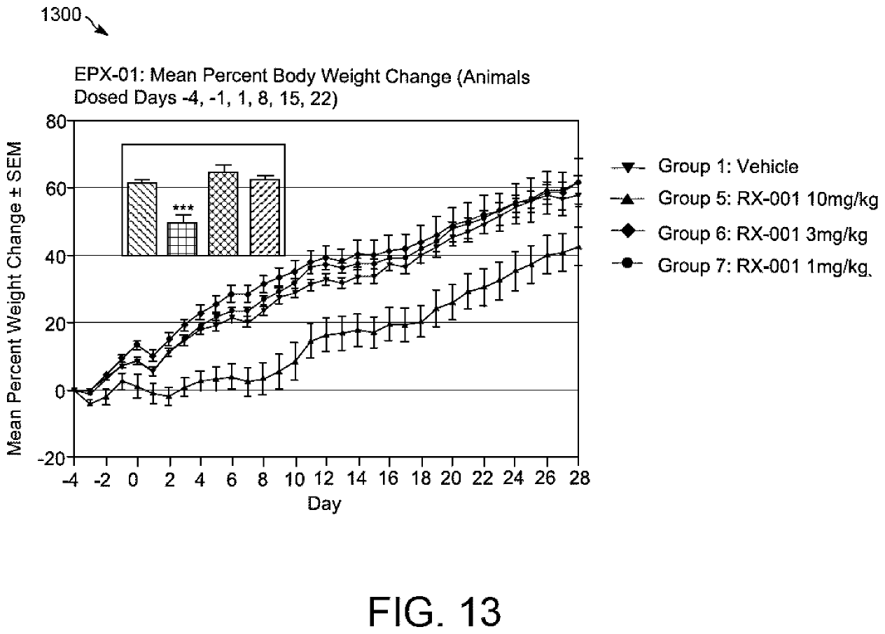

Referring to FIG. 13, FIG. 13 is a graph 1300 of the mean weight change for hamsters in groups 1 and 5-7 beginning 4 days before the study and through the study. Hamsters in groups 5-7 had a different dosing schedule than groups 2-4 that accumulates to half the total number of doses. The bar graph 1305 in the top left of the graph 1300 shows a mean weight change for hamsters in groups 1, 5, 6, and 7 from left to right over the course of the study based on the area under the curve in the graph 1300. The hamsters in groups 6 and 7 gained weight similarly to groups in the graph 1200 shown in FIG. 12.

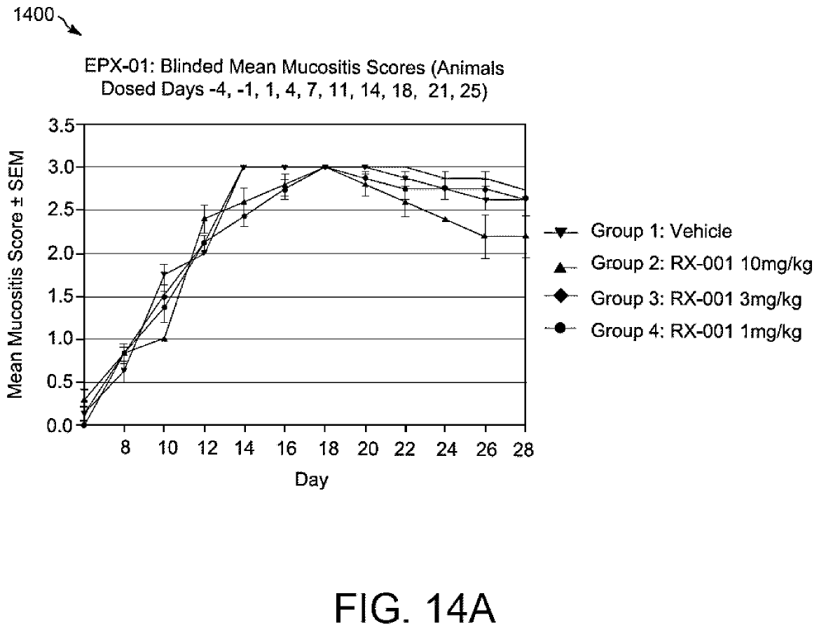

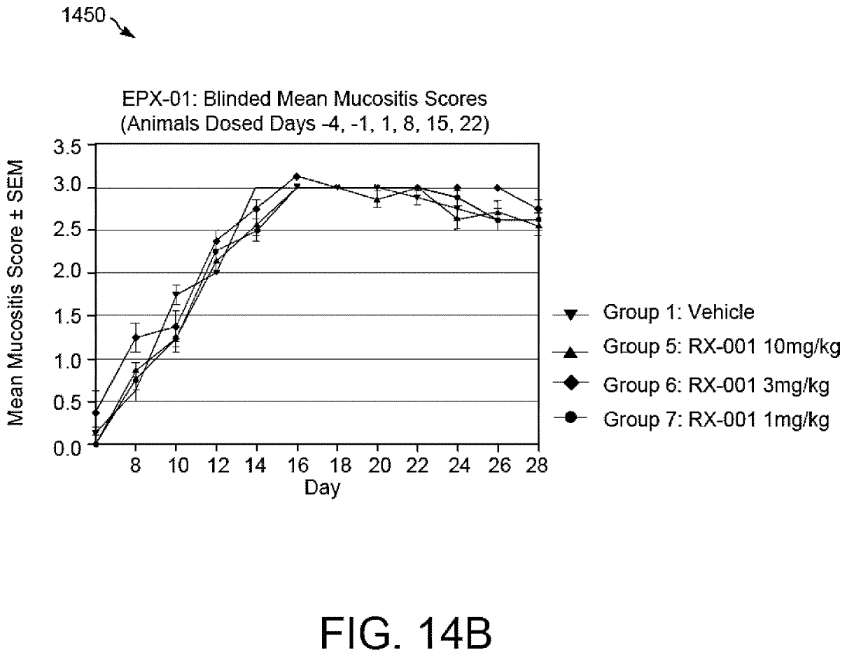

Referring to FIG. 14A and FIG. 14B, FIG. 14A shows a graph 1400 of mean daily mucositis scores for hamsters in groups 1-4. Likewise, FIG. 14B shows a graph 1450 of mean daily mucositis scores for hamsters in groups 1 and 5-7. Starting at day 6 of the study and continuing every second day thereafter (day 8, 10, 12, 14, 16, 18, 20, 22, 24, 26, and 28), the hamsters were photographed and evaluated for mucositis scoring. The hamsters were anesthetized and the left pouch everted to evaluate a score each hamster based on a severity of mucositis. The scores, on a scale of 0-5, are defined as follows:
“0” means a pouch is completely healthy.
“1” means light to severe erythema and vasodilation and no erosion of mucosa.
“2” means severe erythema and vasodilation. Erosion of superficial aspects of mucosa leaving denuded areas. Decreased stippling of mucosa.
“3” means formation of off-white ulcers in one or more places. Ulcers may have a yellow/gray color due to pseudomembrane. Cumulative size of ulcers should equal less than or equal to ¼ of the pouch. Severe erythema and vasodilation.
“4” means a cumulative seize of ulcers should equal about ½ of the pouch. Loss of pliability. Severe erythema and vasodilation.
“5” means virtually all of pouch is ulcerated. Loss of pliability (pouch can only partially be extracted from mouth).

The graph 1400 shows that hamsters in group 2 have the lowest mean daily mucositis score on day 28 of the study with a score of approximately 2.2. Group 3 has the highest number in the graph 1400 with a score of approximately 2.7. The graph 1450 in FIG. 14B has more closely grouped results and shows that hamsters in group 5 have the lowest mean daily mucositis score on day 28 of the study with a score of approximately 2.6. Group 6 has the highest mean daily mucositis score of approximately 2.7 on day 28.

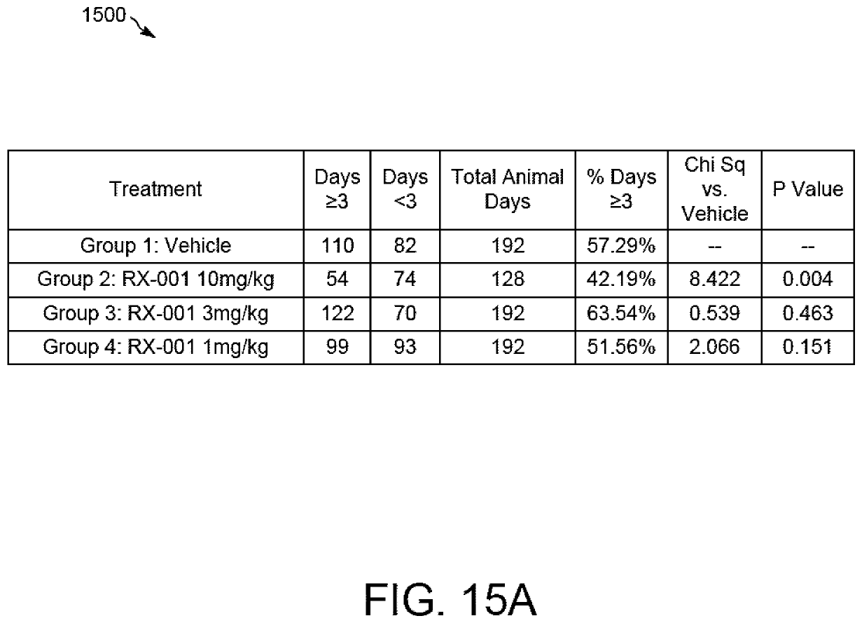

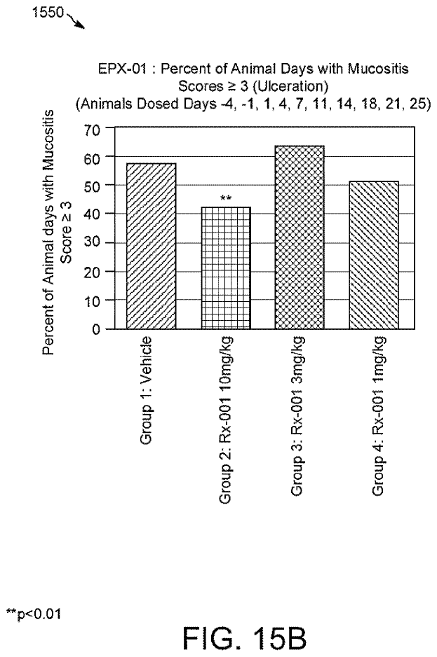

Referring to FIG. 15A and FIG. 15B, FIG. 15A is a table 1500 showing the number of days in which hamsters in groups 1-4 exhibited an elevated mucositis score of greater than or equal to 3. FIG. 15B is a bar graph 1550 showing a percent of cumulative animal days with mucositis scores greater than or equal to 3 for days 6-28. Statistical significance of the observed differences was calculated using chi-squared analysis.

As shown in the table 1500, collected data for hamsters in group 2 has the highest statistical difference from vehicle group 1. The bar graph shows that hamsters in group 2 have the lowest percent of cumulative animal days with a mucositis score of greater than or equal to 3. Hamsters in group 3 have the highest cumulative percentage of 63.54% of animal days with a mucositis score of greater than or equal to 3.

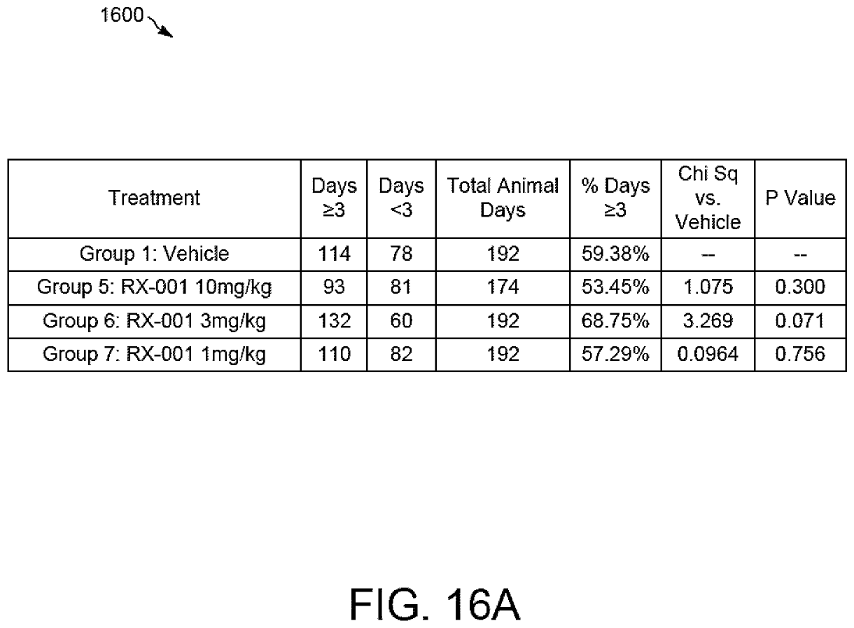

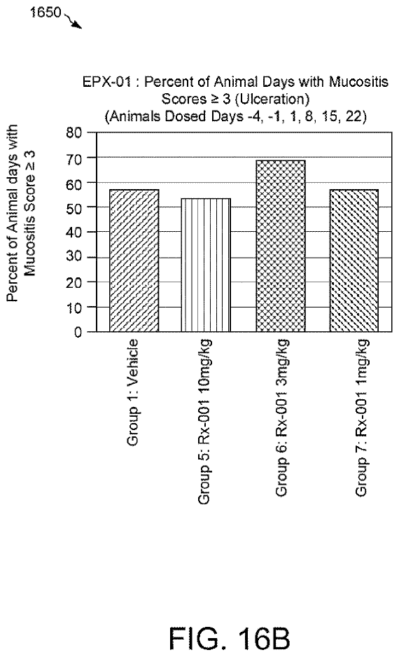

Referring to FIG. 16A and FIG. 16B, FIG. 16A is a table 1600 showing the number of days in which hamsters in groups 1 and 5-7 exhibited an elevated mucositis score of greater than or equal to 3. FIG. 16B is a bar graph 1650 showing a percent of cumulative animal days with mucositis scores greater than or equal to 3 for days 6-28. Statistical significance of the observed differences was calculated using chi-squared analysis.

As shown in the table 1600, collected data for hamsters in group 6 has the highest statistical difference from vehicle group 1. The bar graph 1650 shows that hamsters in group 6 have the lowest percent of 53.45% for cumulative animal days with a mucositis score of greater than or equal to 3. Hamsters in group 6 have the highest cumulative percentage of 63.54% of animal days with a mucositis score of greater than or equal to 3.

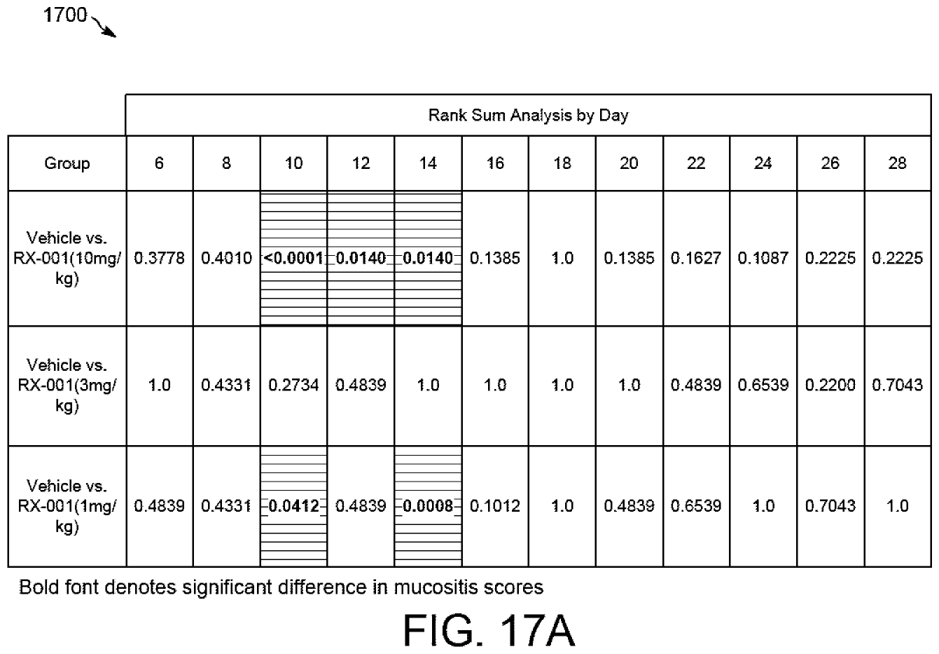

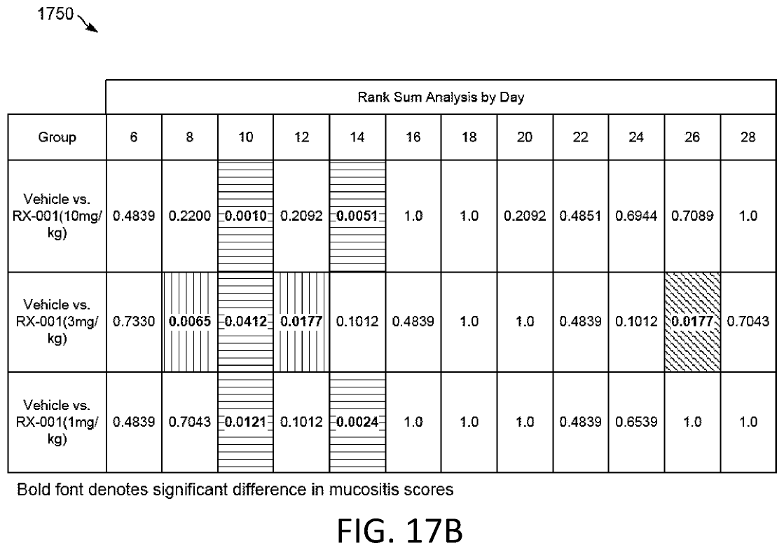

Referring to FIG. 17A and FIG. 17B, FIG. 17A is a table 1700 comparing daily mucositis scores for groups 2-4 with group 1 using a Mann-Whitney rank sum test for each day. Likewise, FIG. 17B is a table 1750 comparing daily mucositis scores for groups 5-7 with group 1 using the Mann-Whitney rank sum test for each day. The p-values for each calculation are shown. Horizontal shading denotes a decrease in mucositis scores (improvement in disease) and vertical shading denotes an increase in mucositis scores (worsening of disease).

From the top down, the first row in the table 1700 compares daily data collected from group 1 with group 2. The second row in the table 1700 compares daily data collected from group 1 with group 3. And the third row in the table 1700 compares daily data collected from group 1 with group 4. The first row in the table 1750 of FIG. 17B compares daily data collected from group 1 with group 5. The second row in the table 1750 of FIG. 17B compares daily data collected from group 1 with group 6. And the third row in the table 1750 compares daily data collected from group 1 with group 7.

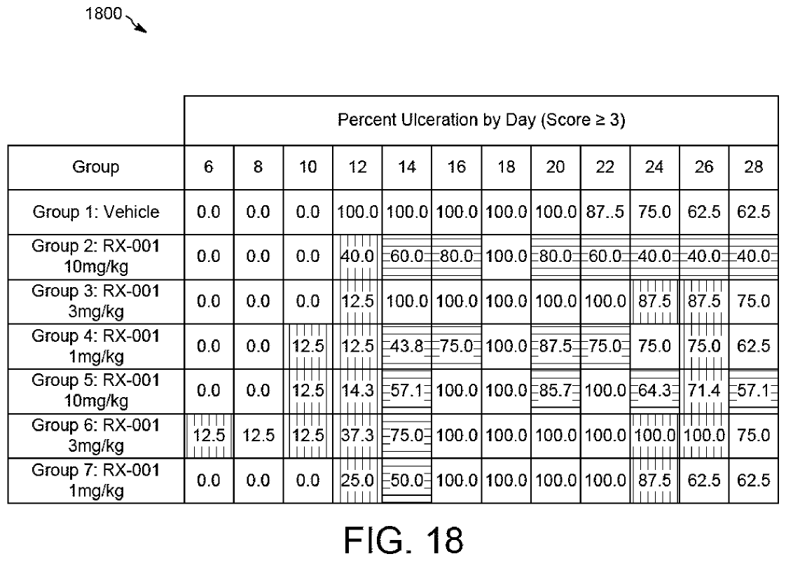

Referring to FIG. 18, FIG. 18 is a table 1800 showing a percent of hamsters with ulceration by day with mucositis scores of greater than or equal to 3. Each data point represents a percentage of hamsters in each group on a day that had both a mucositis score of 3 or greater and open ulcers. Like FIGS. 17A and 17B, horizontal shading denotes a decrease in mucositis scores and vertical shading denotes an increase in mucositis scores.

Interestingly, the animals in group 2 exhibit the worst initial result on day 12 of 40% ulceration and increasing mucositis score; and group 2 also exhibits the best result at the end of the study on day 28. On day 28 group 2 hamsters have 40% ulceration and improving mucositis scoring. The second-best results on day 28 include the hamsters in group 5, which have an ulceration of 57.1% and improving mucositis scoring.

Also of interest is that groups 3 and 6 have the worst ending result on day 28 of 75.0% ulceration and worsening mucositis scoring. Groups 4 and 7, which received a lower dose of RRx-001 of 1 mg/kg, had a middle result of 62.5% ulceration, which was better than the groups that received 3 mg/kg of RRx-001 and worse than the groups that received a high dose of 10 mg/kg. It should be noted that groups 2 and 5, which started with a high dose of 10 mg/kg RRx-001, discontinued the dose after day 1. Thus, the best results were achieved through starting with a high dose of RRx-001 and then discontinuing RRx-001 treatment.

### EXAMPLES

In order that this disclosure may be more fully understood, the following Examples are set forth. It should be understood that these examples are for illustrative purposes only and are not to be construed as limiting this disclosure in any manner.

### Example 1. Non-Clinical Studies

i. Binding to Hemoglobin

¹⁴C-RRx-001 (1 μM and 20 μM) was incubated at a temperature of 37° C. for a time period of 30 minutes with blood pooled from 3 male Sprague Dawley rats, 3 male beagle dogs, and 2 male cynomolgus monkeys, and blood not pooled from 3 individual male humans. The total covalently bound to hemoglobin (%) was calculated from 14C-RRx-001 bound to hemoglobin (pmol/mg), literature reported hemoglobin concentration (mg/mL of blood), and 14° C.-RRx-001 concentrations in blood. ¹⁴C-RRx-001-derived radioactivity, determined with a liquid scintillation counter, was found to highly partition into red blood cells and covalently bind to hemoglobin. The range of binding to the 3 different human hemoglobin varied from 24% to 34%.

ii. Binding to BCys93n

Commercial human hemoglobin was incubated with RRx-001 in buffer at 3 concentrations: 0 mM, 0.53 mM, and 5.5 mM. The precipitated protein was reduced, treated with acrylamide (to protect residual cysteines), and proteolyzed with trypsin to yield smaller peptide fragments. Liquid chromatography/mass spectrometry/mass spectrometry was used to separate and identify the tryptic peptides.

Reversed phase high-performance liquid chromatograms of the trypsin digest revealed that only one RRx-001-related alkylated peptide increased in a dose-dependent manner, correlating with a decrease in the amount of native tryptic peptide on the cysteine-93 locus of the hemoglobin β chain, which represents the preferred reaction site for small electrophilic species, like RRx-001.

RRx-001 reacted nonenzymatically and covalently with reduced glutathione (GSH), and with the β93Cys residue of hemoglobin, forming RRx-001-GSH and RRx-001-hemoglobin metabolites, respectively. Given the rapid conversion of the RRx-001 parent molecule to GSH and hemoglobin conjugates, RRx-001-GSH, being the predominant measurable metabolite in the plasma, was chosen as a surrogate for drug exposure. Terminal half-life of RRx-001-GSH was calculated as approximately 30 minutes. The area under the plasma concentration-time curve and maximum plasma concentration were mostly dosed proportional for doses up to 55 mg/m² in phase 1.

iii. RRx-001 Induces Hemoglobin Oxidation

Two samples of blood were collected from healthy humans and the hemoglobin was purified. One sample was used as a control and the other sample was incubated with 5 mg RRx-001. To measure the conversion of ferrous (Fe²⁺) oxyhemoglobin to ferric (Fe³⁺) methemoglobin, the absorbance spectra between 400 nm and 700 nm was collected at regular intervals (every 5 minutes) over a time period of about 600 minutes (10 hours) at room temperature. Oxidation rates were estimated by plotting the ratio of absorbances at 570 nm and 630 nm versus time.

iv. RRx-001 Activates Nuclear Related Factor 2 (Nrf2)

RRx-001 is an electrophilic stress regulator with anti-oxidative/anti-inflammatory, vasodilatory, and cardioprotective properties. These effects are mediated by Nrf2 activation and NLRP3 inhibition, as well as nitric oxide generation under hypoxia. NLRP3 is expressed in immune cells, especially in dendritic cells and macrophages and acts as a constituent of the inflammasome. See Ghafouri-Fard (2022) Front. Immunol. 13:926895. NLRP3 acts as a pattern recognition receptor identifying pathogen-associated molecular patterns and also recognizes damage-associated molecular patterns. See Ghafouri-Fard (2022) Front. Immunol. 13:926895. Triggering of NLRP3 inflammasome by molecules ATP released from injured cells results in the activation of the inflammatory cytokines IL-1β and IL-18. Abnormal activation of NLRP3 inflammasome stimulates inflammatory or metabolic diseases. See Ghafouri-Fard (2022) Front. Immunol. 13:926895. As such, NLRP3 is a target for decreasing activity of NLRP3 inflammasome. RRx-001 ameliorates inflammatory diseases by acting as a potent covalent NLRP3 inhibitor. See Y. Chen, et al. (2021) Cell Mol. Immunol. 18(6):1425-2436; and M. Ma, et al. (2021) Front. Immunol. 12:718779. Thus, RRx-001 is thought to be an inhibitor of not only cancer, but also inflammatory conditions.

Squamous cell cancer VII cells treated in vitro with RRx-001 (2 μM or 5 M) showed an increase in Nrf2. Mice bearing SCC VII xenografts treated with RRx-001 (10 mg/kg) showed an increase in both cytoplasmic and Nrf2 protein. Nrf2-mediated activation of protective antioxidant enzymes, heme oxygenase-1 and NAD (P) H quinone dehydrogenase 1, was upregulated following RRx-001 exposure.

### V. RRx-001 Radioprotection of Normal Cells

Mice were treated with vehicle or 10 mg/kg RRx-001 intraperitoneally 24 hours prior to irradiation (9.35 Gy delivered at 0.6 Gy/min). Survival was evaluated over 30 days post-exposure and was significantly increased in the RRx-001-treated mice (67%) compared to vehicle-treated mice (33%) (p<0.005). In addition, RRx-001-treated mice showed accelerated recovery of bone marrow cellularity and colony-forming units compared to vehicle-treated mice after a sublethal total body radiation exposure (7 Gy delivered at 0.6 Gy/min). In another study, RRx-001 treatment enhanced intestinal stem cell (crypt cell) survival and regeneration in mice that were exposed to total body irradiation of 10 to 15 Gy in combination with RRx-001 (10 mg/kg intraperitoneally) as compared to mice treated with radiation alone.

vi. Survival after Lethal Total Body Irradiation

CD2F1 mice were treated with lethal dose 70/30 whole body irradiation dose of 9.35 Gy at 0.6 Gy/min using a ⁶⁰Co source. Twenty-four hours prior to irradiation, all mice were intraperitoneally injected with either 10 mg/kg RRx-001 or the vehicle control (5% dimethyl sulfoxide in sterile water). Survival improvement in favor of pretreatment with 1 dose of 10 mg/kg RRx-001 over vehicle control irradiated mice was highly significant with an approximate 33.4% reduction in the 30-day death risk. Further, 10 mg/kg RRx-001 administered 24 hours prior to a lethal total body irradiation dose not only significantly increases survival by 33.4%. but also significantly increases the mean survival time by 7 days compared to the vehicle control.

vii. Effects on Bone Marrow after Sublethal Total Body Irradiation

Mice were irradiated with a sublethal total body irradiation dose of 7 Gy with and without a single dose of RRx-001 pretreatment 24 hours before irradiation, and a histopathological analysis of bone marrow sternebrae was performed. Following irradiation, a loss in bone marrow cellularity was observed in both the RRx-001- and vehicle-treated groups. By Day 7 an increase in cellularity was observed in the RRx-001-treated mice compared to the vehicle control. Pretreatment with RRx-001 accelerated hematopoietic recovery compared to control by Day 14. The irradiated vehicle-treated group showed a loss of bone marrow cellularity with an increase in infiltration by adipocytes compared to the irradiated RRx-001-treated group.

viii. RRx-001 Toxicology

Repeated-dose toxicity studies of RRx-001 (3 times/week up to 4 weeks in duration) were conducted in rats at dose levels of 12 mg/kg, 20.1 mg/kg, and 30 mg/kg per day and in dogs at dose levels of 4 mg/kg, 8 mg/kg, and 12 mg/kg per day (due to clinical signs, the 8 mg/kg and 12 mg/kg doses were reduced to 3 mg/kg and 5 mg/kg on Day 4). The no observed adverse effect level was <12 mg/kg in rats and 5 mg/kg in dogs. Effects on lungs were observed in dogs.

### Example 2. Phase 2a Randomized Trial to Assess the Safety and Efficacy of RRx-001 in the Attenuation of Oral Mucositis in Patients Receiving Head and Neck Chemoradiotherapy (PREVLAR)

Example 2 is a Phase 2a randomized, multi-institutional (n=13, of which 12 enrolled), open-label/unblinded, controlled trial in which patients were centrally randomized into four groups (“Arms”), with approximately 10 patients per Arm (or group) before the start of CRT, stratified by oropharynx versus oral cavity site. See M. Bonomi, et al. (2023) Int. J. Radiat. Oncol. Biol. Phys. 116(3): p. 551-559.

Study participants were adults (>18 years) with pathologically diagnosed stage III to IVB locally advanced (nonmetastatic) squamous cell carcinoma of the oral cavity or oropharynx that were scheduled to receive definitive or postoperative daily fractionation IMRT of 2.0 Gy to 2.2 Gy for a total dose of 60 Gy to 72 Gy with concomitant cisplatin given as either weekly (40 mg/m²) or tri-weekly (100 mg/m²) infusions. Radiation fields included at least 2 oral sites at risk of OM (e.g., buccal mucosa, floor of mouth, lateral/ventral tongue, or soft palate), which were planned to receive a minimum cumulative dose of 55 Gy. Table 1 demonstrates patient demographics for Example 2. Patients received a pretreatment evaluation including complete history, dental assessment, physical examination, complete blood count and complete metabolic profile, hematology and biochemistry profiles, optional laryngoscopy, chest computed tomography (CT) or positron emission tomography (PET)/CT, CT, and a magnetic resonance imaging (MRI) or PET/CT of the tumor site and neck nodes.

TABLE 1
Patient Demographics

| | No. (%) of Patients | | | | |
|---|---|---|---|---|---|
| | | Arm 2: RRX-001 | Arm 3: RRX-001 | | |
| | | Pretreatment + 2 | Pretreatment + 6 | | |
| | Arm 1: RRX-001 | Concurrent | Concurrent | | |
| | Pretreatment + SOC | Doses + SOC | Doses + SOC | Arm 4: | All |
| | Chemoradiation | Chemoradiation | Chemoradiation | Chemoradiation | Patients |
| Characteristic | (n = 12) | (n = 11) | (n = 13) | Only (n = 10) | (n = 46) |

Sex

| | | | | | |
|---|---|---|---|---|---|
| Male | 10 (83.3) | 9 (81.8) | 12 (92.3) | 9 (90) | 40 (87) |
| Female | 2 (16.3) | 2 (18.2) | 1 (7.3) | 1 (10) | 6 (13) |
| Median Age, years | 59 | 58 | 61 | 60 | 60 |

Race

| | | | | | |
|---|---|---|---|---|---|
| Asian | 0 | 0 | 1 (7.7) | 1 (10) | 2 (4 3) |
| Black or African American | 1 (8.3) | 1 (9.1) | 0 | 1 (10) | 3 (6.5) |
| White | 11 (91.7) | 10 (90.9) | 12 (92.3) | 8 (80) | 41 (89.1) |
| Hispanic or Latino | 0 | 1 (9.1) | 0 | 1 (10) | 2 (4.3) |
| Non-Hispanic | 11 (91.7) | 10 (90.9) | 13 (100) | 9 (90) | 43 (93.5) |

HPV Status

| | | | | | |
|---|---|---|---|---|---|
| Unknown | 1 (8.3) | 0 | 0 | 0 | 1 (2.2) |
| Negative | 2 (16.7) | 2 (18.2) | 2 (15.4) | 0 | 6 (13) |
| Not Done | 0 | 0 | 2 (15.4) | 1 (10) | 3 (6.5) |
| Positive | 10 (83.3) | 9 (81.8) | 9 (69.2) | 9 (90) | 37 (80.4) |

Primary Tumor

| | | | | | |
|---|---|---|---|---|---|
| Oropharyngeal | 10 (83.3) | 10 (90.9) | 11 (84.6) | 9 (90) | 40 (87) |
| Oral Cavity | 2 (16.7) | 1 (9.1) | 2 (15.4) | 1 (10) | 6 (13) |

Tobacco Use History

| | | | | | |
|---|---|---|---|---|---|
| No (never) | 7 (58.3) | 7 (63.6) | 6 (46.2) | 6 (60) | 26 (56.5) |
| Yes | 5 (41.7) | 4 (36.4) | 7 (53.8) | 4 (40) | 20 (43.5) |

Alcohol Use History

| | | | | | |
|---|---|---|---|---|---|
| Missing | 4 (33.3) | 4 (36.4) | 2 (15.4) | 3 (30) | 13 (28.3) |
| Current User | 5 (41.7) | 4 (36.4) | 8 (61.5) | 6 (60) | 23 (50) |
| Former User | 3 (25) | 3 (27.3) | 3 (23.1) | 1 (10) | 10 (21.7) |

History of Smokeless Tobacco

| | | | | | |
|---|---|---|---|---|---|
| Missing | 7 (58.3) | 7 (63.6) | 6 (46.2) | 6 (60) | 26 (56.5) |
| Former User | 0 | 0 | 2 (15.4) | 1 (10) | 3 (6.5) |
| Never User | 5 (41.7) | 4 (36.4) | 5 (38.5) | 3 (30) | 17 (37) |

History of Cigar Smoking

| | | | | | |
|---|---|---|---|---|---|
| Missing | 7 (58.3) | 7 (63.6) | 6 (46.2) | 7 (70) | 27 (58.7) |
| Former Smoker | 1 (8.3) | 0 | 2 (15.4) | 0 | 3 (6.5) |
| Never Smoker | 4 (33.3) | 3 (27.3) | 5 (38.5) | 3 (30) | 16 (34.8) |

RRx-001 preferentially binds to glutathione (GSH) and to a cysteine residue on hemoglobin, leading to the displacement of nitric oxide. See B. Oronsky, et al. (2020) Oncoimmunology. 9(1):1746172. RRx-001 also binds to a reactive cysteine on the NLRP3 inflammasome, blocking its assembly and inhibiting inflammation. See Y. Chen, et al. (2021) Cell Mol Immunol. 18:1425-1436. RRx-001 induces severe oxidative cytotoxic stress to tumor cells as a result of increased carbon- and nitrogen-radicals, reductions in Myc and CD47, macrophage repolarization, and tumor apoptosis. See B. Oronsky, et al. (2019) J Cancer Res Clin Oncol. 145:2045-2050. Prior safety assessments in humans, with and without concurrent radiation therapy, concluded that RRx-001 was well-tolerated without significant toxicity. See T. Reid, et al. (2015) Lancet Oncol. 16:1133-1142. RRx-001's mechanism as an NF-κB/NLRP3 inflammasome inhibitor and an Nrf2 activator prompted this Example to interrogate its utility to mitigate OM.

Arms 1 to 3 received twice weekly infusions of RRx-001, 4 mg/dose after a premedication dose of dexamethasone (10 mg) for the 2 weeks preceding the start of CRT. Patients in Arm 2 received two additional 4 mg doses of RRx-001 after being premedicated with dexamethasone in weeks 2 and 5 of their CRT regimen. Patients in Arm 3 received weekly RRx-001 (4 mg after dexamethasone pre-dosing) during the first 6 weeks of CRT. Treatment was completed on the last day of radiation therapy (LDRT). Arm 4, the control Arm, received dexamethasone, but did not receive RRx-001.

All patients received best supportive oral care as described in the Multinational Association of Supportive Care in Cancer guidelines, 11 including standardized instructions based on the International Society of Oral Oncology Patient Care fact sheet (http://isoo.world), and oral hygiene products (soft toothbrush, floss, xylitol-containing chewing gum, fluoride-containing toothpaste, 0.4% fluoride gel) to optimize compliance.

The treatment phase commenced 2 weeks before the start of CRT and continued until the LDRT. The short-term follow-up stage started the day after LDRT and continued until complete resolution of ulcerative OM (World Health Organization (WHO) grade≤1 or 6 weeks after the LDRT, whichever came first). Long-term follow-up was performed to assess tumor response for 12 months after the LDRT. OM was assessed twice weekly beginning on the first day of CRT and continuing to resolution of ulcerative OM by trained evaluators using a standardized data collection tool.

### Efficacy

Compared to control subjects, duration of SOM between first to last radiation visit was reduced in patients treated with the RRx-001. Duration was evaluated in two ways. First, it was defined as the time from the onset of SOM to the last visit at which SOM was noted among only those patients who developed SOM. Patients who never manifested SOM were excluded. The median duration of SOM was 24 days in controls and it was 8.5 days, 17 days, and 10 days among patients in Arms 1, 2 or 3, respectively.

In a second approach, duration was determined for all patients from baseline to the last day of radiation including those cases in which a patient never developed SOM for whom a duration of 0 days was assigned. The RRx-001 administration was also associated with shortened SOM duration. Median SOM duration was 18 days in the control Arm and it was reduced to 5 days for patients in Arm 1, 13.5 days for Arm 2, and 8 days in Arm 3.

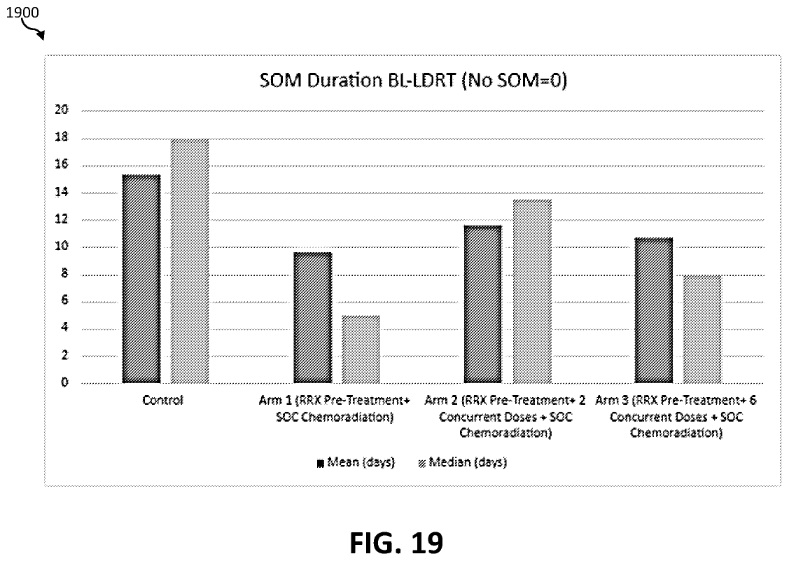

FIG. 19 is a chart 1900 associated with duration of SOM from baseline to last day of radiation, according to at least some embodiments disclosed herein. A duration of 0 days was assigned to those patients who never developed SOM. A majority of the difference in duration was attributable to the observation that the RRx-001 delayed the onset of SOM, as shown in FIG. 19. At cumulative RT doses of 45 Gy, almost two-thirds (63.6%) of patients in the control Arm already manifested SOM whereas among patients who received the RRx-001, only 42.9% of patients exhibited SOM. Likewise, while the proportion of control patients with SOM increased to 72.7% at 55 Gy and 80% at 65 Gy, corresponding increases in patients in the treated Arms was 57.1% and 71.4% respectively. By the last dose of radiotherapy (LDRT) (about 72 Gy), no differences in SOM incidence were seen between control (76.1%) and the aggregate active Arms (77.1%). Similarly, the incidence of SOM through the short-term follow-up period did not differ between control (72.7%) and active (80%) patients. Around one-third (35.4%) of all study patients experienced at least one day of Grade 4 OM between the first and LDRT. There was a small reduction in Arm 1 patients (23.1%) compared to the control Arm (33.3%). However, there no impact was seen among patients in Arms 2 (40%) or 3 (46.2%).

The incidence of SOM (any WHO score ≥3 between baseline to the last day of radiation) in the control Arm was consistent with expectations (72.7%). While the RRx-001-treated patients had a similar incidence (Arm 1 83.3%, Arm 2 70%, Arm 3 76.9%), there was a substantive difference in the percent of visits at which SOM was noted. Among patients who received a minimum of 65 Gy of cumulative radiation, SOM was reported in 34.9% of study visits amongst control patients. In contrast, the aggregate percent of SOM visits for patients in the active arms was 23.9% (Arm 1 23.6%, Arm 2 22.2%, and Arm 3 25.7%). Of the RRx-001 schedules tested, only Arm 1 appeared to be effective in reducing the percent of visits at which patients were noted to have the most severe forms (Grade 4) mucositis (control 8.1%, Arm 1 3.3%, Arm 2 12.2%, Arm 3 7.7%).

The most severe forms of OM were observed in 8.2% of visits in the SOC cohort, which was true in only 3.3% of patients in Arm 1 (p=0.056). In contrast, 12.2% of Arm 2 visits and 7.7% of Arm 3 visits manifested Grade 4 OM. As defined herein, mixed model repeated measures (MMRM) can be applied for highly variable repeated measure data and is a way to estimate the drug effect at each visit and dose, however without any assumptions regarding the dose-response shape. See G. Wellhagen, et al. (2020) Pharm Res. 37(8):157. The estimate (averaged across all visits) MMRM probability of SOM was 40.1% on SOC as compared to 15.3% on (pooled) RRx-001 treatment. Table 2 depicts MMRM analysis of SOM incidence comparing pooled RRx-001-treated patients compared to the control. Table 3 depicts MMRM analysis of SOM inferential test comparing pooled RRx-001-treated patients as compared to the control.

TABLE 2
MMRM analysis of SOM incidence comparing pooled RRx-001-treated
patients vs. control. Overall study-wide SOM incidence estimates
derived from MMRM analysis and displayed.

| | SOM Probability | | | |
|---|---|---|---|---|
| | (averaged across | | | |
| Treatment | all visits) | SE | LCL | UCL |
| SOC Chemoradioation alone | 0.401 | 0.1869 | 0.127 | 0.755 |
| Pooled RRx-001 | 0.153 | 0.0527 | 0.075 | 0.286 |

Confidence level used: 0.95 Intervals are back-transformed from the logit scale
Tests are performed on the logit scale.
SE = Standard Error,
LCL = Lower Confidence Limit,
UCL = Upper Confidence Limit

TABLE 3
MMRM analysis of SOM inferential test comparing
pooled RRx-001-treated patients vs control.

| | Odds ratio | | | UCL | z | P- |
|---|---|---|---|---|---|---|
| Contrast | estimate | SE | LCL | Null | ratio | value |
| SOC [symbol] alone/ | 3.24 | [symbol] | 2 [symbol] | 1 | 1.494 | 0.1352 |
| (Pooled RRx-001) [symbol] | | | | | | |

Confidence level used, [symbol] Intervals [symbol] back-transformed from the logit scale
Tests are performed on the logit scale.
SE = Sandard Error,
LCL = Lower Confidence Limit,
UCL = Upper Confidence Limit [symbol]
indicates data missing or illegible when filed

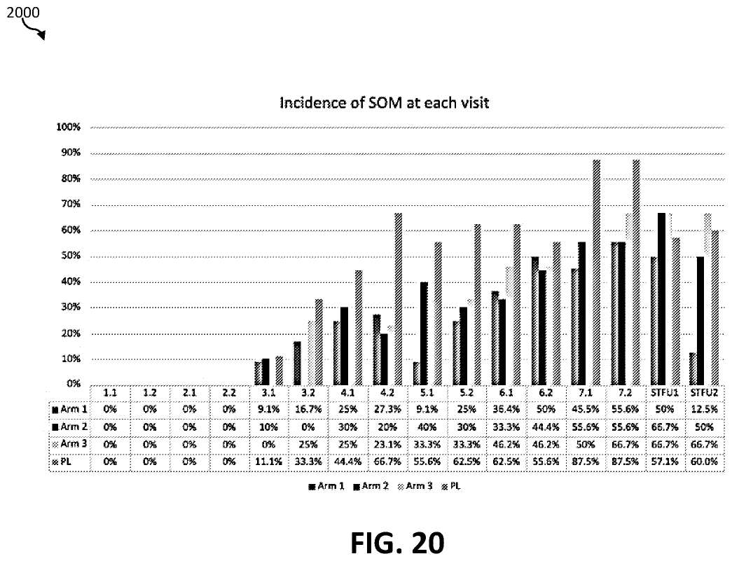

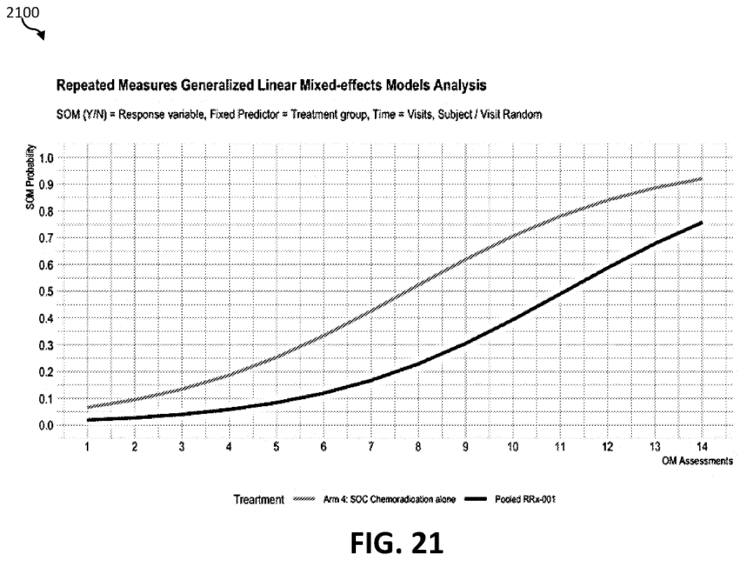

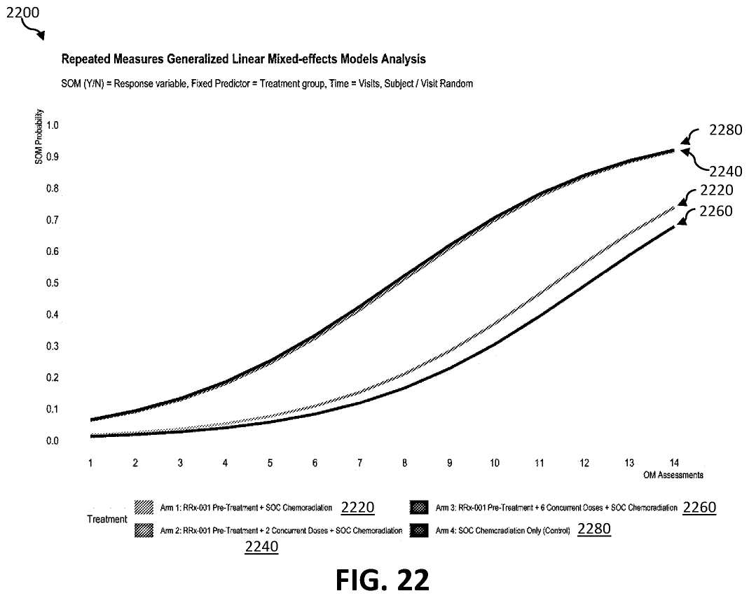

To better understand the impact of the RRx-001 on the severity and course of SOM, additional analyses were performed. FIG. 20 is a chart 2000 comparing an incidence of SOM by study visit, according to at least some embodiments disclosed herein. FIG. 21 is a graph 2100 of estimated severe oral mucositis probabilities according to MMRM by study visit and treatment group (pooled RRx-001 as compared to control), according to at least some embodiments disclosed herein. FIG. 22 is a graph 2200 of repeated measures generalized linear mixed-effects model analysis by study cohort for Arm 1 2220, Arm 2 2240, Arm 3 2260, and Arm 4 2280, according to at least some embodiments disclosed herein.

The proportional differences in WHO score severity were compared by study visits between Arms, as shown in FIG. 20. Visits were designated by week of treatment and by one of two study assessment visits each week. Thus, visit 4.1 indicates study week 4, visit 1. Radiation fractions of 2 Gy (Monday-Friday) were delivered from which cumulative radiation by study visit was extrapolated. FIG. 20 demonstrates that RRx-001-treated patients had more mild mucosal changes compared to controls. The MMRM approach was then used to ascertain a quantitative assessment of RRx-001 benefit over controls, as shown in FIG. 21. Using a generalized linear mixed effects model (GLMER) for repeated binary measures, the aggregate RRx-001 effect favorably impacted the course of SOM compared to the control Arm (p<0.0001), as shown in FIG. 22.

### Patient Reported Outcomes (Oral Mucositis Daily Questionnaire, OMDQ)

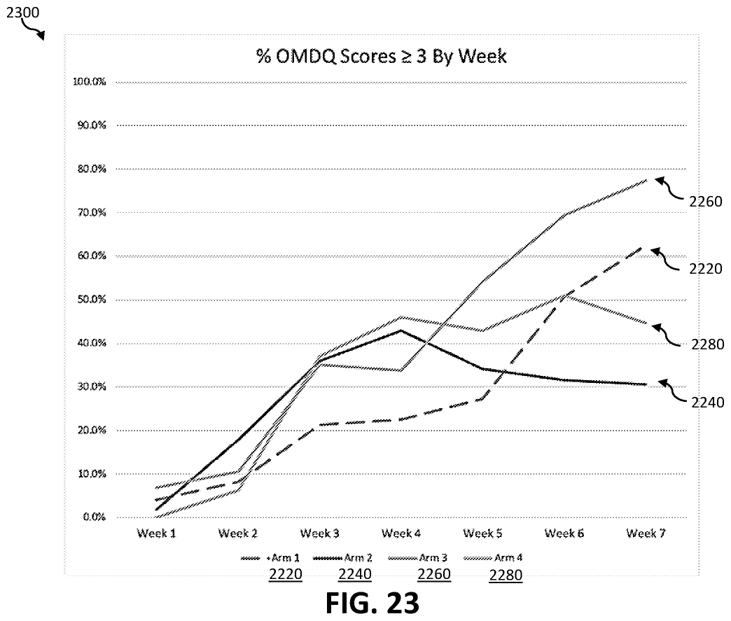

The OMDQ provided a validated platform for endpoints associated with OM and was used to evaluate symptom trajectory in the context of pain management interventions in patients being treated with CRT for head and neck cancers. The percent of patients in each Arm were evaluated during each week of treatment. The evaluation measured the severity of mouth and throat soreness based on a 0-4 scale, with a score of 3 being associated with “quite a lot of soreness” and a score of 4 being associated with “extreme soreness.” As expected, the percentage of patients reporting significant mouth and throat soreness (MTS) increased with cumulative radiation dose and peaked and plateaued in control patients around week 4 and then continued. FIG. 23 is a graph depicting extreme mouth and throat soreness scores by study week for Arm 1 2220, Arm 2 2240, Arm 3 2260, and Arm 4 2280. Arm 1 2220 patients recorded fewer visits of “quite a lot or extreme soreness” (see FIG. 23), while patients in the other treatment appeared to derive less benefit.

### Adverse Events (AEs)

The overall adverse event profile (AEs occurring in ≥10% of RRx-001-treated patients) was comparable across RRx-001 and SOC Arms, consistent with the known toxicities of IMRT plus cisplatin (i.e., nausea, vomiting, dysgeusia, radiation skin injury, fatigue, dyselectrolytemias, dry mouth, tinnitus and cytopenias) and taking into account the unbalanced allocation of RRx-001-treated patients that received cisplatin 100 mg/m² every 3 weeks as compared to 40 mg/m² every week. Of potential significance for OM, decreased rates of several pathognomonic toxicities were seen (see Table 4). The only toxicity attributed to RRx-001 was discomfort during infusion. As shown in Table 4 and Table 5, of the SAEs reported, none were attributed to RRx-001.

TABLE 4
SAEs which occurred with a frequency ≥ 10%

| | Arm 1 | Arm 2 | Arm 3 | Arm 4 |
|---|---|---|---|---|
| Dysphagia | N/A | 9.1% | 30.8% | 40% |
| Lymphocyte count decreased | 33.3% | 72.7% | 53.8% | 40% |
| Neutrophil count decreased | 8.3% | 27.3% | 30.8% | 20% |
| White blood cell count decreased | 16.7% | 36.4% | 30.8% | 10% |
| Decreased appetite | 8.3% | 9.1% | 15.4% | N/A |
| Acute kidney injury | N/A | 18.2% | N/A | N/A |
| Oropharyngeal pain | 16.7% | 9.1% | 7.7% | 20% |
| Gastrointestinal tube insertion | N/A | N/A | 15.4% | N/A |

TABLE 5
SAEs which occurred with a frequency ≥ 5%

| | Arm 1 | Arm 2 | Arm 3 | Arm 4 |
|---|---|---|---|---|
| Anemia | 8.3% | 9.1% | N/A | 10% |
| Febrile neutropenia | 8.3% | N/A | N/A | N/A |
| Leukocytosis | N/A | 9.1% | N/A | N/A |
| Atrial flutter | 8.3% | N/A | N/A | N/A |
| Deafness | N/A | 9.1% | N/A | N/A |
| Hypoacusis | 8.3% | N/A | N/A | N/A |
| Colitis | N/A | 9.1% | N/A | N/A |
| Dry Mouth | N/A | N/A | 7.7% | N/A |
| Dysphagia | N/A | 9.1% | 30.8% | 40% |
| Gastric Perforation | N/A | N/A | 7.7% | N/A |
| Nausea | 8.3% | 9.1% | 7.7% | 20% |
| Oral dysaesthesia | N/A | N/A | 7.7% | 20% |
| Oral dysesthesia | N/A | N/A | 7.7% | N/A |
| Oral pain | N/A | 9.1% | 7.7% | 10% |
| Salivary duct inflammation | N/A | N/A | 7.7% | 10% |
| Stomatitis | N/A | 9.1% | 7.7% | N/A |
| Vomiting | 8.3% | N/A | 7.7% | 20% |
| Complication associated with | N/A | N/A | 7.7% | N/A |
| device | | | | |
| Device related thrombosis | N/A | 9.1% | N/A | N/A |
| Sudden death | N/A | 9.1% | N/A | N/A |
| Candida infection | N/A | 9.1% | N/A | N/A |
| Lung infection | N/A | N/A | 7.7% | 10% |
| Respiratory tract infection | 8.3% | N/A | N/A | N/A |
| Alanine aminotransferase increased | N/A | 9.1% | N/A | N/A |
| Blood creatinine increased | N/A | 9.1% | N/A | N/A |
| Lymphocyte count decreased | 33.3% | 72.7% | 53.8% | 40% |
| Neutrophil count decreased | 8.3% | 27.3% | 30.8% | 20% |
| Weight decreased | 8.3% | N/A | 7.7% | N/A |
| White blood cell count decreased | 16.7% | 36.4% | 30.8 | 10% |
| Decreased appetite | 8.3% | 9.1% | 15.4% | N/A |
| Hypocalcemia | N/A | N/A | 7.7% | N/A |
| Hypokalemia | 8.3% | 9.1% | 7.7% | N/A |
| Hypophosphatasemia | N/A | 9.1% | 7.7% | 10% |
| Flank pain | N/A | 9.1% | N/A | N/A |
| Tumor pain | N/A | N/A | 7.7% | N/A |
| Dysgeusia | 8.3% | N/A | N/A | N/A |
| Acute kidney injury | N/A | 18.2% | N/A | N/A |
| Laryngeal inflammation | N/A | 9.1% | N/A | N/A |
| Oropharyngeal pain | 16.7% | 9.1% | 7.7% | 20% |
| Swelling face | N/A | N/A | 7.7% | N/A |
| Gastrointestinal tube insertion | N/A | N/A | 15.4% | N/A |
| Embolism | N/A | N/A | 7.7% | N/A |
| Hypertension | 8.3% | N/A | N/A | 20% |
| Hypotension | N/A | N/A | 7.7% | N/A |

### Long Term Follow-Up Tumor Response

Oral mucositis remains a significant toxicity for patients treated with concomitant chemoradiation for cancers of the head and neck. While effective preventive or interventional therapies have been elusive, mitigation of oxidative stress, activation of the innate immune response, and attenuation of pro-inflammatory signaling have shown promising results in clinical trials as potential agents make their way through the development process.

RRx-001 is unique given its protective effects under normoxia and cytotoxic effects under hypoxia. This example evaluated and compared the safety and efficacy of three dosing schedules as compared to a standard of care control. The comparative efficacy of a single pre-radiation RRx-001 dose was evaluated in comparison to multiple doses at two schedules. The results of this example demonstrated that patients who received RRx-001 for the two weeks immediately prior to the start of CRT (Arm 1) responded more favorably to treatment as compared to those patients who received additional RRx-001 dosing during CRT. Specifically, patients in Arm 1 had a shorter duration of SOM when duration (BL to LDRT) was calculated amongst only patients who developed SOM, or when a duration of zero days was assigned to patients who never developed SOM. Likewise, when the proportional incidence of SOM was determined by cumulative radiation, Arm 1 patients outperformed the other two treatment Arms. Similarly, Arm 1 patients had the fewest visits in which the most severe form of OM was seen and were less symptomatic than patients in the other treatment Arms. In contrast, patients who received pre-CRT RRx-001 which was then followed by six weeks of additional infusions (Arm 3) failed to benefit from test therapy. Patients in Arm 1 had a lower frequency of reported significant MTS (OMDQ score≥3) than controls, but they (like other treated patients) started de novo opioids later and used opioids for fewer days, than did control patients.

The superiority of RRx-001 pre-CRT dosing over the other schedules seems rather counterintuitive on its face. Typically, radioprotectants are given daily, weekly, or bi-weekly. See C. M. Anderson, et al. (2020) J Clin Oncol. 38(3):288; M. Henke, et al. (2011) J Clin Oncol. 29(20):2815-2820, and M. Kudrimoti, et al. (2016) J Biotechnol. 239:115-125. However, while pharmacokinetic data suggests that RRx-001 has a relatively short half-life of under an hour, mechanistically the biological half-life is quite extended. See J. Scicinski, et al. (2011) Drug. Metabol. Rev., Abstract P81. Though not bound by theory, it is hypothesized that pre-CRT infusion of RRx-001 serves as a preconditioning stimulus, in which a short, sublethal burst of free radical stimulation from depletion of glutathione and release of nitric oxide induces protection against the subsequent severe insult from cisplatin and IMRT. This protective effect is mediated by stimulation of nuclear factor erythroid 2-related factor 2 (NFE2L2) gene expression (a regulator of the intracellular antioxidant response) and subsequent activation of Nrf2, which controls an array of antioxidant genes. See B. Oronsky, et al. (2022) Life Sciences in Space Research 35:69-75. Simultaneous RRx-001 suppression of KEAP1 (a tumor suppressor gene and a metastasis suppressor gene) inhibits its interference with Nrf2 activation. See N. Wakabayashi, et al. (2003) Nat Genet. 35(3):238-245.

IKKα is an enzyme complex that is involved in propagating the cellular response to inflammation. Nuclear factor kappa-light-chain-enhancer of activated B cells (NF-κB) is a protein complex that controls transcription of DNA, cytokine production and cell survival. NF-κB is found in almost all animal cell types and is involved in cellular responses to stimuli. RRx-001 interferes with NLRP3-mediated inflammasome activation, shifts the hemoglobin-oxygenation curve to the left, favoring (2 binding to hemoglobin at lower oxygen tension, which protects against ionizing radiation, and binds IKKα to interfere with NF-κB activation and the resultant pro-inflammatory cytokine cascade that is associated with mucosal damage. See J. Wei, et al. (2019) Biomed Pharmacother. 118:109217.

The superiority of Arm 1 (pre-CRT RRx-001) over the other two Arms and the inferiority of Arm 3 reflect the biological longevity of RRx-001. RRx-001's inhibitory impact on SOM's pathogenesis lasted almost until the completion of the radiation course.

### Example 3. Phase 2b Randomized Trial to Assess the Safety and Efficacy of RRx-001 for the Attenuation of Severe Oral Mucositis in Patients Receiving Concomitant Chemoradiation for the Treatment of Locally Advanced Squamous Cell Carcinoma of the Oral Cavity or Oropharynx (KEVLAR)

Example 3 evaluates efficacy of two dosing regimens of RRx-001 as compared to a placebo in terms of the attenuation of severe oral mucositis (SOM, WHO Grade≥3) in patients receiving CRT for the treatment cancers of the oral cavity or oropharynx through IMRT. Example 3 also evaluates efficacy of two dosing regimens of RRx-001 as compared to the placebo in terms of the attenuation of severe oral mucositis (SOM, WHO Grade≥3) in patients receiving CRT for the treatment cancers of the oral cavity or oropharynx through 60 Gy.

### Inclusion Criteria

The inclusion criteria includes the following:
1. Patients have pathologically confirmed diagnosis of squamous cell carcinoma (SCC) of the oral cavity or oropharynx. Patients with primary cancers that are presumed to be of oropharyngeal origin may be included if they meet radiation field dosing criteria as specified in Inclusion Criterion #2 below. HPV determination is made for all patients.
2. Radiation Treatment. The patients receive standard IMRT with daily fractions of 2.0 to 2.2 Gy for a total cumulative dose of 60-72 Gy in conjunction with definitive or adjuvant chemotherapy. The planned radiation treatment fields include at least two oral sites (soft palate, floor of mouth, buccal mucosa, tongue) that are each planned to receive a total of >55 Gy. Patients who receive prior surgery are eligible, provided that they are fully recovered from surgery, and patients who may have received surgery in the future are eligible.
3. ECOG performance status≤2.
4. Participants have adequate organ and marrow function as defined below:
   i. Absolute neutrophil count (ANC)≥1,500/mm³
   ii. Platelets≥75,000/mm³
   iii. Hemoglobin≥9.0 g/dL
5. Participants have adequate renal and liver function as indicated by:
   i. Serum creatinine acceptable for treatment with cisplatin per institutional guidelines)
   ii. Total bilirubin≤1.5× upper-normal limit (ULN)
   iii. Aspartate aminotransferase (AST) and alanine aminotransferase (ALT)≤3.0×ULN
   iv. Alkaline phosphatase≤2.5×ULN
6. Human papilloma virus (HPV) status in tumor is documented using tumor immunohistochemistry for HPV-p16 or other accepted test such as in situ hybridization for patients with cancers of the oropharynx or base of tongue,
7. Age 18 years or older
8. Patient has to consent to the access, review, and analysis of previous medical and cancer history, including imaging data, by the sponsor or a third party nominated by the sponsor.
9. Participant is able and willing to understand and sign a written informed consent document.
10. Women of childbearing potential and men with partners of child-bearing potential agree to use adequate contraception (hormonal or barrier method of birth control) prior to study entry, for the duration of study participation, and for 90 days following completion of therapy. Under the criteria, a woman of child-bearing potential is any female (regardless of sexual orientation, having undergone a tubal ligation, or remaining celibate by choice) who meets the following criteria:
   i. Has not undergone a hysterectomy or bilateral oophorectomy; or
   ii. Has not been postmenopausal for at least 12 consecutive months
11. Adequate visual access to permit examination of the following oral cavity sites: lips, buccal mucosa, floor of mouth, ventral and lateral tongue, and soft palate.

### Exclusion Criteria

The exclusion criteria includes the following:
1. Prior radiotherapy to the head and neck region.
2. Prior induction chemotherapy.
3. Tumors of the lips, salivary gland, nasopharynx, hypopharynx, or larynx.
4. Patients with simultaneous primaries
5. Stage IV, MI (distant metastasis)
6. Prior or current use of approved or investigational anticancer agent other than those provided in this study.
7. Grade 3 or 4 dysphagia or odynophagia (National Cancer Institute Common Toxicity Criteria, version 5.0) or inability to eat a normal (solid) diet
8. Requirement at baseline for parenteral or gastrointestinal tube-delivered nutrition for any reason or prophylactic insertion of gastrostomy tube with dependency on tube feeding at baseline.
9. Current use of analgesics (prescription and over the counter such as pregabalin, gabapentin, skeletal muscle relaxants, benzodiazepines, sedative/hypnotics, anxiolytics, oral analgesics, NSAIDs and opioids) are prohibited.
10. Malignant tumors other than squamous cell carcinoma of the head and neck within last 5 years, unless treated definitively and with low risk of recurrence in the judgment of the treating investigator.
11. Active infectious disease excluding oral candidiasis.
12. Presence of oral mucositis (WHO Score≥Grade 1) or other oral mucosal ulceration at baseline.
13. Untreated active oral or dental infection
14. Known history of human immunodeficiency virus or active hepatitis B or C.
15. Any significant medical diseases or conditions, as assessed by the investigators and sponsor, that would substantially increase the medical risks of participating in this study (i.e., uncontrolled diabetes, NYHA II-IV congestive heart failure, myocardial infarction within 6 months of study, severe chronic pulmonary disease or active uncontrolled infection, uncontrolled or clinically relevant pulmonary edema)
16. Pregnant or nursing,
17. Known allergies or intolerance to cisplatin or other platinum-containing compounds.
18. Sjogren syndrome

### Procedure

The Phase 2b randomized clinical trial assesses the safety and efficacy of two schedules of RRx-001+CRT compared to placebo+CRT in attenuating severe oral mucositis in patients receiving CRT for locally advanced SCC of the oral cavity or oropharynx. The primary objective is to examine the efficacy of two dosing regimens of RRx-001 vs. placebo in the attenuation of SOM in patients receiving CRT for the treatment of SCC of the oral cavity or oropharynx. The secondary objective is to assess the safety and tolerability of two dosing schedules of RRx-001 during CRT treatment and for 6 to 8 weeks following the end of CRT. The long-term effect of RRx-001 on tumor response compared to standard of care CRT is also assessed.

To be eligible, the subjects are selected from a population of pathologically confirmed diagnosis of locally advanced squamous cell carcinoma of the oral cavity or oropharynx planned to be treated with IMRT plus concurrent cisplatin CRT. The Screening phase is ≤4 weeks before randomization.

Patients meeting the eligibility criteria and consenting to participate were randomized 1:1:1 to one of the two regimens of RRx-001+CRT or placebo+CRT. Approximately 216 patients (72 per Arm) are randomized into three groups, or Arms. A fourth Arm receives the CRT alone. To be evaluable, patients must complete at least 4 weeks of CRT, or have a cumulative radiation exposure of at least 60 Gy or have reached a WHO score of 3 or 4, prior to 4 weeks of CRT or prior to 60 Gy. The oral mucositis grading system by the WHO is shown below in Table 6.

TABLE 6
Oral Mucositis Grading System by the WHO

| Grade | Description |
|---|---|
| 0 (none) | None |
| I (mild) | Oral soreness, erythema |
| II (moderate) | Oral erythema, ulcers, solid diet tolerated |
| III (severe) | Oral ulcers, liquid diet only |
| IV (life-threatening) | Oral alimentation impossible |

RRx-001 treatment Arms (8 mg or 4 mg), given twice weekly during the 2 weeks prior to the start of CRT, only receive dexamethasone (10 mg intravenously or orally), or an equivalent dosage of another steroid, while the placebo group receives saline premedication. The groups or Arms were randomized as follows:
Arm 1: RRx-001 Pretreatment+CRT
   8 mg RRx-001 given twice weekly during the 2 weeks prior to the start of CRT (4 doses total). Followed by CRT.
Arm 2: RRx-001 Pretreatment+CRT
   4 mg RRx-001 given twice weekly during the 2 weeks prior to the start of CRT (4 doses total). Followed by CRT.
Arm 3 (Control): Placebo Pretreatment+CRT
   Placebo given twice weekly during the 2 weeks prior to the start of CRT. Followed by CRT. No doses of RRx-001 are administered.
Arm 4: CRT
   CRT alone. No doses of RRx-001 are administered.

RRx-001/Placebo administration occurs greater than 48 hours apart. Cisplatin is prescribed as Option 1 or Option 2, where Option 1 included 100 mg/m² every 3 weeks (Q3W) on Day 1 (±2 days) of Weeks 1, 4, and 7 and Option 2 included 40 mg/m² weekly (QW) on Day 1 (±2 days) of weeks 1, 2, 3, 4, 5, 6, and 7. At no point will cisplatin be given within 24 hours of RRx-001. IMRT consists of single daily fractions of 2.0 to 2.2 Gy with a cumulative radiation dose between 60 Gy and 72 Gy, lasting 6 to 7 weeks in duration.

The treatment phase is followed by a follow-up period. Patients are followed weekly from the last dose of IMRT until WHO oral mucositis grade≤1. Patients who do not develop SOM during the treatment period do enter the post-treatment mucositis observation follow-up and are followed until the 28-day safety visit. Long-term follow-up may be extended to 24 months following the last dose of the IMRT. Patients are followed for safety endpoints throughout the study.

### Standardized Schedule of Assessments (Pre-CRT and CRT)

The procedure for standardization of patient assessments includes the following. The patient cancer and medical history is recorded, as well as previous oral history (i.e., previous occurrences of oral mucositis, history of smoking, alcohol consumption, dry mouth (xerostomia), chewing tobacco usage, and dental history). To meet the inclusion criteria, patients must have had pathologically confirmed diagnosis of SCC of the oral cavity or oropharynx.

An oral mucositis assessment is performed by a trained on-site assessor at screening, Day 1 prior to start of Pre-CRT Treatment, prior to the start of CRT and twice a week (no less than 48 hours apart) during Combination Treatment (Weeks 1-7). Lesions are scored by a central assessor, according to the WHO scoring criteria. Each patient completes an oral mucositis weekly Questionnaire and MD Anderson Dysphagia Inventory (MDADI) at Screening and weekly during CRT (Weeks 1-7). The OMDQ is performed on the same day each week (+1 Day). Lab assessments are performed at screening, weekly during pretreatment and CRT, and as clinically indicated per institutional guidelines.

Lab assessments are performed as defined below:

### Complete Blood Count w/ Differential

White Blood Cell Count (WBC)
Red Blood Cell Count (RBC)
Hematocrit (Hct)
Hemoglobin (Hgb)
Platelets
Differential:
   Neutrophils
   Lymphocytes
   Monocytes
   Eosinophils
   Basophils

### Complete Metabolic Panel

Albumin
Blood Urea Nitrogen (BUN)
Calcium
Bicarbonate
Chloride
Creatinine
Glucose
Potassium
Sodium
Total Bilirubin
Total Protein
Alanine Aminotransferase (ALT).
Alkaline Phosphatase (ALP)
Aspartate Aminotransferase (AST)

### Pregnancy Test

Urine human chorionic gonadotropin
Serum beta-human chorionic gonadotropin

Tumor assessment is recorded at baseline and the administration of RRx-001/Placebo is performed. The HPV status using p16 is known prior to treatment and all women of childbearing potential must demonstrate a negative serum or urine pregnancy test required within 28 calendar days prior to the start of treatment.

Standardized Schedule of Follow-Up Assessments when OM Grade≤1 at End of CRT

A comprehensive physical exam (including vital signs, ECOG status, swallowing assessment, and assessment for percutaneous gastrostomy tube) is performed on Day 28. Oral mucositis assessment is performed by a trained on-site assessor who scored lesions according to WHO scoring criteria. The OMWQ and MDADI is also completed at 28-day safety follow up (Week 11), along with lab assessments. Follow-up imaging is performed prior to initiating a new therapy or at minimum every 12 weeks to assess response. Imaging continues until the 24 month follow up to assess for progression. Patients who experience adverse events related to the investigational agent RRx-001 are followed monthly until the event is resolved or is assessed as a grade≤1.

Standardized Schedule of Follow-Up Assessments when OM Grade≥2 at End of CRT

The mucositis observation follow-up Day 1 is the day following the last dose of IMRT. A comprehensive physical exam is performed at week 11 (28d post-IMRT) and includes the following: ECOG status, swallowing assessment, and assessment for percutaneous gastrostomy tube. If the subject achieves OM grade≤1 prior to week 11, week 11 (28d post IMRT) is performed. An ECOG status by a radiation oncologist and/or medical oncologist is performed weekly with OMWQ and MDADI during the mucositis observation follow-up until OM grade≤1 is achieved. Lesions are scored by a central assessor according to WHO scoring criteria. Tobacco and alcohol use is recorded as part of the medical/social history. Lab assessments are performed at week 11 (28d post-IMRT), and as clinically indicated per institutional guidelines. Follow-up imaging is performed per institutional standards, prior to initiating a new therapy or at minimum every 12 weeks to assess response. Patients who experience adverse events (AEs) related to the investigational agent RRx-001, are followed monthly until the AE is resolved or is assessed as a grade≤1.

### Primary Efficacy Endpoints

The primary efficacy endpoint is the incidence of SOM defined as the proportion of patients with any WHO Grade≥3 (severe to life threatening) oral mucositis during the observation period from the start of CRT through 60 Gy. The primary efficacy analysis of SOM is based on a modified ITT (mITT) population. The mITT population is defined as excluding those trial participants in the intention-to-treat (ITT) population that did not receive the intended study interventions despite being assigned to an intervention. The null hypothesis of equality of SOM incidence between all three treatment Arms are tested against the alternative that at least one of the RRx-001 treatment Arms differed from the placebo.

### Secondary Efficacy Endpoints

Secondary efficacy endpoints include: duration of SOM (through the last day of radiation therapy, DoSOM), time onset to SOM (ttSOM) (defined as the time interval measured from the start of the observation period to the first time SOM is observed), incidence and severity of dysphagia, narcotic use through resolution of SOM, cumulative radiation dose to onset of SOM compared between RRx-001 Arms and placebo, and incidence of Grade 4 OM through 60 Gy.

### Assessment of Safety and AEs

Safety endpoints include an incidence AEs, utilizing National Cancer Institute Common Terminology for AEs (Version 5.0), treatment emergent adverse event (TEAE), and serious TEAE reporting instruments, as well as tumor response evaluation at 6- and-12 months post treatment for rates of locoregional control, including progression-free survival.

An AE is any untoward medical occurrence in a subject or clinical investigation subject administered a pharmaceutical product and that does not necessarily have a causal relationship with this treatment. All AEs are assessed by an investigator and recorded. The reported verbatim term is documented including the date of onset and resolution, NCI-CTCAE grade of severity, relationship to study medication, outcome, and action taken.

The AEs are reported starting immediately after the subject receives the first dose of RRx-001 or placebo through 28 days after the last dose of the investigational product, week 12 if OM score of ≥1 or resolution of adverse events attributable to RRx-001. An adverse reaction (AR) refers to any AE caused by a drug. An AR or adverse drug reaction (ADR) is a subset of all suspected adverse reactions (SARs) for which there was reason to conclude that the drug caused the event. Adverse reactions are a subset of all suspected AEs for which there was reason to conclude that the drug caused the AE. An AE or Suspected adverse reaction is considered “unexpected” if it is not listed in the Investigator Brochure (IB) or if it is not listed at the specificity or severity that has been observed.

An AE are considered “serious” if it results in any of the following outcomes:
Death;
Life-threatening (subject was at immediate risk of death);
Inpatient hospitalization (of any length) or prolongation of existing hospitalization.
Resulted in persistent or significant incapacity or substantial disruption of the ability to conduct normal life functions.
Congenital anomaly/birth defect in the offspring of a subject who received study medication.
Other: Important medical events that may not result in death, be immediately life-threatening, or require hospitalization, were considered a SAE when, based upon appropriate medical judgment, they potentially jeopardized the subject and may have required medical or surgical intervention to prevent one of the outcomes listed in this definition, examples of such events included: intensive treatment in an emergency room or at home for allergic response, blood dyscrasias or convulsions that did not result in hospitalization, development of drug dependency or drug abuse, etc.

In cases where the AE is not resolved up to 28 days after the last dose of study drug, the final outcome of the ongoing and new unrelated AEs are captured as “Not Recovered/Not Resolved” or “Recovering/Resolving” whichever was applicable. AEs attributable to RRx-001 are monitored through resolution.

### Example 4. Effect of RRx-001 on Halo Toxicities of Oral Mucositis in Patients Treated with Concomitant Chemoradiation for Locally Advanced Head and Neck Cancer

The results of Example 2, the open-labeled Phase 2a trial (PREVLAR; NCK03515538), suggested that infusion of RRx-001 attenuated the course and severity of severe oral mucositis in patients being treated with concomitant chemoradiation (cisplatin/IMRT) for cancers of the mouth or oropharynx without impeding tumor response. Given its mechanism of action, Example 4 investigated the potential “halo effects” of RRx-001 on other regimen-related toxicities.

Treatment regimens of radiotherapy with CRT represent the current standard of care for patients with locoregional HNSCC. Despite radiation-sparing techniques, CRT is associated with a range of complications as a consequence of the radiation fields and the systemic effects of treatment, which include mucositis, salivary gland dysfunction, skin injury, and toxicities affecting the marrow, kidneys, and neurological function.

The results of Example 2 suggested that infusion of RRx-001 safely attenuated the course and severity of severe OM without impeding tumor response. Given the shared pathobiology of mucositis with other tissue-based radiation-associated toxicities and the systemic exposure of RRx-001, a comparison of the incidence of AEs as reported is informative relative to potential halo effects of RRx-001.

Two-sided Fisher's exact test comparing the related AEs incidences between RRx-001 Arms and SOC (control) (see Table 7) demonstrated statistical significance (p<0.05) unaltered by the multiplicity of comparisons between test and control Arms for several AEs.

TABLE 7
Baseline Patient Characteristics (*mITT population)

| | Safety Population Demographics | | | | |
|---|---|---|---|---|---|
| | No. (%) of Patients | | | | |
| | | Arm 2: RRX-001 | Arm 3: RRX-001 | | |
| | | Pretreatment + 2 | Pretreatment + 6 | | |
| | Arm 1: RRX-001 | Concurrent | Concurrent | | |
| | Pretreatment + SOC | Doses + SOC | Doses + SOC | Arm 4: | |
| | Chemoradiation | Chemoradiation | Chemoradiation | Chemoradiation | All Patients |
| Characteristic | (n = 12) | (n = 11) | (n = 13) | Only (n = 10) | (n = 46) |

Sex

| | | | | | |
|---|---|---|---|---|---|
| Male | 10 (83.3) | 9 (81.8) | 12 (92.3) | 9 (90) | 40 (87) |
| Female | 2 (16.3) | 2 (18.2) | 1 (7.7) | 1 (10) | 6 (13) |
| Median Age, years | 59 | 58 | 61 | 60 | 60 |

Race

| | | | | | |
|---|---|---|---|---|---|
| Asian | 0 | 0 | 1 (7.7) | 1 (10) | 2 (4.3) |
| Black or African American | 1 (8.3) | 1 (9.1) | 0 | 1 (10) | 3 (6.5) |
| White | 11 (91.7) | 10 (90.9) | 12 (92.3) | 8 (80) | 41 (89.1) |
| Hispanic or Latino | 0 | 1 (9.1) | 0 | 1 (10) | 2 (4.3) |
| Non-Hispanic | 11 (91.7) | 10 (90.9) | 13 (100) | 9 (90) | 43 (93.5) |

HPV Status

| | | | | | |
|---|---|---|---|---|---|
| Unknown | 0 | 0 | 0 | 0 | 0 |
| Negative | 2 (16.7) | 2 (18.2) | 2 (15.4) | 0 | 6 (13) |
| Not Done | 0 | 0 | 2 (15.4) | 1 (10) | 3 (6.5) |
| Positive | 10 (83.3) | 9 (81.8) | 9 (69.2) | 9 (90) | 37 (80.4) |

Tobacco Use History

| | | | | | |
|---|---|---|---|---|---|
| No (never) | 7 (58.3) | 7 (63.6) | 6 (46.2) | 6 (60) | 26 (56.5) |
| Yes | 5 (41.7) | 4 (36.4) | 7 (53.8) | 4 (40) | 20 (43.5) |

Alcohol Use History

| | | | | | |
|---|---|---|---|---|---|
| Missing | 4 (33.3) | 4 (36.4) | 2 (15.4) | 3 (30) | 13 (28.3) |
| Current User | 5 (41.7) | 4 (36.4) | 8 (61.5) | 6 (60) | 23 (50) |
| Former User | 3 (25) | 3 (27.3) | 3 (23.1) | 1 (10) | 10 (21.7) |

History of Smokeless Tobacco

| | | | | | |
|---|---|---|---|---|---|
| Missing | 7 (58.3) | 7 (63.6) | 6 (46.2) | 6 (60) | 26 (56.5) |
| Former User | 0 | 0 | 2 (15.4) | 1 (10) | 3 (6.5) |
| Never User | 5 (41.7) | 4 (36.4) | 5 (38.5) | 3 (30) | 17 (37) |

History of Cigar Smoking

| | | | | | |
|---|---|---|---|---|---|
| Missing | 7 (58.3) | 7 (63.6) | 6 (46.2) | 7 (70) | 27 (58.7) |
| Former Smoker | 1 (8.3) | 0 | 2 (15.4) | 0 | 3 (6.5) |
| Never Smoker | 4 (33.3) | 4 (36.4) | 5 (38.5) | 3 (30) | 16 (34.8) |

Surgical Status

| | | | | | |
|---|---|---|---|---|---|
| Post-Operative | 3 (25) | 3 (27.3) | 4 (30.8) | 1 (10) | 11 (23.9) |
| Definitive (no prior surgery) | 9 (75) | 8 (72.7) | 9 (69.2) | 9 (90) | 35 (76.1) |

Cisplatin Schedule*

| | | | | | |
|---|---|---|---|---|---|
| Weekly | 6 (50) | 6 (60) | 10 (76.9) | 10 (90.9) | 32 (69.6) |
| Triweekly | 6 (50) | 4 (40) | 3 (23.1) | 1 (9.1) | 14 (30.4) |

Primary Tumor Site

| | | | | | |
|---|---|---|---|---|---|
| Oropharyngeal | 10 (83.3) | 10 (90.9) | 11 (84.6) | 9 (90) | 40 (87) |
| Oral Cavity | 2 (16.7) | 1 (9.1) | 2 (15.4) | 1 (10) | 6 (13) |

Toxicities associated with presumed salivary gland injury were consistently higher in the control patients compared to RRx-001 treated individuals. Compared to control patients, patients in Arms 1 and 2 had a lower incidence of dry mouth (none vs. 60%, p=0.0028), dysphagia (none vs. 70%, p=0.0007), and salivary duct inflammation (none vs. 30%; N.S.). Similarly, radiation-induced skin injury was blunted in RRx-001 patients (Arm 1 none, Arm 2 18.2%, control 70%, p=0.0007 Arm 1 compared to the control). Oral pain was less in RRx-001-treated patients (Arm 1 0%, Arm 2 9.1%, control 50%, Fisher's test p=0.0095 Arm 1 compared to the control), which likely impacted weight loss (none vs. 50%, p=0.0095). Related anemia (Arm 1 0%, Arm 2 0%, control 30%), related constipation (Arm 1 0%, Arm 2 0%, control 50%, Fisher's test p=0.0095 Arm 1 vs control), related oral dysesthesia (Arm 1 0%, Arm 2 0%, control 30%), related vomiting (Arm 1 1%, Arm 2 1%, control 60%, Fisher's test p=0.0028), and neutrophil count decrease (Arm 1 0%, Arm 2 0%, control 30%). RRx-001 treatment did not statistically impact marrow-related AEs (anemia, neutropenia), renal function or the incidence of candidiasis.

Squamous cell cancers of the oral cavity, oropharynx, larynx, or hypopharynx, are the seventh most common cancers worldwide. Concomitant chemoradiation is the mainstay of treatment for patients with locally advanced disease (Stages III to IVB). An inevitable byproduct of standard of care treatment with chemoradiation is collateral damage to normal tissues, which results in a cluster of acute toxicitysphagia, and impaired function of the salivary glands. Not only do these toxicities impact patients' tolerance of optimal cancer treatment, but some also predispose to chronic changes that lead to the risk of additional disease post-cancer therapy.

RRx-001 is a small molecule direct NLRP3 inhibitor and Nrf2 activator with anti-cancer activity, anti-infective activity, and normal tissue protective properties against the toxicities of chemotherapy and radiation, including severe oral mucositis. See K. J. Jurgensen, et al., (2021) Front Pharmacol. 12:676396; A. Tichy, et al. (2022) Front Pharmacol. 13:983702; and M. Bonomi, et al. (2023) Int J Radiat Oncol Biol Phys. S0360-3016 (22) 03683-5. Unique to RRx-001's mucositis prevention activity, was the observation that the drug was effective when administered over two weeks prior to the start of CRT. Subsequent infusions either marginally improved this observation or had no effect at all. Toxicity attributable specifically to RRx-001 was localized, mild, and transient, resolved immediately with cessation of RRx-001 infusion, and did not compromise treatment with RRx-001 or chemoradiation. These localized infusion-specific effects are related to RRx-001-mediated nitric oxide release, as previously reported. See S. Caroen, et al. (2022) Int J Med Sci. 19(11):1628-1630; and V. P. Jani, et al. (2021) Int J Mol Sci. 22(9):4713.

It has been well-established that toxicities associated with cancer therapy typically occur in clusters. It is rare for patients to develop only one side effect. In the case of concomitant chemoradiation for head and neck cancers, in addition to mucositis, patients are prone to damage to the parotid glands and ducts and radiation-induced dermatitis with consequent xerostomia, dysphagia, weight loss, and increased risk of candidiasis. As shown by this Example, given its systemic route of administration, RRx-001 favorably impacted other toxicities associated with mucositis. RRx-001 was most active as an anti-mucositis agent when administered prior to CRT (Arm 1) or prior to CRT with additional dosing on weeks 2 and 5 (Arm 2), compared to Arm 3 or control, and these two dosing schedules also appeared to significantly reduce some of the serious sequelae secondary to radiation therapy including dry mouth (xerostomia), dysphagia, skin injury, salivary duct inflammation, and weight loss (see Table 7).

TABLE 7
Comparison of reported adverse events using Common Terminology Criteria
for Adverse Events (CTCAEv.4) criteria between RRx-001 and control Arms

| | | Arm 2: RRx-001 | Arm 3: RRx-001 | | |
|---|---|---|---|---|---|
| | Arm 1: RRx-001 | Pre-treatment + | Pre-treatment + | | |
| | Pre-treatment + | 2 Concurrent | 6 Concurrent | Arm 4: SOC | |
| | SOC | Doses + SOC | Doses + SOC | Chemoradiation | |
| | Chemoradiation | Chemoradiation | Chemoradiation | Only (Control) | Total |
| AE | (N = 12) | (N = 11) | (N = 13) | (N = 10) | (N = 46) |
| MARROW | | | | | |
| Anemia | 0 (0) | 0 (0) | 4 (30.8) | 3 (30) | 7 (15.2) |
| Neutrophil count | 0 (0) | 0 (0) | 1 (7.7) | 3 (30) | 4 (8.7) |
| decreased | | | | | |
| Platelet count | 0 (0) | 0 (0) | 3 (23.1) | 2 (20) | 5 (10.9) |
| decreased | | | | | |
| White blood cell | 0 (0) | 0 (0) | 1 (7.7) | 3 (30) | 4 (8.7) |
| count decreased | | | | | |
| TISSUE | | | | | |
| Dry mouth | 0 (0) | 0 (0) | 4 (30.8) | 6 (60) | 10 (21.7) |
| Dysphagia | 0 (0) | 0 (0) | 2 (15.4) | 7 (70) | 9 (19.6) |
| Salivary duct | 0 (0) | 0 (0) | 1 (7.7) | 3 (30) | 4 (8.7) |
| inflammation | | | | | |
| Radiation skin | 0 (0) | 2 (18.2) | 4 (30.8) | 7 (70) | 13 (28.3) |
| injury | | | | | |
| Laryngeal | 0 (0) | 0 (0) | 0 (0) | 2 (20) | 2 (4.3) |
| inflammation | | | | | |
| Dermatitis | 0 (0) | 0 (0) | 0 (0) | 1 (10) | 1 (2.2) |
| INFECTION | | | | | |
| Candida infection | 0 (0) | 1 (9.1) | 3 (23.1) | 4 (40) | 8 (17.4) |
| Oral candidiasis | 1 (8.3) | 0 (0) | 1 (7.7) | 1 (10) | 3 (6.5) |
| RENAL | | | | | |
| Blood creatinine | 0 (0) | 2 (18.2) | 0 (0) | 2 (20) | 4 (8.7) |
| increased | | | | | |
| Blood urea | 0 (0) | 0 (0) | 0 (0) | 2 (20) | 2 (4.3) |
| increased | | | | | |
| WEIGHT/DIET | | | | | |
| Weight decreased | 0 (0) | 0 (0) | 2 (15.4) | 5 (50) | 7 (15.2) |
| Decreased | 0 (0) | 0 (0) | 4 (30.8) | 2 (20) | 6 (13) |
| appetite | | | | | |
| SENSORY | | | | | |
| Glossodynia | 0 (0) | 0 (0) | 0 (0) | 1 (10) | 1 (2.2) |
| Oral dysaesthesia | 0 (0) | 0 (0) | 0 (0) | 3 (30) | 3 (6.5) |
| Oral dysesthesia | 0 (0) | 0 (0) | 0 (0) | 1 (10) | 1 (2.2) |
| Dysgeusia | 0 (0) | 0 (0) | 7 (53.8) | 9 (90) | 16 (34.8) |
| Oral pain | 0 (0) | 1 (9.1) | 4 (30.8) | 5 (50) | 10 (21.7) |
| OTHER | | | | | |
| Constipation | 0 (0) | 0 (0) | 0 (0) | 5 (50) | 5 (10.9) |
| Vomiting | 1 (8.3) | 1 (9.1) | 3 (23.1) | 6 (60) | 11 (23.9) |

CTCAEv.4 are a set of criteria for the standardized classification of AEs of drugs used in cancer therapy.

Such selective cytoprotection decreased the main acute toxicities of CRT, such as mucositis, infection, xerostomia, and dysphagia, as well as late side effects, such as xerostomia, loss of taste, dysphagia, and fibrosis. The improvement of xerostomia is significant since dry mouth can lead to dysphagia, dysgeusia, oral pain, dental caries, oral infection, periodontal disease, and malnutrition both in the short and long term.

Many variations may be made to the embodiments described herein. All variations, including combinations of embodiments, are intended to be included within the scope of this disclosure. The description of the embodiments herein can be practiced in many ways. Any terminology used herein should not be construed as restricting the features or aspects of the disclosed subject matter. The scope should instead be construed in accordance with the appended claims.

What is claimed is:

1. A method for treating or preventing normal tissue injury from at least one of radiation and chemotherapy in a subject in need thereof, the method comprising:
administering an effective amount of RRx-001, or a pharmaceutically acceptable salt thereof, to the subject.

2. The method of claim 1, wherein the effective amount of the RRx-001, or a pharmaceutically acceptable salt thereof, is a therapeutically effective amount of the RRx-001, or a pharmaceutically acceptable salt thereof.

3. The method of claim 2, wherein the therapeutically effective amount of the RRx-001, or a pharmaceutically acceptable salt thereof, is in a range of about 0.1 mg to about 500 mg.

4. The method of claim 3, wherein the therapeutically effective amount of the RRx-001, or a pharmaceutically acceptable salt thereof, is in a range of about 0.5 mg to about 200 mg.

5. The method of claim 3, wherein the therapeutically effective amount of the RRx-001, or a pharmaceutically acceptable salt thereof, is in a range of about 5 mg to about 50 mg.

6. The method of claim 3, wherein the therapeutically effective amount of the RRx-001, or a pharmaceutically acceptable salt thereof, is in a range of about 10 mg to about 30 mg.

7. The method of claim 1, wherein the radiation comprises ionizing radiation.

8. The method of claim 7, wherein the normal tissue injury results from ionizing radiation.

9. The method of claim 1, wherein the normal tissue injury results from the chemotherapy.

10. The method of any one of claims 1-9, wherein the method treats the normal tissue injury from the at least one of the radiation and the chemotherapy in the subject in need thereof.

11. The method of any one of claims 1-9, wherein the method prevents the normal tissue injury from the at least one of the radiation and the chemotherapy in the subject in need thereof.

12. The method of any one of claims 1-11, wherein the subject has a locally advanced solid tumor.

13. The method of claim 12, wherein the locally advanced solid tumor is selected from the group consisting of: a gastrointestinal malignancy, rectal cancer, anal cancer, head and neck cancer, gynecological cancer, breast cancer, hepatocellular cancer, esophageal cancer, lung cancer, genitourinary cancer, gastrointestinal tract cancer, genitourinary cancer, hepatocellular carcinoma, glioblastoma, and sarcoma.

14. The method of any one of claims 1-13, wherein the normal tissue injury comprises a condition that is selected from the group consisting of: mucositis, dysphagia, dyspepsia, laryngeal inflammation, oral dysesthesia, vomiting, salivary duct inflammation, esophagitis, any gastrointestinal distress, myelosuppression, impotence, infertility, dermatitis, hair loss, and increased creatinine.

15. The method of any one of claims 1-13, wherein the normal tissue injury comprises secondary cancer development.

16. The method of any one of claims 1-13, wherein the normal tissue is selectively protected from injury relative to tumor tissue in the subject.

17. The method of any one of claims 1-16, wherein the subject has a genetic syndrome that predisposes the subject to head and neck cancer.

18. The method of claim 17, wherein the genetic syndrome is selected from the group consisting of; Fanconi's anemia, Xeroderma pigmentosum, Ataxia telangiectasia, Li Fraumeni Syndrome, Retinoblastoma, Dyskeratosis congenita, Bloom's Syndrome, and Rothmund-Thompson.

19. The method of any one of claims 1-13, wherein the subject has a head and neck cancer attributable to one or more of cigarette smoking, alcohol drinking, infection with human papilloma virus (HPV), or Epstein-Barr virus (EBV).

20. The method of any one of claims 1-19, wherein the subject is a mammal subject.

21. The method of claim 20, wherein the mammal subject is a human subject.

22. The method of claim 20, wherein the mammal subject is an animal subject.

23. The method of any one of claims 1-22, wherein administering the effective amount of RRx-001, or a pharmaceutically acceptable salt thereof, occurs via oral administration, transdermal administration, administration by inhalation, nasal administration, topical administration, intra-aural administration, rectal administration, intravenous administration, intramuscular administration, subcutaneous administration, or intraperitoneal administration, or combinations thereof.

24. The method of claim 23, wherein administering the effective amount of RRx-001, or a pharmaceutically acceptable salt thereof, occurs via intravenous administration.

25. The method of claim 23, wherein administering the effective amount of RRx-001, or a pharmaceutically acceptable salt thereof, occurs via oral administration, and wherein the oral administration comprises a swish and spit administration.

26. The method of claim 23, wherein administering the effective amount of RRx-001, or a pharmaceutically acceptable salt thereof, occurs via oral administration, and wherein the oral administration comprises a swish and swallow administration.

27. The method of any of claims 1-22, wherein administering the effective amount of RRx-001, or a pharmaceutically acceptable salt thereof, occurs via direct intratumoral injection.

28. The method of any one of claims 2-27, wherein administering the therapeutically effective amount of RRx-001, or a pharmaceutically acceptable salt thereof, is performed via a single administration.

29. The method of any one of claims 2-27, wherein administering the therapeutically effective amount of RRx-001, or a pharmaceutically acceptable salt thereof, is performed via at least two administrations during a time period.

30. The method of claim 29, wherein the time period is up to six weeks.

31. The method of claim 29, wherein administering the therapeutically effective amount of RRx-001, or a pharmaceutically acceptable salt thereof, is performed at a frequency of between about 1 time per hour and about 1 time per month during the time period.

32. The method of claim 29, wherein administering the therapeutically effective amount of RRx-001, or a pharmaceutically acceptable salt thereof, is performed at a frequency of between about 4 times per day and about 1 time per week during the time period.

33. The method of claim 29, wherein administering the therapeutically effective amount of RRx-001, or a pharmaceutically acceptable salt thereof, is performed at a frequency of between about 2 times per day and about 3 times per week during the time period.

34. The method of claim 29, wherein administering the therapeutically effective amount of RRx-001, or a pharmaceutically acceptable salt thereof, is performed at a frequency of about one time a day during the time period.

35. The method of claim 29, wherein administering the therapeutically effective amount of RRx-001, or a pharmaceutically acceptable salt thereof, is performed at a frequency of between about 1 time per day and about 2 times per week during the time period.

36. The method of any one of claims 1-27, wherein administering the effective amount of the RRx-001, or a pharmaceutically acceptable salt thereof, comprises administering a pretreatment dose of the RRx-001, or a pharmaceutically acceptable salt thereof, at a frequency during a time period prior to another treatment.

37. The method of claim 36, wherein the other treatment comprises at least one of radiation, chemotherapy, and immunotherapy.

38. The method of claim 37, wherein the other treatment comprises the radiation, and wherein the radiation comprises ionizing radiation.

39. The method of claim 37, wherein the other treatment comprises at least one of the chemotherapy and the immunotherapy, and wherein the at least one of the chemotherapy and the immunotherapy comprises an agent selected from the group consisting of: Dexamethasone, Erbitux (Cetuximab), Avasopasem (GC4419), Pembrolizumab, Nivolumab, Hydrocortisone, Prednisone, Cyclophosphamide, Methotrexate, Paclitaxel, Carboplatin, Etoposide, Gemcitabine, Cisplatin, Oxaliplatin, Chlorambucil, Mechloethamine, Melphalan, Tocilizumab, Brentuximab Vedotin, Doxorubicin, Afatinib, Everolimus, Netupitant, Palonosetron, Imiquimod, and Fluorouracil.

40. The method of claim 39, wherein an amount of the agent is in a range of about 1 mg to about 50 mg.

41. The method of claim 39, wherein the amount of the agent is in the range of about 1 mg to about 15 mg.

42. The method of claim 36, wherein the time period is in a range of about 1 day to about 6 months.

43. The method of claim 36, wherein the time period is in a range of about 1 week to about 4 weeks.

44. The method of claim 43, wherein the time period is about 2 weeks.

45. The method of claim 36, wherein an amount of the pretreatment dose of the RRx-001, or a pharmaceutically acceptable salt thereof, is in a range of about 1 mg to about 200 mg.

46. The method of claim 45, wherein an amount of the pretreatment dose of the RRx-001, or a pharmaceutically acceptable salt thereof, is in a range of about 2 mg to about 20 mg.

47. The method of claim 36, wherein the frequency is about 1 time per week, 1 time per month, 2-5 times per week, 2-4 times per month, one time per day, two times per day, three times per day, or four times per day.

48. The method of any one of claims 36-47, further comprising:
administering at least one additional dose of the RRx-001, or a pharmaceutically acceptable salt thereof, concurrently with the other treatment.

49. The method of any one of claims 36-48, further comprising:
administering at least one additional dose of the RRx-001, or a pharmaceutically acceptable salt thereof, subsequent the other treatment.

50. The method of any one of claims 1-49, wherein the RRx-001, or a pharmaceutically acceptable salt thereof, is not administered concurrently with another treatment.

51. The method of any of claims 2-49, wherein the therapeutically effective amount of the RRx-001, or a pharmaceutically acceptable salt thereof, is administered as a composition comprising a blood product.

52. The method of claim 51, wherein the blood product comprises erythrocyte cells.

53. The method of claim 52, wherein the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis.

54. The method of claim 51, wherein the blood product is a mixture of packed red blood cells.

55. The method of claim 51, wherein the blood product is whole blood.

56. The method of claim 55, wherein the whole blood is autologous whole blood or donor-matched allogenic whole blood.

57. The method of any of claims 1-56, further comprising:
administering an agent prior to, concurrently with, or subsequent administering the effective amount of RRx-001, or a pharmaceutically acceptable salt thereof.

58. The method of claim 57, wherein administering the agent occurs during a time period in a range of about 1 week to about 6 weeks prior to administering the effective amount of RRx-001, or a pharmaceutically acceptable salt thereof.

59. The method of any one of claims 57-58, wherein the agent comprises a corticosteroid.

60. The method of claim 59, wherein the corticosteroid comprises dexamethasone.

61. The method of claim 60, wherein an amount of the agent is in a range of about 0.1 mg to about 50 mg.

62. The method of any one of claims 57-58, wherein the agent is selected from the group consisting of: a PARP inhibitor, a tyrosine kinase inhibitor, a thiol-based chemoradioprotectant agent, an EGFR inhibitor, coconut oil, MucoLox mouthwash, checkpoint inhibitors, TGF-beta, superoxide dismutase mimetics, antimicrobials, herbal supplements, defensin-mimetics, cryotherapy, an HDAC inhibitor, a DNA methyltransferase inhibitor, 5-FU, imatinib, hydroxyurea, taxol, an oncolytic virus, a checkpoint inhibitor, and a topoisomerase inhibitor.

63. The method of claim 62, wherein the agent comprises the thiol-based chemoradioprotectant agent, and wherein the thiol-based chemoradioprotectant agent is selected from the group consisting of: N-acetyl cysteine (NAC), amifostine, sodium thiosulfate (STS), D-methionine, GSH ethyl ester, and GlyNAC (Glycine and N-Acetylcysteine).

64. The method of claim 62, wherein the agent comprises the topoisomerase inhibitor, and wherein the topoisomerase inhibitor is selected from the group consisting of: irinotecan and doxorubicin.

65. The method of any one of claims 57-58, wherein the agent treats oral mucositis.

66. The method of claim 65, wherein the agent that treats oral mucositis is selected from the group consisting of: GM-CSF, palifermin, pilocarpine, gabapentin, keratinocyte growth factor (KGF), coconut oil, MucoLox, cetuximab, a checkpoint inhibitor, transforming growth factor beta (TGF-β), an epidermal growth factor, honey, benzydamine, a corticosteroid, rebamipide, an antimicrobial agent, misoprostol, Amifostine, brilacidin, L-glutamine, low-level laser therapy, laser and light therapy, cryotherapy, vitamin E, pentoxyfyilline, GC4419, clonidine, melatonin, a probiotic, actovegin, aloe vera, allopurinol, azithromycin, Black Mulberry Molasses, Glycerin payayor, Qingre Liyan decoction, erythropoietin mouthwash, EC-18, sucralfate, Avasopasem manganese, and lidocaine, wherein the checkpoint inhibitor is selected from the group consisting of: pembrolizumab and nivolumab, and wherein the antimicrobial agent is selected from the group consisting of: chlorhexidine, polymyxin E, and amphotericin.

67. The method of any one of claims 57-66, wherein administering the agent prior to, concurrently with, or subsequent administering the effective amount of RRx-001, or a pharmaceutically acceptable salt thereof, prevents at least one side effect associated with the agent.

68. The method of any one of claims 57-67, wherein administering the agent prior to, concurrently with, or subsequent administering the effective amount of RRx-001, or a pharmaceutically acceptable salt thereof, reduces at least one side effect associated with the agent.

69. The method of any one of claims 1-68, wherein administering the effective amount of the RRx-001, or a pharmaceutically acceptable salt thereof, comprises administering a pretreatment dose of the RRx-001, or a pharmaceutically acceptable salt thereof, at a frequency during a time period prior to another treatment.

70. The method of claim 69, wherein the other treatment comprises at least one of radiation, chemotherapy, and immunotherapy.

71. The method of claim 70, wherein the other treatment comprises the radiation, and wherein the radiation comprises ionizing radiation.

72. The method of claim 70, wherein the other treatment comprises the at least one of the chemotherapy and the immunotherapy, and wherein the at least one of the chemotherapy and the immunotherapy comprises an agent selected from the group consisting of: Dexamethasone, Erbitux (Cetuximab), Avasopasem (GC4419), Pembrolizumab, Nivolumab, Hydrocortisone, Prednisone, Cyclophosphamide, Methotrexate, Paclitaxel, Carboplatin, Etoposide, Gemcitabine, Cisplatin, Oxaliplatin, Chlorambucil, Mechloethamine, Melphalan, Tocilizumab, Brentuximab Vedotin, Doxorubicin, Afatinib, Everolimus, Netupitant, Palonosetron, Imiquimod, and Fluorouracil.

73. The method of claim 72, wherein an amount of the agent is in a range of about 1 mg to about 50 mg.

74. The method of claim 73, wherein the amount of the agent is in the range of about 1 mg to about 15 mg.

75. The method of any one of claims 69-74, wherein the time period is in a range of about 1 day to about 6 months.

76. The method of claim 75, wherein the time period is in a range of about 1 week to about 4 weeks.

77. The method of claim 76, wherein the time period is about 2 weeks.

78. The method of any one of claims 69-77, wherein an amount of the pretreatment dose of the RRx-001, or a pharmaceutically acceptable salt thereof, is in a range of about 1 mg to about 200 mg.

79. The method of any one of claims 69-77, wherein an amount of the pretreatment dose of the RRx-001, or a pharmaceutically acceptable salt thereof, is in a range of about 2 mg to about 20 mg.

80. The method of any one of claims 69-79, wherein the frequency is about 1 time per week, 1 time per month, 2-5 times per week, 2-4 times per month, one time per day, two times per day, three times per day, or four times per day.

81. The method of any one of claims 2-6, 10-35, and 51-56, wherein administering the therapeutically effective amount of the RRx-001, or a pharmaceutically acceptable salt thereof, consists of: administering a pretreatment dose of the RRx-001, or a pharmaceutically acceptable salt thereof.

82. The method of claim 81, wherein an amount of the pretreatment dose of the RRx-001, or a pharmaceutically acceptable salt thereof, is in a range of about 1 mg to about 200 mg.

83. The method of claim 82, wherein an amount of the pretreatment dose of the RRx-001, or a pharmaceutically acceptable salt thereof, is in a range of about 2 mg to about 20 mg.

84. The method of any one of claims 1-83, wherein administering the effective amount of the RRx-001, or a pharmaceutically acceptable salt thereof, increases tumor ablation in the subject.

85. The method of any one of claims 1-84, wherein administering the effective amount of the RRx-001, or a pharmaceutically acceptable salt thereof, attenuates secondary cancer development in the subject.

86. The method of any one of claims 1-85, wherein administering the effective amount of the RRx-001, or a pharmaceutically acceptable salt thereof, selectively protects normal tissue from an injury relative to tumor tissue in the subject, and wherein the injury is selected from the group consisting of: mucositis, dysphagia, dyspepsia, laryngeal inflammation, oral dysesthesia, vomiting, salivary duct inflammation, esophagitis, any gastrointestinal distress, myclosuppression, impotence, infertility, dermatitis, hair loss and increased creatinine.

87. The method of any one of claims 1-86, wherein administering the effective amount of the RRx-001, or a pharmaceutically acceptable salt thereof, results in the subject experiencing a decrease in malnutrition.

88. The method of any one of claims 1-87, wherein administering the effective amount of the RRx-001, or a pharmaceutically acceptable salt thereof, results in the subject having a lower incidence of gastrostomy-tube placement.

89. The method of any one of claims 1-88, wherein administering the effective amount of the RRx-001, or a pharmaceutically acceptable salt thereof, results in the subject having a lower incidence of weight loss.

90. The method of any one of claims 1-89, wherein administering the effective amount of the RRx-001, or a pharmaceutically acceptable salt thereof, results in a decrease in other toxicities associated with mucositis.

91. The method of any one of claims 1-21 and 23-90, wherein the subject comprises the human subject, and wherein administering the effective amount of the RRx-001, or a pharmaceutically acceptable salt thereof, increases a tolerated dose of the at least one of the radiation and the chemotherapy in treating the human subject for cancer.

92. The method of claim 91, wherein the chemotherapy is platinum-based chemotherapy.

93. The method of claim 92, wherein the platinum-based chemotherapy is selected from the group consisting of: cisplatin, oxaliplatin, carboplatin, nedaplatin, heptaplatin, and lobaplatin.

94. The method of any one of claims 91-93, wherein increasing the tolerated dose of the at least one of the radiation and the chemotherapy in treating the human subject for cancer comprises increasing a previously poorly tolerated dose of the at least one of the radiation and the chemotherapy.

95. The method of any one of claims 91-93, wherein an increased tolerated amount or relative dose intensity (RDI) of the at least one of the radiation and the chemotherapy increases subsequent to administering the effective amount of the RRx-001, or a pharmaceutically acceptable salt thereof.

96. The method of claim 94, wherein the previously poorly tolerated dose of the at least one of the radiation and the chemotherapy becomes tolerable subsequent to administering the effective amount of the RRx-001, or a pharmaceutically acceptable salt thereof.

97. The method of claim 94, wherein the increased tolerated amount of the at least one of the radiation and the chemotherapy comprises administering an increased dose of at least one of cisplatin and radiotherapy each time the at least one of the cisplatin and the radiotherapy are administered.

98. The method of claim 97, wherein the increased tolerated amount of the cisplatin each time the cisplatin is administered is in a range of about 50 mg to about 150 mg.

99. The method of any one of claims 97-98, wherein administering the effective amount of the RRx-001, or a pharmaceutically acceptable salt thereof, results in an increased anti-tumor efficacy of the at least one of the cisplatin and the radiotherapy in a treatment for cancer.

100. A composition for treating or preventing normal tissue injury from at least one of radiation and chemotherapy in a subject in need thereof, the composition comprising: an effective amount of RRx-001 or a pharmaceutically acceptable salt thereof, a blood product, and at least one agent.

101. The composition of claim 100, wherein the effective amount is a therapeutically effective amount of RRx-001 or a pharmaceutically acceptable salt thereof.

102. The composition of claim 101, wherein the therapeutically effective amount of the RRx-001, or a pharmaceutically acceptable salt thereof, is in a range of about 0.1 mg and about 500 mg.

103. The composition of claim 101, wherein the therapeutically effective amount of the RRx-001, or a pharmaceutically acceptable salt thereof, is in a range of about 0.5 mg and about 200 mg.

104. The composition of claim 101, wherein the therapeutically effective amount of the RRx-001, or a pharmaceutically acceptable salt thereof, is in a range of about 5 mg and about 50 mg.

105. The composition of claim 101, wherein the therapeutically effective amount of the RRx-001, or a pharmaceutically acceptable salt thereof, is in a range of about 10 mg and about 30 mg.

106. The composition of claim 100, wherein the radiation comprises ionizing radiation.

107. The composition of claim 106, wherein the normal tissue injury is from the ionizing radiation.

108. The composition of claim 100, wherein the normal tissue injury is from chemotherapy.

109. The composition of any one of claims 100-108, wherein the subject has a locally advanced solid tumor.

110. The composition of claim 109, wherein the locally advanced solid tumor is selected from the group consisting of: a gastrointestinal malignancy, rectal cancer, anal cancer, head and neck cancer, gynecological cancer, breast cancer, hepatocellular cancer, esophageal cancer, lung cancer, genitourinary cancer, gastrointestinal tract cancer, genitourinary cancer, hepatocellular carcinoma, glioblastoma, and sarcoma.

111. The composition of any one of claims 100-110, wherein the normal tissue injury comprises a condition selected from the group consisting of: mucositis, dysphagia, dyspepsia, laryngeal inflammation, oral dysesthesia, vomiting, salivary duct inflammation, esophagitis, any gastrointestinal distress, myelosuppression, impotence, infertility, dermatitis, hair loss, and increased creatinine.

112. The composition of any one of claims 100-110, wherein the normal tissue injury comprises secondary cancer development.

113. The composition of any one of claims 100-110, wherein the normal tissue is selectively protected from injury relative to tumor tissue in the subject.

114. The composition of any one of claims 100-113, wherein the subject has a genetic syndrome that predisposes the subject to head and neck cancer, such as Fanconi's anemia, Xeroderma pigmentosum, Ataxia telangiectasia, Li Fraumeni Syndrome, Retinoblastoma, Dyskeratosis congenita, Bloom's Syndrome, or Rothmund-Thompson.

115. The composition of claim 114, wherein the subject has a head and neck cancer attributable to one or more of cigarette smoking, alcohol drinking, infection with human papilloma virus (HPV), or Epstein-Barr virus (EBV).

116. The composition of any one of claims 100-115, wherein the subject is a mammal subject.

117. The composition of claim 116, wherein the mammal subject is a human subject.

118. The composition of claim 116, wherein the mammal subject is an animal subject.

119. The composition of any one of claims 100-118, wherein the composition is administered via oral administration, transdermal administration, administration by inhalation, nasal administration, topical administration, intra-aural administration, intratumoral injection, rectal administration, intravenous administration, intramuscular administration, subcutaneous administration, or intraperitoneal administration.

120. The composition of claim 119, wherein the composition is administered via intravenous administration.

121. The composition of claim 119, wherein the composition is administered via oral administration, and wherein the oral administration comprises a swish and spit administration.

122. The composition of claim 119, wherein the composition is administered via oral administration, and wherein the oral administration comprises a swish and swallow administration.

123. The composition of any one of claims 100-118, wherein the composition is administered via direct intratumoral injection.

124. The composition of any one of claims 100-123, wherein the composition is administered via a single administration.

125. The composition of any one of claims 100-123, wherein the composition is administered via a at least two administrations during a time period.

126. The composition of claim 125, wherein the time period is up to six weeks.

127. The composition of claim 125, wherein administration of the composition is performed at a frequency of between about 1 time per hour and about 1 time per month during the time period.

128. The composition of claim 125, wherein administration of the composition is performed at a frequency of between about 4 times per day and about 1 time per week during the time period.

129. The composition of claim 125, wherein administration of the composition is performed at a frequency of between about 2 times per day and about 3 times per week during the time period.

130. The composition of claim 125, wherein administration of the composition is performed at a frequency of about one time a day during the time period.

131. The composition of claim 125, wherein administration of the composition is performed at a frequency of between about 1 time per day and about 2 times per week during the time period.

132. The composition of any one of claims 100-131, wherein each of the at least one agent is selected from the group consisting of: Dexamethasone, Erbitux (Cetuximab), Avasopasem (GC4419), Pembrolizumab, Nivolumab, Hydrocortisone, Prednisone, Cyclophosphamide, Methotrexate, Paclitaxel, Carboplatin, Etoposide, Gemcitabine, Cisplatin, Oxaliplatin, Chlorambucil, Mechloethamine, Melphalan, Tocilizumab, Brentuximab Vedotin, Doxorubicin, Afatinib, Everolimus, Netupitant, Palonosetron, Imiquimod, and Fluorouracil.

133. The composition of claim 132, wherein an amount of each of the at least one agent is in a range of about 1 mg to about 50 mg.

134. The composition of claim 133, wherein an amount of each of the at least one agent is in the range of about 1 mg to about 15 mg.

135. The composition of any one of claims 100-131, wherein each of the at least one agent comprises a corticosteroid.

136. The composition of claim 135, wherein the corticosteroid comprises dexamethasone.

137. The composition of claim 135, wherein an amount of the agent is in a range of about 0.1 mg to about 50 mg.

138. The composition of any one of claims 100-131, wherein each of the at least one agent is selected from the group consisting of: a PARP inhibitor, a tyrosine kinase inhibitor, a thiol-based chemoradioprotectant agent, an EGFR inhibitor, an HDAC inhibitor, a DNA methyltransferase inhibitor, 5-FU, imatinib, hydroxyurea, taxol, an oncolytic virus, a checkpoint inhibitor, and a topoisomerase inhibitor.

139. The composition of claim 138, wherein each of the at least one agent comprises the thiol-based chemoradioprotectant agent, and wherein the thiol-based chemoradioprotectant agent is selected from the group consisting of: N-acetyl cysteine (NAC), amifostine, sodium thiosulfate (STS), D-methionine, GSH ethyl ester, and GlyNAC (Glycine and N-Acetylcysteine).

140. The composition of claim 138, wherein each of the at least one agent comprises the topoisomerase inhibitor, and wherein the topoisomerase inhibitor is selected from the group consisting of: irinotecan and doxorubicin.

141. The composition of any one of claims 100-131, wherein each of the at least one agent treats oral mucositis.

142. The composition of claim 141, wherein each of the at least one agent that treats oral mucositis is selected from the group consisting of: GM-CSF, palifermin, pilocarpine, gabapentin, keratinocyte growth factor (KGF), coconut oil, MucoLox, cetuximab, a checkpoint inhibitor, transforming growth factor beta (TGF-β), an epidermal growth factor, honey, benzydamine, a corticosteroid, rebamipide, an antimicrobial agent, misoprostol, Amifostine, brilacidin, L-glutamine, low-level laser therapy, laser and light therapy, cryotherapy, vitamin E, pentoxyfyilline, GC4419, clonidine, melatonin, a probiotic, actovegin, aloe vera, allopurinol, azithromycin, Black Mulberry Molasses, Glycerin payayor, Qingre Liyan decoction, erythropoietin mouthwash, EC-18, sucralfate, Avasopasem manganese, and lidocaine, wherein the checkpoint inhibitor is selected from the group consisting of: pembrolizumab and nivolumab, and wherein the antimicrobial agent is selected from the group consisting of: chlorhexidine, polymyxin E, and amphotericin.

143. The composition of any one of claims 100-142, wherein the blood product comprises erythrocyte cells.

144. The composition of claim 143, wherein the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis.

145. The composition of any one of claims 100-142, wherein the blood product is a mixture of packed red blood cells.

146. The composition of any one of claims 100-142, wherein the blood product is whole blood.

147. The composition of claim 146, wherein the whole blood is autologous whole blood or donor-matched allogenic whole blood.

148. The composition of any one of claims 100-147, wherein administration of the composition increases tumor ablation in the subject.

149. The composition of any one of claims 100-148, wherein administration of the composition attenuates secondary cancer development in the subject.

150. The composition of any one of claims 100-149, wherein administration of the composition selectively protects normal tissue from an injury relative to tumor tissue in the subject, and wherein the injury is selected from the group consisting of: mucositis, dysphagia, dyspepsia, laryngeal inflammation, oral dysesthesia, vomiting, salivary duct inflammation, esophagitis, any gastrointestinal distress, myelosuppression, impotence, infertility, dermatitis, hair loss and increased creatinine.

151. The composition of any one of claims 100-150, wherein administration of the composition results in the subject experiencing a decrease in malnutrition.

152. The composition of any one of claims 100-151, wherein administration of the composition results in the subject having a lower incidence of gastrostomy-tube placement.

153. The composition of any one of claims 100-152, wherein administration of the composition results in the subject having a lower incidence of weight loss.

154. The composition of any one of claims 100-153, wherein administration of the composition results in a decrease in other toxicities associated with mucositis.

155. The composition of any one of claims 100-117 and 119-154, wherein the subject comprises the human subject, and wherein administration of the composition increases a tolerated dose of at least one of radiation and chemotherapy in treating the human subject for cancer.

156. The composition of claim 155, wherein the chemotherapy is platinum-based chemotherapy.

157. The composition of claim 156, wherein the platinum-based chemotherapy is selected from the group consisting of: cisplatin, oxaliplatin, carboplatin, nedaplatin, heptaplatin, and lobaplatin.

158. The composition of any one of claims 155-157, wherein increasing the tolerated dose of the at least one of the radiation and the chemotherapy in treating the human subject for cancer comprises increasing a previously poorly tolerated dose of the at least one of the radiation and the chemotherapy.

159. The composition of any one of claims 155-158, wherein an increased tolerated dose or relative dose intensity (RDI) of the at least one of the radiation and the chemotherapy increases subsequent to administration of the composition.

160. The composition of claim 158, wherein the previously poorly tolerated dose of the at least one of the radiation and the chemotherapy becomes tolerable subsequent to administration of the composition.

161. The composition of any one of claims 155-160, wherein the increased tolerated dose of the at least one of the radiation and the chemotherapy comprises administering an increased dose of at least one of cisplatin and radiotherapy each time the at least one of the cisplatin and the radiotherapy are administered.

162. The composition of claim 161, wherein the increased tolerated amount of the cisplatin each time the cisplatin is administered is in a range of about 50 mg to about 150 mg.

163. The composition of any one of claims 161-162, wherein administering the effective amount of the RRx-001, or a pharmaceutically acceptable salt thereof, results in an increased anti-tumor efficacy of the at least one of the cisplatin and the radiotherapy in a treatment for cancer.
